(19) United States
(12) Patent Application Publication
(10) Pub. No.: US 2024/0270809 A1
(43) Pub. Date: Aug. 15, 2024

(54) ORAL GLP RECEPTOR AGONISTS

(71) Applicant: HEPTARES THERAPEUTICS LIMITED, Cambridge, Cambridgeshire (GB)

(72) Inventors: Giles Albert BROWN, Cambridge (GB); Miles Stuart CONGREVE, Cambridge (GB); Conor SCULLY, Cambridge (GB); Rebecca PAUL, Cambridge (GB); Susumu MUTO, Yokohama (JP); Hiroki WADA, Yokohama (JP); Seiji NUKUI, Yokohama (JP)

(73) Assignee: HEPTARES THERAPEUTICS LIMITED, Cambridge, Cambridgeshire (GB)

(21) Appl. No.: 17/909,805

(22) PCT Filed: Mar. 16, 2021
(86) PCT No.: PCT/GB2021/050661

§ 371 Date: Sep. 7, 2022

(30) Foreign Application Priority Data
Mar. 16, 2020 (GB) 2003764.4
Mar. 16, 2020 (GB) 2003766.9

(51) Int. Cl. C07K 14/605; A61K 38/00

(52) U.S. Cl. CPC C07K 14/605; A61K 38/00

(57) ABSTRACT

The disclosures herein relate to novel compounds of formula (1a) or formula (1b): and salts thereof, wherein S, T, W, Z, AA¹, AA², AA³, AA⁴, AA⁵, AA⁶, AA⁷, AA⁸, AA⁹, AA¹⁰, AA¹¹, AA¹², AA¹³, AA¹⁴, AA¹⁵, AA¹⁶, A¹⁷, AA¹⁸, AA¹⁹, AA²⁰, AA²¹, AA²², S^{a}, T^{a}, W^{a}, X^{a}, Y^{a}, Z^{a}, AA^{1a}, AA^{2a}, AA^{3a}, AA^{4a}, AA^{5a}, AA^{6a}, AA^{7a}, AA^{8a}, AA^{9a}, AA^{10a}, AA^{11a}, AA^{12a}, AA¹³ AA^{14a}, AA^{15a}, AA^{16a}, R, R¹ and R² are defined herein, and their use in treating, preventing, ameliorating, controlling or reducing the risk of disorders associated with Glucagon-like peptide (GLP) receptors.

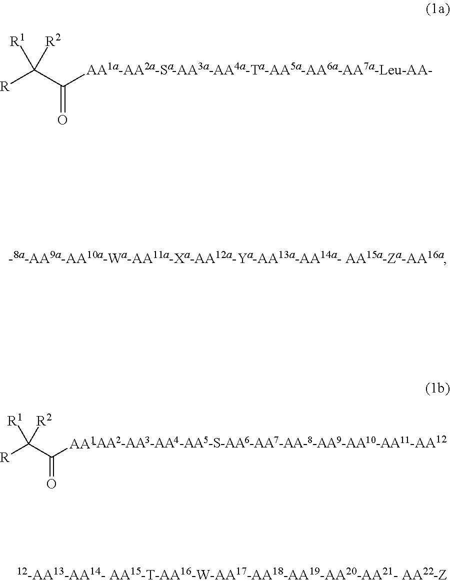

This invention relates to a class of novel orally delivered peptide compounds, their salts, pharmaceutical compositions containing them and their use in therapy of the human body. In particular, the invention is directed to a class of compounds which are agonists of Glucagon-like peptide (GLP) receptors. More particularly, the invention is directed to compounds that are agonists of the Glucagon-like peptide-1 (GLP-1) and Glucagon-like peptide-2 (GLP-2) receptors. More particularly, the invention is directed to compounds that are selective agonists of the Glucagon-like peptide-2 (GLP-2) receptor. The disclosure provides therapeutic methods for treating gastrointestinal diseases through administration of such compounds via the oral route of delivery. The compounds of the invention possess enhanced stability in gastrointestinal relevant fluids. The invention also relates to the manufacture and use of these compounds and compositions in the prevention or treatment of such diseases in which GLP receptors are involved.

### BACKGROUND OF THE INVENTION

Glucagon-like peptide-1 (GLP-1) and Glucagon-like peptide-2 (GLP-2) are highly conserved amino acid peptides that originate from the same precursor protein. These biologically active peptides are encoded by the proglucagon gene which undergoes tissue specific post-translational processing in the pancreas (alpha cells), intestine (L-cells) and the central nervous system (CNS). In the gastrointestinal tract, prohormone convertase 1/3 is responsible for cleaving proglucagon to give rise to a number of biologically active peptides including GLP-1, GLP-2, IP2, oxyntomodulin and glicentin. Both GLP-1 and GLP-2 are secreted in response to nutrient ingestion by intestinal L cells localised in the distal ileum and colon and plasma levels of these gut peptides are reported to be increased after food intake in man.

The actions of GLP-1 and GLP-2 are mediated through the activation of class B G protein coupled receptors, GLP-1R and GLP-2R, which couple to the Gs protein and stimulate CAMP production via activation of adenylate cyclase. GLP-1R is found expressed in the brain, pancreatic islet cells, heart, kidney and myenteric plexus neurones in the gastrointestinal tract. The expression of GLP-2R on the other hand, is more restricted, and the receptor is largely localised to the CNS and the gastrointestinal tract. A number of cell types have been reported to express GLP-2R in the gut including enteric neurons, subepithelial myofibroblasts and enteroendocrine cells, however the exact cellular distribution remains to be defined.

GLP-2 has been reported to be involved in a wide range of physiological functions including gut barrier function, mesenteric blood flow, gastric motility and acid secretion. Exogenous administration of GLP-2 stimulates crypt cell proliferation, enhances intestinal villi length and promotes the growth and repair of the small intestinal mucosa. The potent intestinotrophic activity of GLP-2 has been documented across species including rats, pigs and human. GLP-2 furthermore enhances intestinal absorptive capacity through regulation of intestinal brush border enzymes and solute carriers, highlighting the potential role of this gut hormone in the control of energy homeostasis. Based on the ability to promote potent intestinotrophic effects in the gut, Teduglutide, a GLP-2 analogue has been approved as pharmacological therapy for PN dependent SBS patients and has been shown to reduce PN requirements as well as promote enteral autonomy. In addition to Teduglutide, a number of GLP-2 peptide agonists are in clinical development (e.g. apraglutide, glepaglutide) however all current agents are targeted towards parenteral delivery via subcutaneous injection. GLP peptides that can be given via the oral route of delivery are likely to offer better patient acceptance through convenience of dosing, allow earlier treatment initiation and improve long term compliance. This may particularly be advantageous when considering the development of peptide therapeutics for pediatric patients. However, there are many challenges to the oral delivery of peptides as molecules generally suffer from poor peptide stability (due to extensive proteolytic degradation) as well as low membrane permeability. In the stomach, an orally delivered peptide requires stability in the acidic low pH environment as well as resistance to gastric proteases. In the intestine, the peptides are further subject to degradation from a range of intestinal or pancreatic secreted enzymes as well as brush border membrane bound enzymes. A wide range of biopharmaceutical, formulation and delivery strategies are currently under investigation to overcome some of these barriers. The development of novel potent and stable peptides targeting GLP-2 and GLP-1 receptors suitable for oral delivery remains an attractive strategy and is highly desirable.

GLP-1 is a 31 amino acid peptide which is co-released with GLP-2 in response to luminal nutrients (carbohydrates, fats, proteins) and serves as a gut incretin hormone that works in concert with glucose-dependent insulinotropic polypeptide (GIP). GLP-1 plays a key physiological role in pancreatic islet B-cell function, regulating β-cell proliferation as well as postprandial insulin synthesis/release. Studies have furthermore shown that GLP-1 controls the release of other gut peptides such as somatostatin and glucagon. Following its release, somatostatin acts to suppress GLP-1 and GIP secretion thereby establishing a feedback system in enteroendocrine cells. GLP-1 is a key anorexigenic peptide involved in the regulation of satiety and appetite control, and impacts GI function through effects on gastric emptying and gut motility. Several GLP-1 agents are currently marketed for the treatment of type 2 diabetes mellitus and have been successful in improving glycemic control in diabetic patients. One oral formulation of GLP-1 peptide is currently in clinical development (Semaglutide, Ph III) for the treatment of type 2 diabetes. Once daily formulation of oral semaglutide has shown efficacy superior to active comparators and shows comparable safety and tolerability profile to injectable GLP-1 receptor agonists.

Intestinal failure (IF) refers to a serious and disabling condition whereby the gut is unable to absorb necessary water, electrolytes, macro- and micronutrients for survival. The causes of IF are varied and can result from obstruction, dysmotility, surgical resection, congenital defect or disease associated loss of absorption.

Short bowel syndrome represents the most common cause of intestinal failure and arises from the physical or functional loss of a bowel section, often leading to malnutrition, weight loss, dehydration, diarrhoea, steatorrhoea, fatigue and abdominal pain. Management of SBS requires multidisciplinary care and parenteral nutrition (PN) support to compensate for the extensive fluid loss and to restore nutrient and electrolyte balances. Although critical for survival, long term dependence on parenteral nutrition can negatively impact the patient's quality of life and furthermore increase the risk of life threatening complications such as catheter related sepsis, venous thrombosis and liver damage (e.g. steatosis, cholestasis).

The symptoms and severity of SBS can vary depending on the location and length of the remnant bowel. Intestinal motility is known to be influenced by multiple gut hormones including GLP-1, GLP-2 and PYY which are typically produced by L cells in the ileum and proximal colon. Hormones such as GLP-1 act to provide important feedback mechanisms to control the rate of GI transit for efficient nutrient digestion and absorption. Patients with jejunostomy that have lost the ileal brake have lower fasting GLP-1 and GLP-2 concentrations in plasma and generally suffer rapid gastric emptying and GI transit with high stoma output. Small pilot studies have demonstrated that exenatide or liraglutide (GLP-1 agonists) improve symptoms of diarrhoea in SBS patients and furthermore reduce the requirement for PN.

Adding to the complex clinical picture, evidence also exists for a dysregulated enteroinsular axis in patients with bowel resection that results in impaired insulin response in response to oral glucose administration. In addition, hyperglycemia is a frequent complication of parenteral nutrition in hospitalised patients and can increase the risk of death and infectious complications. The prevalence of hyperglycemia in patients receiving specialised nutritional support is estimated to be up to 30% for those receiving enteral nutrition and 50% in patients on parenteral nutrition. It is recognised that continued poor control of hyperglycemia can lead to a decline in pancreatic beta cell function and can contribute to exacerbating complications such as microvascular disease, cardiovascular events and hypertension. Patients with hyperglycemia during TPN are at greater risk of being admitted to ICU, have longer hospital stays and higher mortality rates compared to those without hyperglycemia.

Based on the known insulinotropic activity of GLP-1 agonists, activation of this mechanism could therefore potentially offer additional benefit in those that develop diminished insulin sensitivity post-surgery and in patients receiving parenteral nutrition. These findings therefore highlight the potential for a combined GLP-2/GLP-1 pharmacological approach in the management of intestinal failure conditions including SBS.

Other intestinal failure conditions where GLP-2/GLP-1 agonists could provide benefit include rare congenital diarrhoeal diseases such as Tufting enteropathy which presents with early onset severe intractable diarrhoea that persists during fasting. Acute treatment of infants with parenteral nutrition, fluid and electrolyte replacement is critically required to prevent dehydration, electrolyte imbalance and impaired growth resulting from severe malnutrition.

Gene encoding the epithelial cell adhesion molecule EpCAM shows association with Tufting enteropathy and to date over 25 EpCAM mutations have been described in the literature. Mutations in the EpCAM gene leads to the loss of cell surface expression, giving rise to the distinctive histological features in the intestinal epithelium, such as focal crowding of enterocytes and formation of ‘tufts’. Mice carrying deletion of exon 4 of the EpCAM gene demonstrate similar morphological defects to Tufting patients with significant morbidity and mortality. EpCAM directly associates with claudin 7, a tight junction molecule and disruptions of this gene leads to poor enterocyte adhesion and impaired gut barrier function, possibly through downregulation of tight junction molecules.

Infants with Tufting enteropathy have low IGF-1 levels and depend on parenteral nutrition to compensate for the diminished capacity to absorb nutrients. Currently there are no pharmacological treatments for this debilitating condition and there is pressing need for agents that can improve intestinal function to promote independence from parenteral feeding. Recent analysis of the long term outcome of Tufting patients has revealed that enteral autonomy can successfully be achieved in most patients if they are effectively managed under specialist care settings. Therapies that promote early weaning are expected to lead to a better long term outcome in these patients and improve the quality of life. Agents acting at GLP-2 and GLP-1 receptors may hold promise in repairing barrier function and aiding recovery of intestinal function in this congenital diarrhoeal disease.

### SUMMARY OF THE INVENTION

The present invention relates to novel compounds with agonist activity at the GLP-2 and GLP-1 receptor, pharmaceutical compositions comprising these, and use of the compounds for the manufacture of medicaments for treatment of diseases. The present disclosure provides therapeutic methods for treating gastrointestinal diseases through administration of such compounds via the oral route of delivery. The compounds of the invention possess enhanced stability in gastrointestinal relevant fluids by having one or more lactam bridges.

Accordingly, in one embodiment the invention provides a compound of the formula (1a):

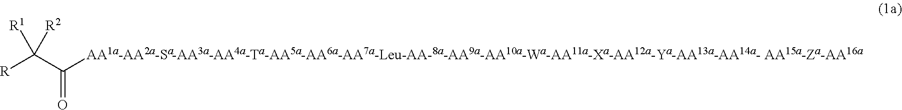

wherein;

R is selected from:

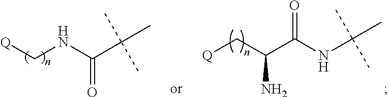

Q is phenyl or a monocyclic heteroaryl ring each of which may be optionally substituted with one or more R^{q} groups;
R^{q} is selected from halogen, hydroxyl, amino or C₁₋₆ alkyl having an alkyl chain optionally containing one or more heteroatoms selected from O, N, or S;
n is 1 to 3;
R¹ and R² are independently selected from hydrogen or a C₁₋₆ alkyl group, or together with the carbon to which they are attached join to form a C₃₋₈ cycloalkyl or a heterocyclyl group;
S^{a} is the sequence -Ser-Phe-;
T^{a} is the sequence -Glu-Nle-;
W^{a} is the sequence -Ala-Ala-;
X^{a} is the sequence -Asp-Phe-Ile-;
Y^{a} is the sequence -Trp-Leu-Ile-;
Z^{a} is absent or is the sequence -Ile-Thr-;
AA^{1a} is —NHCHR³CO—; wherein R³ is selected from —(CH₂)_{y}CONH₂, —(CH₂)_{y}COOH or —(CH₂)_{y}tetrazolyl: where y is 1 or 2;
AA^{2a} is -Gly-, -DAla-, -Lys- optionally joined to AA^{4a} via a lactam bridge or -Glu- optionally joined to AA^{4a} via a lactam bridge;
AA^{3a} is -Ser- or is -Glu- optionally joined to AA^{5a} via a lactam bridge;
AA^{4a} is -Asp- optionally joined to AA^{2a} via a lactam bridge, or -Lys- optionally joined to AA^{2a} or AA^{6a} via a lactam bridge;
AA^{5a} is -DPhe-, -Asp- optionally joined to AA^{8a} via a lactam bridge or -Lys- optionally joined to AA^{3a} via a lactam bridge;
AA^{6a} is -Thr-, -Asp- optionally joined to AA^{4a} or AA^{9a} via a lactam bridge, -Glu- optionally joined to AA^{9a} via a lactam bridge or -Lys- optionally joined to AA^{9a} via a lactam bridge;
AA^{7a} is -Ile- or an a-methyl Leucine residue of formula:

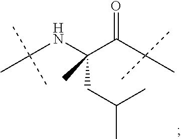

AA^{8a} is -Asp- or is -Lys- optionally joined to AA^{5a} via a lactam bridge;
AA^{9a} is -Leu-, -Lys- optionally joined to AA^{6a} or AA^{11a} via a lactam bridge, -Asp- optionally joined to AA^{6a} or AA^{11a} via a lactam bridge or -Glu- optionally joined to AA^{11a} via a lactam bridge;
AA^{10a} is -Lys- or is -Glu- optionally joined to AA^{11a} via a lactam bridge;
AA^{11a} is -Aib-, -Lys- optionally joined to AA^{9a} or AA^{10a} via a lactam bridge, -Glu- optionally joined to AA^{9a} via a lactam bridge or -Asp- optionally joined to AA^{9a} via a lactam bridge;
AA^{12a} is -Asn-, -Glu- optionally joined to AA^{13a} via a lactam bridge or -Lys- optionally joined to AA^{13a} via a lactam bridge;
AA^{13a} is -Gln-, -Asp- optionally joined to AA^{12a} via a lactam bridge or -Lys- optionally joined to AA^{12a} via a lactam bridge;
AA^{14a} is -Thr- or is -Lys- optionally joined to AA^{16a} via a lactam bridge;
AA^{15a} is -Lys- optionally joined to AA^{16a} via a lactam bridge or -Glu- optionally joined to AA^{16a} via a lactam bridge;
AA^{16a} is absent or is -Asp-, -Phe-, -Lys- optionally joined to AA^{15a} via a lactam bridge or -Glu- optionally joined to AA^{14a} or AA^{15a} via a lactam bridge;
wherein the AA^{15a} or AA^{16a} C-terminus is a carboxyl group or a carboxamide group, or is adjoined to any natural or non-natural amino acid sequence or any other moiety, functional group or groups, and wherein the compound contains one or two lactam bridges;
or a tautomeric or stereochemically isomeric form thereof or a prodrug, salt or zwitterion thereof.

Accordingly, in one embodiment the invention provides a compound of the formula (1b):

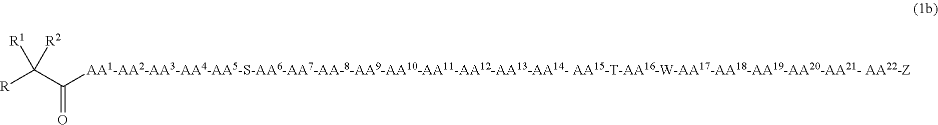

wherein;

R is selected from:

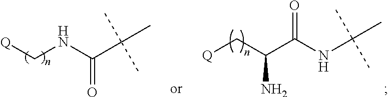

Q is phenyl or a monocyclic heteroaryl ring each of which may be optionally substituted with one or more R^{q} groups;
R^{q} is selected from halogen, hydroxyl, amino or C₁₋₆ alkyl having an alkyl chain optionally containing one or more heteroatoms selected from O, N, or S;
n is 1 to 3;
R¹ and R² are independently selected from hydrogen or a C₁₋₆ alkyl group, or together with the carbon to which they are attached join to form a C₃₋₈ cycloalkyl or a heterocyclyl group;
S is the sequence -Glu-Nle-;
T is the sequence -Phe-Ile-;
W is the sequence -Trp-Leu-Ile-;
Z is absent or is -Pro-;
AA¹ is —NHCHR³CO—; wherein R³ is selected from —(CH₂)_{y}CONH₂, —(CH₂)_{y}COOH or —(CH₂)_{y}tetrazolyl; where y is 1 or 2;
AA² is -Gly-, -DAla-, -Lys- optionally joined to AA⁵ via a lactam bridge or -Glu- optionally joined to AA⁵ via a lactam bridge;
AA³ is -Ser-Phe or -Ser-2-F-α-Me-Phe-;
AA⁴ is -Ser- or -Glu- optionally joined to AA⁶ via a lactam bridge;
AA⁵ is -Asp- optionally joined to AA² via a lactam bridge or -Lys- optionally joined to AA² or AA⁷ via a lactam bridge;
AA⁶ is -D-Phe-, -D-α-Me-Phe- or -Lys- optionally joined to AA¹⁰ via a lactam bridge;
AA⁷ is -Asp- optionally joined to AA⁵ via a lactam bridge, -Glu- optionally joined to AA¹⁰ via a lactam bridge or -Lys- optionally joined to AA¹⁰ via a lactam bridge;
AA⁸ is -Ile or -α-Me-Leu-;
AA⁹ is -Leu-Asp- or -Leu-ACPC-;
AA¹⁰ is -Asp- optionally joined to AA⁷ or AA¹⁴ via a lactam bridge, -Glu- optionally joined to
AA⁷ or AA¹⁴ via a lactam bridge or -Lys- optionally joined to AA⁷ via a lactam bridge;
AA¹¹ is -LysR- where LysR is an N-substituted Lysine residue, -Glu- optionally joined to AA¹⁴ via a lactam bridge or -Lys- optionally joined to AA¹⁵ via a lactam bridge;
AA¹² is -Ala- or -AlB-;
AA¹³ is -Ala- or -AlB-;
AA¹⁴ is -AlB- or is -Lys- optionally joined to AA¹⁰ or AA¹¹ via a lactam bridge;
AA¹⁵ is -Asp- optionally joined to AA¹¹ via a lactam bridge or -Glu- optionally joined to AA¹⁶ via a lactam bridge;
AA¹⁶ is -Asn-, -ACPC-, -Lys- optionally joined to AA¹⁷ via a lactam bridge or -Glu- optionally joined to AA¹⁷ via a lactam bridge;
AA¹⁷ is -Gln-, -ACPC-, -Lys- optionally joined to AA¹⁶ via a lactam bridge or -Glu- optionally joined to AA¹⁶ via a lactam bridge;
AA¹⁸ is -Thr-, -Lys- optionally joined to AA²² via a lactam bridge or -Glu- optionally joined to AA²² via a lactam bridge;
AA¹⁹ is -Pro-, -PIPALA-, -Lys- or -Glu- optionally joined to AA²² via a lactam bridge;
AA²⁰ is absent or is -Ile-, -α-Me-Leu- or -Pro-;
AA²¹ is absent or is -Thr-;
AA²² is absent or is -Lys- optionally joined to AA¹⁸ or AA¹⁹ via a lactam bridge or -Glu- optionally joined to AA¹⁸ via a lactam bridge;
wherein the C-terminus is a carboxyl group or a carboxamide group, or is adjoined to any natural or non-natural amino acid sequence or any other moiety, functional group or groups, and wherein the compound contains three, four or five lactam bridges;
or a tautomeric or stereochemically isomeric form thereof or a prodrug, salt or zwitterion thereof.

The GLP-2/GLP-1 derivatives of this invention can be used in the treatment of various diseases as described below.

In one aspect, the present invention provides a method for promoting growth of small bowel tissue in a patient in need thereof, comprising the step of delivering to the patient an intestinotrophic amount of a GLP-2/GLP-1 analogue of the present invention.

In a further aspect the present invention relates to a method for the preparation of a medicament for the treatment of gastrointestinal diseases that include intestinal failure or other conditions leading to nutrient malabsorption and intestinal insufficiency. Examples of such diseases may include small bowel syndrome, diarrhoeal diseases, inflammatory bowel syndrome, Crohn's disease, Ulcerative colitis, pouchitis, radiation induced bowel damage, Celiac disease (gluten sensitive enteropathy), NSAID-induced gastrointestinal damage, cancer treatment induced tissue damage (e.g. chemotherapy induced enteritis), Parkinson's disease, parenteral nutrition induced mucosal atrophy, intestinal failure in preborn infants, necrotizing enterocolitis, neonatal feeding intolerance, congenital diarrhoeal diseases, congenital or acquired digestion and absorption disorders, tissue damage induced by vascular obstruction, trauma or ischemia.

A further aspect of the invention is a method for treating the symptoms of, or treating rare congenital diarrheal diseases in a patient in need thereof, by delivering a GLP-2/GLP-1 analogue of the present invention in a therapeutically effective amount. Persistent uncontrolled diarrhoea can cause severe dehydration, electrolyte imbalance, malnutrition and failure to thrive which, if left untreated, could lead to life threatening condition including death.

In a further aspect, the present invention provides the use of a compound as outlined above for the preparation of a medicament for the treatment of Tufting enteropathy, a rare congenital diarrhoeal disease characterised by early onset severe and intractable diarrhoea that often leads to intestinal failure.

A further aspect of the invention is a method for treating metabolic diseases and syndromes in a patient in need thereof, by delivering a GLP-2/GLP-1 analogue of the present invention in a therapeutically effective amount, In one embodiment metabolic disease and syndromes include obesity, type 2 diabetes, non-alcoholic fatty liver disease (NAFLD), nonalcoholic steatohepatitis (NASH), insulin resistance, hyperglycemia, insulin resistance, glucose intolerance. It is envisaged that treatment with a GLP-2/GLP-1 analogue may restore glycemic control and insulin sensitivity. This could be beneficial for the management of hyperglycemia during enteral and parenteral nutrition therapy in patients with intestinal failure, insufficiency or malabsorption disorders.

### DETAILED DESCRIPTION OF THE INVENTION

This invention relates to novel compounds. The invention also relates to the use of novel compounds as agonists of GLP receptors. The invention further relates to the use of novel compounds in the manufacture of medicaments for use as GLP receptor agonists or for the treatment of gastrointestinal and metabolic disorders. The invention further relates to compounds, compositions and medicaments which are selective GLP-2 receptor agonists. The present disclosure provides therapeutic methods for treating gastrointestinal diseases through administration of such compounds via the oral route of delivery. The compounds of the invention possess enhanced stability in gastrointestinal relevant fluids.

Compounds of the invention contain one of more lactam bridges.

Accordingly, in one embodiment the invention provides a compound of the formula (1a):

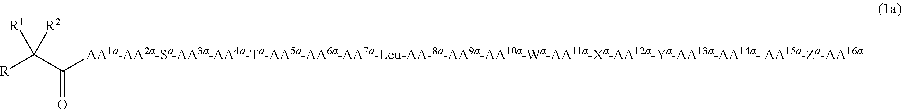

wherein;

R is selected from:

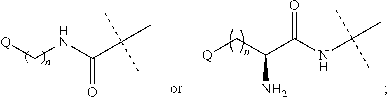

Q is phenyl or a monocyclic heteroaryl ring each of which may be optionally substituted with one or more R^{q} groups;
R^{q} is selected from halogen, hydroxyl, amino or C₁₋₆ alkyl having an alkyl chain optionally containing one or more heteroatoms selected from O, N, or S;
n is 1 to 3;
R¹ and R² are independently selected from hydrogen or a C₁₋₆ alkyl group, or together with the carbon to which they are attached join to form a C₃₋₈ cycloalkyl or a heterocyclyl group;
S^{a} is the sequence -Ser-Phe-;
T^{a} is the sequence -Glu-Nle-;
W^{a} is the sequence -Ala-Ala-;
X^{a} is the sequence -Asp-Phe-Ile-;
Y^{a} is the sequence -Trp-Leu-Ile-;
Z^{a} is absent or is the sequence -Ile-Thr-;
AA^{1a} is —NHCHR³CO—; wherein R³ is selected from —(CH₂)_{y}CONH₂, —(CH₂)_{y}COOH or —(CH₂)_{y}tetrazolyl; where y is 1 or 2;
AA^{2a} is -Gly-, -DAla-, -Lys- optionally joined to AA^{4a} via a lactam bridge or -Glu- optionally joined to AA^{4a} via a lactam bridge;
AA^{3a} is -Ser- or is -Glu- optionally joined to AA^{5a} via a lactam bridge;
AA^{4a} is -Asp- optionally joined to AA^{2a} via a lactam bridge, or -Lys- optionally joined to AA^{2a} or AA^{6a} via a lactam bridge;
AA^{5a} is -DPhe-, -Asp- optionally joined to AA^{8a} via a lactam bridge or -Lys- optionally joined to AA^{3a} via a lactam bridge;
AA^{6a} is -Thr-, -Asp- optionally joined to AA^{4a} or AA^{9a} via a lactam bridge, -Glu- optionally joined to AA^{9a} via a lactam bridge or -Lys- optionally joined to AA^{9a} via a lactam bridge;
AA^{7a} is -Ile- or an α-methyl Leucine residue of formula:

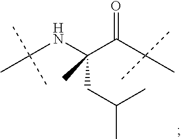

AA^{8a} is -Asp- or is -Lys- optionally joined to AA⁵⁸ via a lactam bridge;
AA^{9a} is -Leu-, -Lys- optionally joined to AA^{6a} or AA^{11a} via a lactam bridge, -Asp- optionally joined to AA^{6a} or AA^{11a} via a lactam bridge or -Glu- optionally joined to AA^{11a} via a lactam bridge;
AA^{10a} is -Lys- or is -Glu- optionally joined to AA^{11a} via a lactam bridge;
AA^{11a} is -Aib-, -Lys- optionally joined to AA^{9a} or AA^{10a} via a lactam bridge, -Glu- optionally joined to AA^{9a} via a lactam bridge or -Asp- optionally joined to AA^{9a} via a lactam bridge;
AA^{12a} is -Asn-, -Glu- optionally joined to AA^{13a} via a lactam bridge or -Lys- optionally joined to AA^{13a} via a lactam bridge;
AA^{13a} is -Gln-, -Asp- optionally joined to AA^{12a} via a lactam bridge or -Lys- optionally joined to AA^{12a} via a lactam bridge;
AA^{14a} is -Thr- or is -Lys- optionally joined to AA^{16a} via a lactam bridge;
AA^{15a} is -Lys- optionally joined to AA^{16a} via a lactam bridge or -Glu- optionally joined to AA^{16a} via a lactam bridge;
AA^{16a} is absent or is -Asp-, -Phe-, -Lys- optionally joined to AA^{15a} via a lactam bridge or -Glu- optionally joined to AA^{14a} or AA^{15a} via a lactam bridge;
wherein the AA^{15a} or AA^{16a} C-terminus is a carboxyl group or a carboxamide group, or is adjoined to any natural or non-natural amino acid sequence or any other moiety, functional group or groups, and wherein the compound contains one or two lactam bridges;
or a tautomeric or stereochemically isomeric form thereof or a prodrug, salt or zwitterion thereof.

Accordingly, in one embodiment the invention provides a compound of the formula (1b):

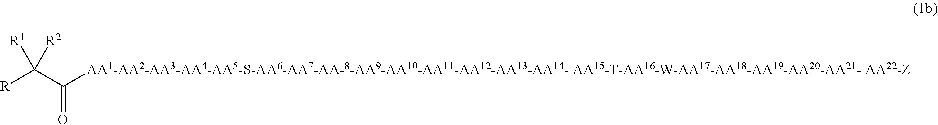

wherein;

R is selected from:

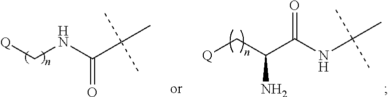

Q is phenyl or a monocyclic heteroaryl ring each of which may be optionally substituted with one or more R^{q} groups;
R^{q} is selected from halogen, hydroxyl, amino or C₁₋₆ alkyl having an alkyl chain optionally containing one or more heteroatoms selected from O, N, or S;
n is 1 to 3;
R¹ and R² are independently selected from hydrogen or a C₁₋₆ alkyl group, or together with the carbon to which they are attached join to form a C₃₋₈ cycloalkyl or a heterocyclyl group;
S is the sequence -Glu-Nle-;
T is the sequence -Phe-Ile-;
W is the sequence -Trp-Leu-Ile-;
Z is absent or is -Pro-;
AA¹ is —NHCHR³CO—; wherein R³ is selected from —(CH₂)_{y}CONH₂, —(CH₂)_{y}COOH or —(CH₂)_{y}tetrazolyl; where y is 1 or 2;
AA² is -Gly-, -DAla-, -Lys- optionally joined to AA⁵ via a lactam bridge or -Glu- optionally joined to AA⁵ via a lactam bridge;
AA³ is -Ser-Phe or -Ser-2-F-α-Me-Phe-;
AA⁴ is -Ser- or -Glu- optionally joined to AA⁶ via a lactam bridge;
AA⁵ is -Asp- optionally joined to AA² via a lactam bridge or -Lys- optionally joined to AA² or AA⁷ via a lactam bridge;
AA⁶ is -D-Phe-, D-α-Me-Phe or -Lys- optionally joined to AA¹⁰ via a lactam bridge;
AA⁷ is -Asp- optionally joined to AA⁵ via a lactam bridge, -Glu- optionally joined to AA¹⁰ via a lactam bridge or -Lys- optionally joined to AA¹⁰ via a lactam bridge;
AA⁸ is -Ile or -α-Me-Leu-;
AA⁹ is -Leu-Asp- or -Leu-ACPC-;
AA¹⁰ is -Asp- optionally joined to AA⁷ or AA¹⁴ via a lactam bridge, -Glu- optionally joined to AA⁷ or AA¹⁴ via a lactam bridge or -Lys- optionally joined to AA⁷ via a lactam bridge;
AA¹¹ is -LysR- where LysR is an N-substituted Lysine residue, -Glu- optionally joined to AA¹⁴ via a lactam bridge or -Lys- optionally joined to AA¹⁵ via a lactam bridge;
AA¹² is -Ala- or -AlB-;
AA¹³ is -Ala- or -AlB-;
AA¹⁴ is -AlB- or is -Lys- optionally joined to AA¹⁰ or AA¹¹ via a lactam bridge;
AA¹⁵ is -Asp- optionally joined to AA¹¹ via a lactam bridge or -Glu- optionally joined to AA¹⁶ via a lactam bridge;
AA¹⁶ is -Asn-, -ACPC-, -Lys- optionally joined to AA¹⁷ via a lactam bridge or -Glu- optionally joined to AA¹⁷ via a lactam bridge;
AA¹⁷ is -Gln-, -ACPC-, -Lys- optionally joined to AA¹⁶ via a lactam bridge or -Glu- optionally joined to AA¹⁶ via a lactam bridge;
AA¹⁸ is -Thr-, -Lys- optionally joined to AA²² via a lactam bridge or -Glu- optionally joined to AA²² via a lactam bridge;
AA¹⁹ is -Pro-, -PIPALA-, -Lys- or -Glu- optionally joined to AA²² via a lactam bridge;
AA²⁰ is absent or is -Ile-, -α-Me-Leu- or -Pro-;
AA²¹ is absent or is -Thr-;
AA²² is absent or is -Lys- optionally joined to AA¹⁸ or AA¹⁹ via a lactam bridge or -Glu- optionally joined to AA¹⁸ via a lactam bridge;
wherein the C-terminus is a carboxyl group or a carboxamide group, or is adjoined to any natural or non-natural amino acid sequence or any other moiety, functional group or groups, and wherein the compound contains three, four or five lactam bridges;
or a tautomeric or stereochemically isomeric form thereof or a prodrug, salt or zwitterion thereof.

All compounds described herein may contain at least one lactam bridges to internally cyclise the peptide sequence All compounds described herein may contain one, two, three, four or five lactam bridges to internally cyclise the peptide sequence. The lactam bridge can be between the side chain amino group of a lysine moiety and the side chain carboxylate group of aspartic acid or glutamic acid. Specifically the lysine can be at positions AA^{2a}, AA^{4a}, AA^{5a}, AA^{6a}, AA^{8a}, AA^{9a}, AA^{11a}, AA^{12a}, AA^{13a}, AA^{14a}, AA^{15a} or AA^{16a}, The aspartic acid or glutamic acid can be at positions AA^{2a}, AA^{3a}, AA^{4a}, AA^{5a}, AA^{6a}, AA^{9a}, AA^{10a}, AA^{11a}, AA^{12a}, AA^{13a}, AA^{15a} or AA^{16a}.

The compounds must include one or two lactam bridges between the amino acid pairs shown below:

AA^{2a}-AA^{4a}; AA^{3a}-AA^{5a}; AA^{4a}-AA^{6a}; AA^{5a}-AA^{8a}; AA^{6a}-AA^{9a};
AA^{9a}-AA^{11a}, AA^{10a}-AA^{11a}, AA^{12a}-AA^{13a}; AA^{14a}-AA^{16a},
AA^{15a}-AA^{16a}.

With the proviso that the compounds at least one lactam bridges, the amino acids can be independently selected from each of the groups shown below.

AA^{1a} can be —NHCHR³CO—; wherein —(CH₂)_{y}tis —(CH₂)_{y}tetrazolyl, where y is 1.

AA^{1a} can be —NHCHR³CO—; wherein R³ is —(CH₂)_{y}tetrazolyl, where y is 2.

AA^{1a} can be —NHCHR³CO—; wherein R³ is —(CH₂)_{y}COOH, where y is 1.

AA^{1a} can be —NHCHR³CO—; wherein R³ is —(CH₂)_{y}COOH, where y is 2.

R³ can be —CH₂COOH.

AA^{1a} can be

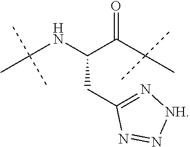

AA^{1a} can be -Asp-. AA^{1a} can be an aspartic acid residue. AA^{1a} can be

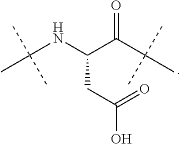

Q can be an imidazole ring. Q can be:

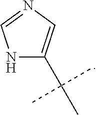

n can be 1. n can be 2. n can be 3.

R¹ and R² may be independently selected from hydrogen or a C₁₋₆ alkyl group. R¹ can be hydrogen or a C₁₋₆ alkyl group. R² can be hydrogen or a C₁₋₆ alkyl group. R¹ and R² can both be methyl. R¹ can be methyl. R² can be methyl.

R³ can be —CH₂tetrazolyl. R³ can be —CH₂COOH.

AA^{2a} can be -Gly-. AA^{2a} can be -DAla-. AA^{2a} can be -Lys-. The lysine can optionally be joined to AA^{4a} via a lactam bridge. AA^{2a} can be -Glu-. The glutamic acid can optionally be joined to AA^{4a} via a lactam bridge.

AA^{3a} can be -Ser-. AA^{3a} can be -Glu-. The glutamic acid can optionally be joined to AA^{5a} via a lactam bridge.

AA^{4a} can be -Asp-. The aspartic acid can optionally be joined to AA^{2a} via a lactam bridge. AA^{4a} can be -Lys-. The lysine can optionally be joined to AA^{2a} or AA^{6a} via a lactam bridge.

AA^{5a} can be -DPhe-. AA^{5a} can be -Asp-. The aspartic acid can optionally be joined to AA^{8a} via a lactam bridge. AA^{5a} can be -Lys- the lysine can optionally be joined to AA^{3a} via a lactam bridge.

AA^{6a} can be -Thr-. AA^{6a} can be -Asp-. The aspartic acid can optionally be joined to AA^{4a} via a lactam bridge. The aspartic acid can optionally be joined to AA^{9a} via a lactam bridge. AA^{6a} can be -Glu-. The glutamic acid can optionally be joined to AA^{9a} via a lactam bridge. AA^{6a} can be -Lys-. The lysine can optionally be joined to AA^{9a} via a lactam bridge.

AA^{7a} can be -Ile-. AA^{7a} can be an α-methyl Leucine residue of formula:

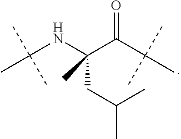

AA^{8a} can be -Asp-. AA^{8a} can be -Lys-. The lysine can be optionally joined to AA^{5a} via a lactam bridge.

AA^{9a} can be -Leu-. AA^{9a} can be -Lys-. The lysine can be optionally joined to AA^{6a} via a lactam bridge. The lysine can be optionally joined to AA^{11a} via a lactam bridge. AA^{9a} can be -Asp-. The aspartic acid can optionally be joined to AA^{6a} via a lactam bridge. The aspartic acid can optionally be joined to AA^{11a} via a lactam bridge. AA^{9a} can be -Glu-. The glutamic acid can optionally be joined to AA^{11a} via a lactam bridge.

AA^{10a} can be -Lys-. AA^{10a} can be -Glu-. The glutamic acid can be optionally joined to AA^{11a} via a lactam bridge.

AA^{11a} can be -Aib-. AA^{11a} can be -Lys-. The lysine can be optionally joined to AA^{3a} via a lactam bridge. The lysine can be optionally joined to AA^{10a} via a lactam bridge. AA^{11a} can be -Glu-. The glutamic acid can be optionally joined to AA^{9a} via a lactam bridge. AA^{11a} can be -Asp-. The aspartic acid can be optionally joined to AA^{9a} via a lactam bridge.

AA^{12a} can be -Asn-. AA^{12a} can be -Glu-. The glutamic acid can be optionally joined to AA^{13a} via a lactam bridge. AA^{12a} can be -Lys-. The lysine can be optionally joined to AA^{13a} via a lactam bridge.

AA^{13a} can be -Gln-. AA^{13a} can be -Asp-. The aspartic acid can be optionally joined to AA^{12a} via a lactam bridge. AA^{13a} can be -Lys-. The lysine can be optionally joined to AA^{12a} via a lactam bridge.

AA^{14a} can be -Thr-. AA^{14a} can be -Lys-. The lysine can be optionally joined to AA^{16a} via a lactam bridge.

AA^{15a} can be -Lys-. The lysine can be optionally joined to AA^{16a} via a lactam bridge. AA^{15a} can be -Glu-. The glutamic acid can be optionally joined to AA^{16a} via a lactam bridge.

AA^{16a} can be absent. Where AA^{16a} is present AA^{16a} can be -Asp-, Where AA^{16a} is present AA^{16a} can be -Phe-. Where AA^{16a} is present AA^{16a} can be -Lys-. The lysine can be optionally joined to AA^{15a} via a lactam bridge. Where AA^{16a} is present AA^{16a} can be -Glu-. The glutamic acid can be optionally joined to AA^{14a} or AA^{15a} via a lactam bridge. The glutamic acid can be optionally joined to AA^{14a} or AA^{15a} via a lactam bridge.

Z^{a} can be absent. Z^{a} can be the sequence -Ile-Thr-.

The AA^{15a} or AA^{16a} C-terminus can be a carboxyl group. The AA^{15a} or AA^{16a} C-terminus can be a carboxamide group. The AA^{15a} or AA^{16a} C-terminus can be adjoined to any natural or non-natural amino acid sequence or any other moiety, functional group or groups.

The compound can be selected from any one of Examples 82 to 117 shown in Table 1a.

Compounds described herein may contain three, four or five lactam bridges to internally cyclise the peptide sequence. The lactam bridge can be between the side chain amino group of a lysine moiety and the side chain carboxylate group of aspartic acid or glutamic acid. Specifically the lysine can be at positions AA², AA⁵, AA⁶, AA⁷, AA¹⁰, AA¹¹, AA¹⁴, AA¹⁶, AA¹⁷, AA¹⁸ or AA²². The aspartic acid or glutamic acid can be at positions AA², AA⁵, AA⁷, AA¹⁰, AA¹¹, AA¹⁵, AA¹⁶, AA¹⁷, AA¹⁸, AA¹⁹ or AA²².

The compounds may include three, four or five lactam bridges between the amino acid pairs shown below:

AA²-AA⁵; AA⁴-AA⁶; AA⁵-AA⁷; AA⁶-AA¹⁰; AA⁷-AA¹⁰;
AA¹⁰-AA¹⁴; AA¹¹-AA¹⁴; AA¹¹-AA¹⁵; AA¹⁶-AA¹⁷;
AA¹⁸-AA²² or AA¹⁹-AA²²;

Exemplary compounds having three bridges include compounds having a first bridge from position AA⁵-AA⁷; a second bridge from position AA¹⁰-AA¹⁴ and a third bridge from position AA¹⁹-AA²².

Exemplary compounds having three bridges include compounds having a first bridge from position AA²-AA⁵; a second bridge from position AA⁷-AA¹⁰ and a third bridge from position AA¹⁶-AA¹⁷.

Exemplary compounds having three bridges include compounds having a first bridge from position AA⁵-AA⁷; a second bridge from position AA¹⁰-AA¹⁴ and a third bridge from position AA¹⁸-AA²².

Exemplary compounds having three bridges include compounds having a first bridge from position AA²-AA⁵; a second bridge from position AA¹⁰-AA¹⁴ and a third bridge from position AA¹⁸-AA²².

Exemplary compounds having four bridges include compounds having a first bridge from position AA²-AA⁵; a second bridge from position AA⁷-AA¹⁰; a third bridge from position AA¹⁶-AA¹⁷ and a fourth bridge from position AA¹⁸-AA²².

Exemplary compounds having four bridges include compounds having a first bridge from position AA⁵-AA⁷; a second bridge from position AA¹⁰-AA¹⁴; a third bridge from position AA¹⁶-AA¹⁷ and a fourth bridge from position AA¹⁹-AA²².

Exemplary compounds having four bridges include compounds having a first bridge from position AA²-AA⁵; a second bridge from position AA⁷-AA¹⁰; a third bridge from position AA¹⁶-AA¹⁷ and a fourth bridge from position AA¹⁹-AA²².

Exemplary compounds having four bridges include compounds having a first bridge from position AA⁵-AA⁷; a second bridge from position AA¹⁰-AA¹⁴; a third bridge from position AA¹⁶-AA¹⁷ and a fourth bridge from position AA¹⁸-AA²².

Exemplary compounds having four bridges include compounds having a first bridge from position AA⁷-AA¹⁰; a second bridge from position AA¹¹-AA¹⁴; a third bridge from position AA¹⁶-AA¹⁷ and a fourth bridge from position AA¹⁸-AA²².

Exemplary compounds having four bridges include compounds having a first bridge from position AA⁷-AA¹⁰; a second bridge from position AA¹¹-AA¹⁵; a third bridge from position AA¹⁶-AA¹⁷ and a fourth bridge from position AA¹⁸-AA²².

Exemplary compounds having four bridges include compounds having a first bridge from position AA²-AA⁵; a second bridge from position AA¹⁰-AA¹⁴; a third bridge from position AA¹⁶-AA¹⁷ and a fourth bridge from position AA¹⁹-AA²².

Exemplary compounds having five bridges include compounds having a first bridge from either position AA²-AA⁵ or position AA⁴-AA⁶; a second bridge from position AA⁷-AA¹⁰ a third bridge from either position AA¹¹-AA¹⁴ or AA¹¹-AA¹⁵; a fourth bridge from position AA¹⁶-AA¹⁷ and a fifth bridge from position AA¹⁸-AA²².

Exemplary compounds having five bridges include compounds having a first bridge from position AA²-AA⁵; a second bridge from position AA⁷-AA¹⁰ a third bridge from position AA¹¹ AA¹⁴, a fourth bridge from position AA¹⁶-AA¹⁷ and a fifth bridge from position AA¹⁸-AA²².

Exemplary compounds having five bridges include compounds having a first bridge from position AA²-AA⁵; a second bridge from position AA⁷-AA¹⁰ a third bridge from position AA¹¹-AA¹⁵, a fourth bridge from position AA¹⁶-AA¹⁷ and a fifth bridge from position AA¹⁸-AA²².

Exemplary compounds having five bridges include compounds having a first bridge from position AA⁴-AA⁶; a second bridge from position AA⁷-AA¹⁰ a third bridge from position AA¹¹-AA¹⁴, a fourth bridge from position AA¹⁶-AA¹⁷ and a fifth bridge from position AA¹⁸-AA²².

Exemplary compounds having five bridges include compounds having a first bridge from position AA⁴-AA⁶; a second bridge from position AA⁷-AA¹⁰ a third bridge from position AA¹¹-AA¹⁵, a fourth bridge from position AA¹⁶-AA¹⁷ and a fifth bridge from position AA¹⁸-AA²².

With the proviso that the compounds contain three, four or five lactam bridges, the amino acids can be independently selected from each of the groups shown below.

AA¹ can be —NHCHR³CO—; wherein R³ is —(CH₂)_{y}tetrazolyl, where y is 1.

AA¹ can be —NHCHR³CO—; wherein R³ is —(CH₂)_{y}tetrazolyl, where y is 2.

AA¹ can be —NHCHR³CO—; wherein R³ is —(CH₂)_{y}COOH, where y is 1.

AA¹ can be —NHCHR³CO—; wherein R³ is —(CH₂)_{y}COOH, where y is 2.

R³ can be —CH₂COOH.

AA¹ can be

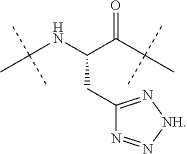

AA¹ can be -Asp-. AA¹ can be an aspartic acid residue. AA¹ can be

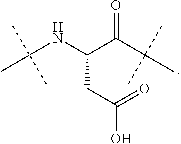

Q can be an imidazole ring. Q can be:

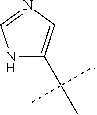

n can be 1. n can be 2. n can be 3.

R¹ and R² may be independently selected from hydrogen or a C₁₋₆ alkyl group. R¹ can be hydrogen or a C₁₋₆ alkyl group. R² can be hydrogen or a C₁₋₆ alkyl group. R¹ and R² can both be methyl. R¹ can be methyl. R² can be methyl.

R³ can be —CH₂tetrazolyl. R³ can be —CH₂COOH.

AA² can be -Gly-. AA² can be -DAla-, AA² can be -Lys-. The lysine can be optionally joined to AA⁵ via a lactam bridge. AA² can be -Glu-. The glutamic acid can be optionally joined to AA⁵ via a lactam bridge.

AA³ can be -Ser-Phe-. AA³ can be -Ser-2-Fluoro-α-Me-Phe-.

AA⁴ can be -Ser-. AA⁴ can be -Glu-. The glutamic acid can be optionally joined to AA⁶ via a lactam bridge.

AA⁵ can be -Asp-. The aspartic acid can be optionally joined to AA² via a lactam bridge. AA⁵ can be -Lys-. The lysine can be optionally joined to AA² or AA⁷ via a lactam bridge.

AA⁶ can be -D-Phe-. AA⁶ can be -D-α-Me-Phe. AA⁶ can be -Lys-. The lysine can be optionally joined to AA¹⁰ via a lactam bridge.

AA⁷ can be -Asp-. The aspartic acid can be optionally joined to AA⁵ via a lactam bridge. AA⁷ can be -Glu-. The glutamic acid can be optionally joined to AA¹⁰ via a lactam bridge. AA⁷ can be or -Lys-. The lysine can be optionally joined to AA¹⁰ via a lactam bridge.

AA⁸ can be -Ile-. AA⁸ can be -α-Me-Leu-.

AA⁹ can be -Leu-Asp-. AA⁹ can be -Leu-ACPC-.

AA¹⁰ can be -Asp-. The aspartic acid can be optionally joined to AA⁷ via a lactam bridge. The aspartic acid can be optionally joined to AA¹⁴ via a lactam bridge. AA¹¹ can be -Glu-. The glutamic acid can be optionally joined to AA¹⁴ via a lactam bridge. The glutamic acid can be optionally joined to AA⁷ via a lactam bridge. AA¹⁰ can be -Lys-. The lysine can be optionally joined to AA⁷ via a lactam bridge;

AA¹¹ can be -LysR- where LysR is an N-substituted Lysine residue. AA¹¹ can be -Glu-. The glutamic acid can be optionally joined to AA¹⁴ via a lactam bridge. AA¹¹ can be -Lys-. The lysine can be optionally joined to AA¹⁵ via a lactam bridge;

LysR can be an N-substituted Lysine residue, wherein the N-substituent is selected from: —CO(CH₂)_{q}CH₃; —CO(CH₂)_{q}CO₂H; —CO(CH₂)_{q}CHCH₂; —COO(CH₂)_{q}CH₃; —COO(CH₂)_{q}CO₂H and —COO(CH₂)_{q}CHCH₂; where q is 1 to 22.

LysR can be an N-substituted Lysine residue, wherein the N-substituent is —COO(CH₂)_{q}CHCH₂; where q is 1 to 22. LysR can be an N-substituted Lysine residue, wherein the N-substituent is —COO(CH₂)_{q}CHCH₂; where q is 1. LysR can be an N-substituted Lysine residue, wherein the N-substituent is —COOCH₂CHCH₂.

Lys R can be

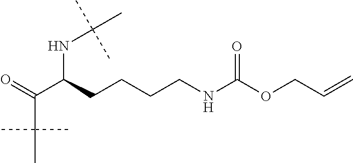

LysR can be an N-substituted Lysine residue, wherein the N-substituent is a group -L-G;
wherein L is selected from the group consisting of:

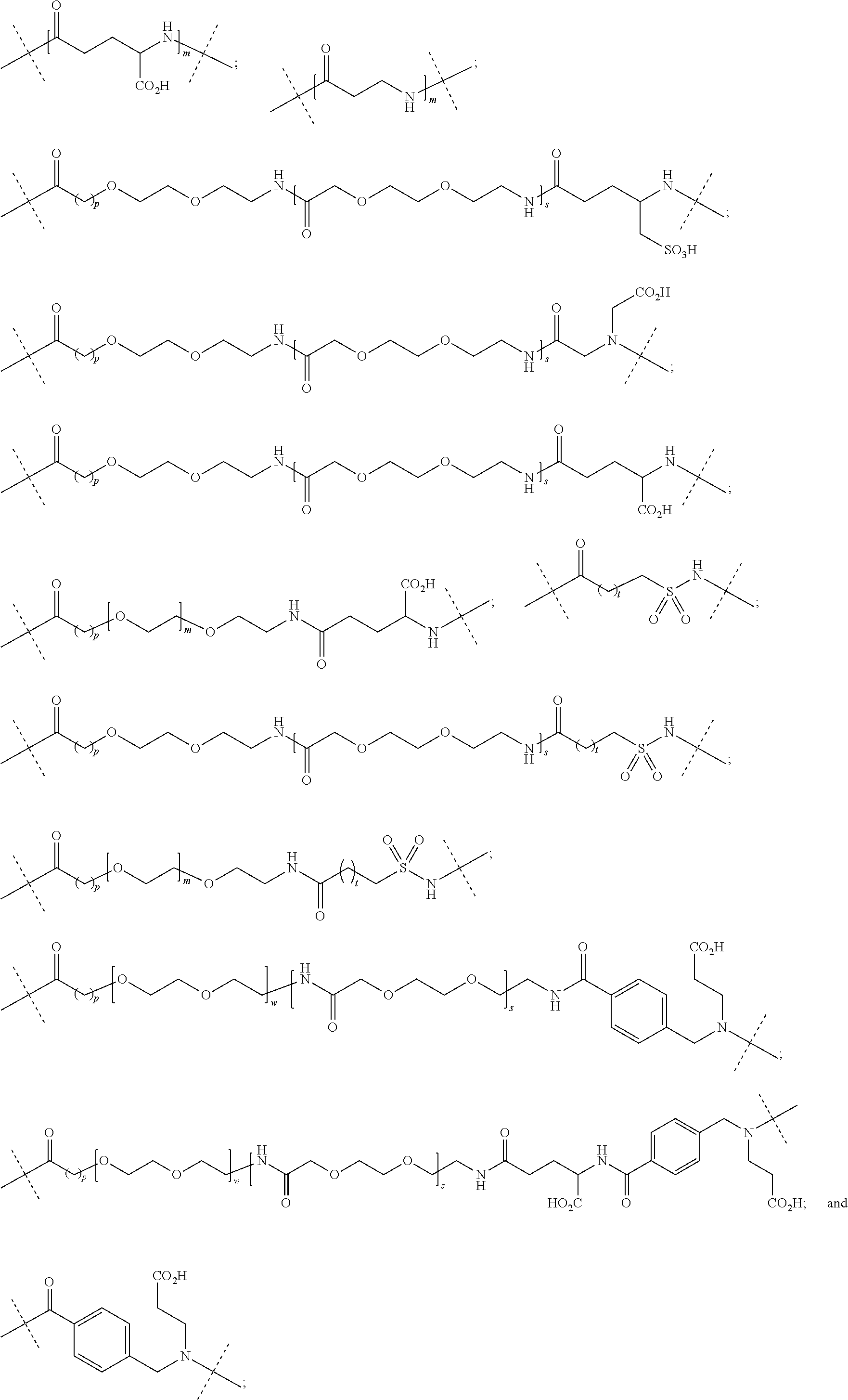

and G is selected from the group consisting of:

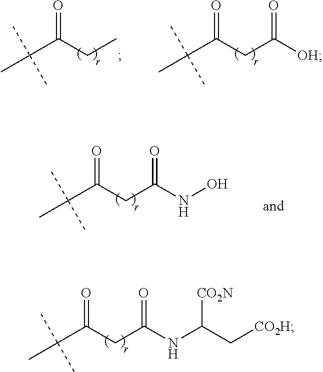

where m is 1 to 23;
p is 1 to 3;
r is 1 to 20;
s is 0 to 3;
t is 0 to 4;
and w is 0 to 4

LysR can be

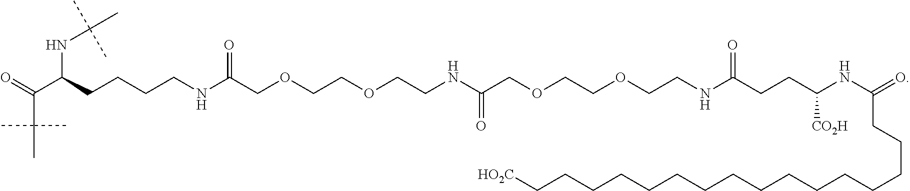

LysR can be

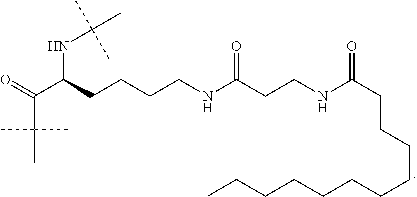

AA¹² can be -Ala-. AA¹² can be -AlB-.

AA¹³ can be -Ala-. AA¹³ can be -AlB-.

AA¹⁴ can be -AlB-. AA¹⁴ can be -Lys-. The lysine can be optionally joined to AA¹⁰ via a lactam bridge. The lysine can be optionally joined to AA¹¹ via a lactam bridge.

AA¹⁵ can be -Asp-. The aspartic acid can be optionally joined to AA¹¹ via a lactam bridge.

AA¹⁵ can be -Glu-. The glutamic acid can be optionally joined to AA¹⁶ via a lactam bridge.

AA¹⁶ can be -Asn-. AA¹⁶ can be -ACPC-. AA¹⁶ can be -Lys-. The lysine can be optionally joined to AA¹⁷ via a lactam bridge. AA¹⁶ can be -Glu-. The glutamic acid can be optionally joined to AA¹⁷ via a lactam bridge.

AA¹⁷ can be -Gln-. AA¹⁷ can be -ACPC-. AA¹⁷ can be -Lys-. The lysine can be optionally joined to AA¹⁶ via a lactam bridge. AA¹⁷ can be -Glu-. The glutamic acid can be optionally joined to AA¹⁶ via a lactam bridge.

AA¹⁸ can be -Thr-. AA¹⁸ can be -Lys-. The lysine can be optionally joined to AA²² via a lactam bridge. AA¹⁸ can be -Glu-. The glutamic acid can be optionally joined to AA²² via a lactam bridge.

AA¹⁹ can be -Pro-. AA¹⁹ can be -PIPALA-. AA¹⁹ can be -Lys-. AA¹⁹ can be or -Glu-. The glutamic acid can be optionally joined to AA²² via a lactam bridge.

AA²⁰ can be absent such that AA¹⁹ is the C-terminus. AA²⁰ can be -Ile-. AA²⁰ can be -α-Me-Leu-. AA²⁰ can be -Pro-.

AA²¹ can be absent such that AA¹⁹ or AA²⁰ is the C-terminus. AA²¹ can be -Thr-.

AA²² can be absent such that AA¹⁹, AA²⁰ or AA²¹ is the C-terminus. AA²² can be -Lys-. The lysine can be optionally joined to AA¹⁸ via a lactam bridge. The lysine can be optionally joined to AA¹⁹ via a lactam bridge. AA²² can be -Glu-. The glutamic acid can be optionally joined to AA¹⁸ via a lactam bridge.

The C terminus can be a carboxyl group. The C terminus can be a carboxamide group. The C terminus can be adjoined to any natural or non-natural amino acid sequence or any other moiety, functional group or groups.

The compound can be selected from any one of Examples 1 to 81 shown in Table 1.

The compound can be selected from any one of Examples 1 to 117 shown in Table 1 and Table 1a.

Specific examples of compounds include compounds having GLP receptor agonist activity.

Specific examples of compounds include compounds having GLP-1 and/or GLP-2- receptor agonist activity.

Specific examples of compounds include compounds that have higher GLP-2 receptor agonist activity compared to GLP-1 receptor agonist activity.

The compounds of the invention may be used in a pharmaceutical composition comprising a compound of the invention and a pharmaceutically acceptable excipient.

The compounds of the invention may be used in medicine.

The compounds of the invention may be used in the treatment of disorders associated with GLP receptors.

The compounds of the invention may be used in the treatment of disorders associated with the GLP-1 and/or GLP-2 receptor.

The present invention provides the use of a GLP-2/GLP-1 analogue compound for the preparation of a medicament for treating gastrointestinal and metabolic diseases. GLP-2/GLP-1 analogues as defined herein may be useful for promoting intestinal recovery and nutritional status of patients with malabsorption disorders, intestinal failure, intestinal insufficiency, diarrheal diseases and chronic inflammatory bowel disorders. In addition, therapeutic treatment with a GLP-2/GLP-1 analogue may improve mucosal barrier function, ameliorate gut inflammation and reduce intestinal permeability which could improve symptoms in patients with inflammatory disorders, celiac disease, congenital and acquired digestion and malabsorption syndromes, chronic diarrhoeal diseases, conditions caused by mucosal damage (e.g. cancer treatment). A GLP-2/GLP-1 analogue of the present invention is anticipated to restore glycemic control and insulin sensitivity. This could be beneficial for the management of hyperglycemia during enteral and parenteral nutrition therapy in patients with intestinal failure, insufficiency or malabsorption disorders.

In a further aspect, the present invention provides a methods of treating one of the group consisting of gastrointestinal injury, diarrheal diseases, intestinal insufficiency, intestinal failure, acid-induced intestinal injury, arginine deficiency, obesity, celiac disease, chemotherapy-induced enteritis, diabetes, obesity, fat malabsorption, steatorrhea, autoimmune diseases, food allergies, gastric ulcers, gastrointestinal barrier disorders, Parkinson's disease, sepsis, bacterial peritonitis, inflammatory bowel disease, chemotherapy-associated tissue damage, bowel trauma, bowel ischemia, mesenteric ischemia, short bowel syndrome, malnutrition, necrotizing enterocolitis, necrotizing pancreatitis, neonatal feeding intolerance, NSAID-induced gastrointestinal damage, nutritional insufficiency, total parenteral nutrition damage to gastrointestinal tract, neonatal nutritional insufficiency, radiation-induced enteritis, radiation-induced injury to the intestines, mucositis, pouchitis, ischemia, obesity, type 2 diabetes, non-alcoholic fatty liver disease (NAFLD), nonalcoholic steatohepatitis (NASH), insulin resistance, hyperglycemia, insulin resistance, glucose intolerance.

Specifically, it is suggested that congenital diarrheal diseases which are characterised by severe diarrhoea, fluid and electrolyte loss, malabsorption and impairment of nutritional transport could be ameliorated by treatment with GLP-2 /and GLP-1 analogues of this invention. In particular, Tufting enteropathy is a condition associated with disrupted villus morphological architecture, that results in impaired nutrient absorption and enhanced intestinal permeability. Agents that can improve fluid and nutritional absorption, as well as correct the gut barrier impairment may offer value in promoting early weaning from parenteral nutrition.

Other examples of congenital diarrheal diseases that may be treated with a peptide of the invention includes brush border enzyme deficiencies (congenital lactase deficiency, congenital sucrase-isomaltase deficiency, congenital maltase-glucoamylase-deficiency), defects of membrane carriers (glucose-galactose-malabsorption, fructose malabsorption, Acrodermatitis enteropathica, Congenital chloride / sodium diarrhoea, Primary biliary malabsorption, cystic fibrosis), lipid/lipoprotein metabolism defects (chylomicron retention disease, abetalipoproteinemia), defects of enterocyte differentiation or cellular polarisation (Microvillous atrophy, Tufting enteropathy, Trichohepatoenteric syndrome,) and defects of enteroendocrine cells (Congenital malabsorptive diarrhoea, anendocrinosis, protein-convertase ⅓ deficiency).

The compounds of the invention may be used in the treatment of Tufting enteropathy.

### Definitions

In this application, the following definitions apply, unless indicated otherwise.

The term “alkyl”, “aryl”, “halogen”, “alkoxy”, “cycloalkyl”, “heterocyclyl” and “heteroaryl” are used in their conventional sense (e.g. as defined in the IUPAC Gold Book) unless indicated otherwise.

The term “treatment”, in relation to the uses of any of the compounds described herein, including those of the formula (1a) and (1b), is used to describe any form of intervention where a compound is administered to a subject suffering from, or at risk of suffering from, or potentially at risk of suffering from the disease or disorder in question. Thus, the term “treatment” covers both preventative (prophylactic) treatment and treatment where measurable or detectable symptoms of the disease or disorder are being displayed.

The term “effective therapeutic amount” as used herein (for example in relation to methods of treatment of a disorder, disease or condition) refers to an amount of the compound which is effective to produce a desired therapeutic effect. For example, if the condition is pain, then the effective therapeutic amount is an amount sufficient to provide a desired level of pain relief. The desired level of pain relief may be, for example, complete removal of the pain or a reduction in the severity of the pain.

To the extent that any of the compounds described have chiral centres, the present invention extends to all optical isomers of such compounds, whether in the form of racemates or resolved enantiomers. The invention described herein relates to all crystal forms, solvates and hydrates of any of the disclosed compounds however so prepared. To the extent that any of the compounds disclosed herein have acid or basic centres such as carboxylates or amino groups, then all salt forms of said compounds are included herein. In the case of pharmaceutical uses, the salt should be seen as being a pharmaceutically acceptable salt.

Salts or pharmaceutically acceptable salts that may be mentioned include acid addition salts and base addition salts. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Examples of pharmaceutically acceptable salts include acid addition salts derived from mineral acids and organic acids, and salts derived from metals such as sodium, magnesium, potassium and calcium.

Examples of acid addition salts include acid addition salts formed with acetic, 2,2-dichloroacetic, adipic, alginic, aryl sulfonic acids (e.g. benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (+)-DL-lactic), lactobionic, maleic, malic (e.g. (−)-L-malic), malonic, (+)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g. (+)-L-tartaric), thiocyanic, undecylenic and valeric acids.

Also encompassed are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulfoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGA), differential scanning calorimetry (DSC) and X-ray crystallography.

The solvates can be stoichiometric or non-stoichiometric solvates. Particular solvates may be hydrates, and examples of hydrates include hemihydrates, monohydrates and dihydrates. For a more detailed discussion of solvates and the methods used to make and characterise them, see Bryn et al, Solid-State Chemistry of Drugs, Second Edition, published by SSCI, Inc of West Lafayette, IN, USA, 1999, ISBN 0-967-06710-3.

The term “pharmaceutical composition” in the context of this invention means a composition comprising an active agent and comprising additionally one or more pharmaceutically acceptable carriers. The composition may further contain ingredients selected from, for example, diluents, adjuvants, excipients, vehicles, preserving agents, fillers, disintegrating agents, wetting agents, emulsifying agents, suspending agents, sweetening agents, flavouring agents, perfuming agents, antibacterial agents, antifungal agents, lubricating agents and dispersing agents, depending on the nature of the mode of administration and dosage forms. The compositions may take the form, for example, of tablets, dragees, powders, elixirs, syrups, liquid preparations including suspensions, sprays, inhalants, tablets, lozenges, emulsions, solutions, cachets, granules, capsules and suppositories, as well as liquid preparations for injections, including liposome preparations.

The compounds of the invention may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. In an analogous manner, a reference to a particular functional group also includes within its scope isotopic variations, unless the context indicates otherwise. For example, a reference to an alkyl group such as an ethyl group or an alkoxy group such as a methoxy group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group) or a methoxy group in which all three hydrogen atoms are in the deuterium isotopic form (a trideuteromethoxy group). The isotopes may be radioactive or non-radioactive.

Therapeutic dosages may be varied depending upon the requirements of the patient, the severity of the condition being treated, and the compound being employed. Determination of the proper dosage for a particular situation is within the skill of the art. Generally, treatment is initiated with the smaller dosages which are less than the optimum dose of the compound. Thereafter the dosage is increased by small increments until the optimum effect under the circumstances is reached. For convenience, the total daily dosage may be divided and administered in portions during the day if desired.

The magnitude of an effective dose of a compound will, of course, vary with the nature of the severity of the condition to be treated and with the particular compound and its route of administration. The selection of appropriate dosages is within the ability of one of ordinary skill in this art, without undue burden. In general, the daily dose range may be from about 10 μg to about 30 mg per kg body weight of a human and non-human animal, preferably from about 50 μg to about 30 mg per kg of body weight of a human and non-human animal, for example from about 50 μg to about 10 mg per kg of body weight of a human and non-human animal, for example from about 100 μg to about 30 mg per kg of body weight of a human and non-human animal, for example from about 100 μg to about 10 mg per kg of body weight of a human and non-human animal and most preferably from about 100 μg to about 1 mg per kg of body weight of a human and non-human animal.

### Pharmaceutical Formulations

While it is possible for the active compound to be administered alone, it is preferable to present it as a pharmaceutical composition (e.g. formulation).

Accordingly, in another embodiment of the invention, there is provided a pharmaceutical composition comprising at least one compound of the formula (1a) and (1b) as defined above together with at least one pharmaceutically acceptable excipient.

The composition may be a tablet composition.

The composition may be a capsule composition.

The composition may be a composition suitable for injection. The injection may be intra-venous (IV) or subcutaneous. The composition may be supplied in a sterile buffer solution or as a solid which can be suspended or dissolved in sterile buffer for injection.

The pharmaceutically acceptable excipient(s) can be selected from, for example, carriers (e.g. a solid, liquid or semi-solid carrier), adjuvants, diluents (e.g solid diluents such as fillers or bulking agents; and liquid diluents such as solvents and co-solvents), granulating agents, binders, flow aids, coating agents, release-controlling agents (e.g. release retarding or delaying polymers or waxes), binding agents, disintegrants, buffering agents, lubricants, preservatives, anti-fungal and antibacterial agents, antioxidants, buffering agents, tonicity-adjusting agents, thickening agents, flavouring agents, sweeteners, pigments, plasticizers, taste masking agents, stabilisers or any other excipients conventionally used in pharmaceutical compositions.

The term “pharmaceutically acceptable” as used herein means compounds, materials, compositions, and/or dosage forms which are, within the scope of sound medical judgment, suitable for use in contact with the tissues of a subject (e.g. a human subject) without excessive toxicity, irritation, allergic response, or other problem or complication, commensurate with a reasonable benefit/risk ratio. Each excipient must also be “acceptable” in the sense of being compatible with the other ingredients of the formulation.

Pharmaceutical compositions containing compounds of the formula (1a) and (1b) can be formulated in accordance with known techniques, see for example, Remington's Pharmaceutical Sciences, Mack Publishing Company, Easton, PA, USA.

The pharmaceutical compositions can be in any form suitable for oral, parenteral, topical, intranasal, intrabronchial, sublingual, ophthalmic, otic, rectal, intra-vaginal, or transdermal administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

Tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract.

The pharmaceutical compositions typically comprise from approximately 1% (w/w) to approximately 95%, preferably % (w/w) active ingredient and from 99% (w/w) to 5% (w/w) of a pharmaceutically acceptable excipient (for example as defined above) or combination of such excipients. Preferably, the compositions comprise from approximately 20% (w/w) to approximately 90% (w/w) active ingredient and from 80% (w/w) to 10% of a pharmaceutically excipient or combination of excipients. The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, pre-filled syringes, dragées, powders, tablets or capsules.

Tablets and capsules may contain, for example, 0-20% disintegrants, 0-5% lubricants, 0-5% flow aids and/or 0-99% (w/w) fillers/ or bulking agents (depending on drug dose). They may also contain 0-10% (w/w) polymer binders, 0-5% (w/w) antioxidants, 0-5% (w/w) pigments. Slow release tablets would in addition typically contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

The pharmaceutical formulations may be presented to a patient in “patient packs” containing an entire course of treatment in a single package, usually a blister pack.

The compounds of the formula (1a) and (1b) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 milligrams to 1 gram, of active compound.

The active compound will be administered to a patient in need thereof (for example a human or animal patient) in an amount sufficient to achieve the desired therapeutic effect (effective amount). The precise amounts of compound administered may be determined by a supervising physician in accordance with standard procedures.

### Biological Activity

The in vitro GLP-2 assay results for compounds illustrated in Table 1 were in the range from about 0.001 nM to about 1 nM. The GLP-2 analogues of the invention demonstrate activity at both GLP-2 and GLP-1 receptors, with greater activity demonstrated at the GLP-2 receptor.

### EXAMPLES

The invention will now be illustrated, but not limited, by reference to the specific embodiments described in the following examples.

### Examples 1 to 81

The compounds of Examples 1 to 117 shown in Table 1 and Table 1a below have been prepared. Their LCMS properties and the methods used to prepare them are set out in Table 2 and Table 2a. The starting materials for each of the Examples are commercial unless indicated otherwise.

TABLE 1

| EX | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| 1 | — | CAP | TET | GLY | SER | PHE | SER | Cyclo | GLU | NLE | D- | Cyclo | ILE | LEU |
| | | | | | | | | LYS | | | PHE | ASP | | |
| 2 | — | CAP | TET | GLY | SER | PHE | SER | Cyclo | GLU | NLE | D- | Cyclo | ILE | LEU |
| | | | | | | | | LYS | | | PHE | ASP | | |
| 3 | — | CAP | TET | Cyclo | SER | PHE | SER | Cyclo | GLU | NLE | D- | Cyclo | ILE | LEU |
| | | | | LYS | | | | LYS | | | PHE | GLU | | |
| 4 | HIS | AIB | ASP | GLY | SER | PHE | SER | Cyclo | GLU | NLE | D- | Cyclo | ILE | LEU |
| | | | | | | | | LYS | | | PHE | ASP | | |
| 5 | HIS | AIB | ASP | Cyclo | SER | PHE | SER | Cyclo | GLU | NLE | D- | Cyclo | ILE | LEU |
| | | | | LYS | | | | LYS | | | PHE | GLU | | |
| 6 | — | CAP | TET | GLY | SER | PHE | SER | Cyclo | GLU | NLE | D- | Cyclo | ILE | LEU |
| | | | | | | | | LYS | | | PHE | ASP | | |
| 7 | — | CAP | TET | GLY | SER | PHE | SER | Cyclo | GLU | NLE | D- | Cyclo | ILE | LEU |
| | | | | | | | | LYS | | | PHE | ASP | | |
| 8 | — | CAP | TET | D- | SER | PHE | SER | Cyclo | GLU | NLE | D- | Cyclo | α- | LEU |
| | | | | ALA | | | | LYS | | | PHE | ASP | MeLEU | |
| 9 | — | CAP | TET | GLY | SER | PHE | SER | Cyclo | GLU | NLE | D- | Cyclo | ILE | LEU |
| | | | | | | | | LYS | | | PHE | ASP | | |
| 10 | — | CAP | TET | GLY | SER | PHE | SER | Cyclo | GLU | NLE | D- | Cyclo | ILE | LEU |
| | | | | | | | | LYS | | | PHE | ASP | | |
| 11 | — | CAP | TET | GLY | SER | PHE | SER | Cyclo | GLU | NLE | D- | Cyclo | α- | LEU |
| | | | | | | | | LYS | | | PHE | ASP | MeLEU | |
| 12 | — | CAP | TET | GLY | SER | PHE | SER | Cyclo | GLU | NLE | D- | Cyclo | ILE | LEU |
| | | | | | | | | LYS | | | PHE | ASP | | |
| 13 | — | CAP | TET | GLY | SER | PHE | SER | Cyclo | GLU | NLE | D- | Cyclo | ILE | LEU |
| | | | | | | | | LYS | | | PHE | ASP | | |
| 14 | — | CAP | TET | GLY | SER | PHE | SER | Cyclo | GLU | NLE | D- | Cyclo | ILE | LEU |
| | | | | | | | | LYS | | | PHE | ASP | | |
| 15 | — | CAP | TET | D- | SER | 2-F, α- | SER | Cyclo | GLU | NLE | D- | Cyclo | ILE | LEU |
| | | | | ALA | | MePHE | | LYS | | | PHE | ASP | | |
| 16 | — | CAP | TET | GLY | SER | PHE | SER | Cyclo | GLU | NLE | D- | Cyclo | ILE | LEU |
| | | | | | | | | LYS | | | PHE | ASP | | |
| 17 | — | CAP | TET | Cyclo | SER | PHE | SER | Cyclo | GLU | NLE | D- | THR | α- | LEU |
| | | | | LYS | | | | ASP | | | PHE | | MeLEU | |
| 18 | HIS | AIB | ASP | GLY | SER | 2-F, α- | SER | Cyclo | GLU | NLE | D- | Cyclo | ILE | LEU |
| | | | | | | MePHE | | LYS | | | PHE | ASP | | |
| 19 | HIS | AIB | ASP | D- | SER | 2-F, α- | SER | Cyclo | GLU | NLE | D- | Cyclo | ILE | LEU |
| | | | | ALA | | MePHE | | LYS | | | PHE | ASP | | |
| 20 | HIS | AIB | ASP | GLY | SER | 2-F, α- | SER | Cyclo | GLU | NLE | D- | Cyclo | ILE | LEU |
| | | | | | | MePHE | | LYS | | | PHE | ASP | | |
| 21 | HIS | AIB | ASP | Cyclo | SER | PHE | SER | Cyclo | GLU | NLE | D- | THR | α- | LEU |
| | | | | LYS | | | | ASP | | | PHE | | MeLEU | |
| 22 | — | CAP | TET | GLY | SER | PHE | SER | Cyclo | GLU | NLE | D- | Cyclo | ILE | LEU |
| | | | | | | | | LYS | | | PHE | ASP | | |
| 23 | — | CAP | TET | GLY | SER | PHE | SER | Cyclo | GLU | NLE | D- | Cyclo | ILE | LEU |
| | | | | | | | | LYS | | | PHE | ASP | | |
| 24 | — | CAP | TET | D- | SER | PHE | SER | Cyclo | GLU | NLE | D- | Cyclo | α- | LEU |
| | | | | ALA | | | | LYS | | | PHE | ASP | MeLEU | |
| 25 | — | CAP | ASP | D- | SER | 2-F, α- | SER | Cyclo | GLU | NLE | D- | Cyclo | α- | LEU |
| | | | | ALA | | MePHE | | LYS | | | PHE | ASP | MeLEU | |
| 26 | — | CAP | ASP | D- | SER | 2-F, α- | SER | Cyclo | GLU | NLE | D- | Cyclo | ILE | LEU |
| | | | | ALA | | MePHE | | LYS | | | PHE | ASP | | |
| 27 | HIS | AIB | GLN | Cyclo | SER | PHE | SER | Cyclo | GLU | NLE | D- | THR | α- | LEU |
| | | | | LYS | | | | ASP | | | PHE | | MeLEU | |
| 28 | HIS | AIB | GLU | Cyclo | SER | PHE | SER | Cyclo | GLU | NLE | D- | THR | α- | LEU |
| | | | | LYS | | | | ASP | | | PHE | | MeLEU | |
| 29 | HIS | AIB | ASP | Cyclo | SER | PHE | SER | Cyclo | GLU | NLE | D- | THR | α- | LEU |
| | | | | LYS | | | | ASP | | | PHE | | MeLEU | |
| 30 | HIS | AIB | ASP | Cyclo | SER | PHE | SER | Cyclo | GLU | NLE | D- | THR | α- | LEU |
| | | | | LYS | | | | ASP | | | PHE | | MeLEU | |
| 31 | HIS | AIB | ASP | Cyclo | SER | 2-F, α- | SER | Cyclo | GLU | NLE | D- | THR | α- | LEU |
| | | | | LYS | | MePHE | | ASP | | | PHE | | MeLEU | |
| 32 | HIS | AIB | GLU | Cyclo | SER | 2-F, α- | SER | Cyclo | GLU | NLE | D- | THR | α- | LEU |
| | | | | LYS | | MePHE | | ASP | | | PHE | | MeLEU | |
| 33 | — | CAP | ASP | Cyclo | SER | 2-F, α- | SER | Cyclo | GLU | NLE | D- | THR | α- | LEU |
| | | | | LYS | | MePHE | | ASP | | | PHE | | MeLEU | |
| 34 | — | CAP | ASP | Cyclo | SER | 2-F, α- | SER | Cyclo | GLU | NLE | D- | THR | α- | LEU |
| | | | | LYS | | MePHE | | ASP | | | PHE | | MeLEU | |
| 35 | HIS | AIB | ASP | Cyclo | SER | 2-F, α- | SER | Cyclo | GLU | NLE | D- | THR | α- | LEU |
| | | | | LYS | | MePHE | | ASP | | | PHE | | MeLEU | |
| 36 | HIS | AIB | ASP | Cyclo | SER | PHE | SER | Cyclo | GLU | NLE | D-α- | THR | α- | LEU |
| | | | | LYS | | | | ASP | | | MePHE | | MeLEU | |
| 37 | HIS | AIB | ASP | Cyclo | SER | PHE | SER | Cyclo | GLU | NLE | D- | THR | α- | LEU |
| | | | | LYS | | | | ASP | | | PHE | | MeLEU | |
| 38 | | CAP | TET | D- | SER | PHE | SER | Cyclo | GLU | NLE | D- | Cyclo | ILE | LEU |
| | | | | ALA | | | | LYS | | | PHE | ASP | | |
| 39 | HIS | AIB | ASP | Cyclo | SER | PHE | SER | Cyclo | GLU | NLE | D- | Cyclo | ILE | LEU |
| | | | | LYS | | | | ASP | | | PHE | GLU | | |
| 40 | — | CAP | TET | D- | SER | PHE | SER | Cyclo | GLU | NLE | D- | Cyclo | ILE | LEU |
| | | | | ALA | | | | LYS | | | PHE | ASP | | |
| 41 | — | CAP | TET | Cyclo | SER | PHE | SER | Cyclo | GLU | NLE | D- | Cyclo | ILE | LEU |
| | | | | LYS | | | | ASP | | | PHE | GLU | | |
| 42 | — | CAP | TET | Cyclo | SER | PHE | SER | Cyclo | GLU | NLE | D- | Cyclo | ILE | LEU |
| | | | | LYS | | | | ASP | | | PHE | LYS | | |
| 43 | — | CAP | TET | D- | SER | 2-F, α- | SER | Cyclo | GLU | NLE | D- | Cyclo | ILE | LEU |
| | | | | ALA | | MePHE | | LYS | | | PHE | ASP | | |
| 44 | — | CAP | TET | Cyclo | SER | PHE | SER | Cyclo | GLU | NLE | D- | THR | α- | LEU |
| | | | | LYS | | | | ASP | | | PHE | | MeLEU | |
| 45 | — | CAP | TET | Cyclo | SER | PHE | SER | Cyclo | GLU | NLE | D- | THR | α- | LEU |
| | | | | LYS | | | | ASP | | | PHE | | MeLEU | |
| 46 | HIS | AIB | ASP | Cyclo | SER | PHE | SER | Cyclo | GLU | NLE | D- | THR | α- | LEU |
| | | | | LYS | | | | ASP | | | PHE | | MeLEU | |
| 47 | HIS | AIB | ASP | Cyclo | SER | PHE | SER | Cyclo | GLU | NLE | D- | Cyclo | ILE | LEU |
| | | | | LYS | | | | ASP | | | PHE | LYS | | |
| 48 | HIS | AIB | ASP | GLY | SER | 2-F, α- | SER | ASP | GLU | NLE | D- | Cyclo | ILE | LEU |
| | | | | | | MePHE | | | | | PHE | GLU | | |
| 49 | HIS | AIB | ASP | GLY | SER | 2-F, α- | SER | ASP | GLU | NLE | D- | Cyclo | ILE | LEU |
| | | | | | | MePHE | | | | | PHE | GLU | | |
| 50 | — | CAP | ASP | D- | SER | 2-F, α- | SER | Cyclo | GLU | NLE | D- | Cyclo | α- | LEU |
| | | | | ALA | | MePHE | | LYS | | | PHE | ASP | MeLEU | |
| 51 | — | CAP | ASP | D- | SER | 2-F, α- | SER | Cyclo | GLU | NLE | D- | Cyclo | ILE | LEU |
| | | | | ALA | | MePHE | | LYS | | | PHE | ASP | | |
| 52 | — | CAP | TET | GLY | SER | 2-F, α- | SER | ASP | GLU | NLE | D- | Cyclo | ILE | LEU |
| | | | | | | MePHE | | | | | PHE | GLU | | |
| 53 | — | CAP | TET | GLY | SER | 2-F, α- | SER | ASP | GLU | NLE | D- | Cyclo | ILE | LEU |
| | | | | | | MePHE | | | | | PHE | GLU | | |
| 54 | — | CAP | GLN | D- | SER | 2-F, α- | SER | Cyclo | GLU | NLE | D- | Cyclo | ILE | LEU |
| | | | | ALA | | MePHE | | LYS | | | PHE | ASP | | |
| 55 | — | CAP | TET | D- | SER | 2-F, α- | SER | Cyclo | GLU | NLE | D- | Cyclo | ILE | LEU |
| | | | | ALA | | MePHE | | LYS | | | PHE | ASP | | |
| 56 | — | CAP | TET | D- | SER | 2-F, α- | SER | Cyclo | GLU | NLE | D- | Cyclo | α- | LEU |
| | | | | ALA | | MePHE | | LYS | | | PHE | ASP | MeLEU | |
| 57 | — | CAP | TET | D- | SER | 2-F, α- | SER | Cyclo | GLU | NLE | D- | Cyclo | α- | LEU |
| | | | | ALA | | MePHE | | LYS | | | PHE | ASP | MeLEU | |
| 58 | — | CAP | ASP | D- | SER | 2-F, α- | SER | Cyclo | GLU | NLE | D- | Cyclo | α- | LEU |
| | | | | ALA | | MePHE | | LYS | | | PHE | ASP | MeLEU | |
| 59 | HIS | AIB | ASP | D- | SER | 2-F, α- | SER | Cyclo | GLU | NLE | D- | Cyclo | ILE | LEU |
| | | | | ALA | | MePHE | | LYS | | | PHE | ASP | | |
| 60 | HIS | AIB | ASP | D- | SER | 2-F, α- | SER | Cyclo | GLU | NLE | D- | Cyclo | α- | LEU |
| | | | | ALA | | MePHE | | LYS | | | PHE | ASP | MeLEU | |
| 61 | HIS | AIB | ASP | D- | SER | 2-F, α- | SER | Cyclo | GLU | NLE | D- | Cyclo | ILE | LEU |
| | | | | ALA | | MePHE | | LYS | | | PHE | ASP | | |
| 62 | HIS | AIB | ASP | D- | SER | 2-F, α- | SER | Cyclo | GLU | NLE | D- | Cyclo | α- | LEU |
| | | | | ALA | | MePHE | | LYS | | | PHE | ASP | MeLEU | |
| 63 | HIS | AIB | GLN | Cyclo | SER | PHE | SER | Cyclo | GLU | NLE | D- | THR | α- | LEU |
| | | | | LYS | | | | ASP | | | PHE | | MeLEU | |
| 64 | HIS | AIB | ASP | Cyclo | SER | 2-F, α- | SER | Cyclo | GLU | NLE | D- | THR | α- | LEU |
| | | | | LYS | | MePHE | | ASP | | | PHE | | MeLEU | |
| 65 | — | CAP | ASP | Cyclo | SER | 2-F, α- | SER | Cyclo | GLU | NLE | D- | THR | α- | LEU |
| | | | | LYS | | MePHE | | ASP | | | PHE | | MeLEU | |
| 66 | — | CAP | ASP | Cyclo | SER | 2-F, α- | SER | Cyclo | GLU | NLE | D- | THR | α- | LEU |
| | | | | LYS | | MePHE | | ASP | | | PHE | | MeLEU | |
| 67 | HIS | AIB | ASP | Cyclo | SER | 2-F, α- | SER | Cyclo | GLU | NLE | D- | THR | α- | LEU |
| | | | | LYS | | MePHE | | ASP | | | PHE | | MeLEU | |
| 68 | — | CAP | ASP | Cyclo | SER | PHE | SER | Cyclo | GLU | NLE | D- | THR | α- | LEU |
| | | | | LYS | | | | ASP | | | PHE | | MeLEU | |
| 69 | — | CAP | GLN | Cyclo | SER | PHE | SER | Cyclo | GLU | NLE | D- | THR | α- | LEU |
| | | | | LYS | | | | ASP | | | PHE | | MeLEU | |
| 70 | — | CAP | GLN | Cyclo | SER | 2-F, α- | SER | Cyclo | GLU | NLE | D- | THR | α- | LEU |
| | | | | LYS | | MePHE | | ASP | | | PHE | | MeLEU | |
| 71 | — | CAP | TET | Cyclo | SER | 2-F, α- | SER | Cyclo | GLU | NLE | D- | THR | α- | LEU |
| | | | | LYS | | MePHE | | ASP | | | PHE | | MeLEU | |
| 72 | — | CAP | TET | Cyclo | SER | 2-F, α- | SER | Cyclo | GLU | NLE | D- | THR | α- | LEU |
| | | | | LYS | | MePHE | | ASP | | | PHE | | MeLEU | |
| 73 | — | CAP | TET | Cyclo | SER | PHE | SER | Cyclo | GLU | NLE | D- | THR | α- | LEU |
| | | | | LYS | | | | ASP | | | PHE | | MeLEU | |
| 74 | HIS | AIB | ASP | Cyclo | SER | PHE | SER | Cyclo | GLU | NLE | D- | Cyclo | ILE | LEU |
| | | | | GLU | | | | LYS | | | PHE | GLU | | |
| 75 | HIS | AIB | ASP | Cyclo | SER | PHE | SER | Cyclo | GLU | NLE | D- | Cyclo | ILE | LEU |
| | | | | GLU | | | | LYS | | | PHE | GLU | | |
| 76 | HIS | AIB | ASP | GLY | SER | PHE | Cyclo | ASP | GLU | NLE | Cyclo | Cyclo | ILE | LEU |
| | | | | | | | GLU | | | | LYS | GLU | | |
| 77 | HIS | AIB | ASP | GLY | SER | PHE | Cyclo | ASP | GLU | NLE | Cyclo | Cyclo | ILE | LEU |
| | | | | | | | GLU | | | | LYS | GLU | | |
| 78 | — | CAP | TET | Cyclo | SER | PHE | SER | Cyclo | GLU | NLE | D- | Cyclo | ILE | LEU |
| | | | | GLU | | | | LYS | | | PHE | GLU | | |
| 79 | — | CAP | TET | Cyclo | SER | PHE | SER | Cyclo | GLU | NLE | D- | Cyclo | ILE | LEU |
| | | | | GLU | | | | LYS | | | PHE | GLU | | |
| 80 | — | CAP | TET | GLY | SER | PHE | Cyclo | ASP | GLU | NLE | Cyclo | Cyclo | ILE | LEU |
| | | | | | | | GLU | | | | LYS | GLU | | |
| 81 | — | CAP | TET | GLY | SER | PHE | Cyclo | ASP | GLU | NLE | Cyclo | Cyclo | ILE | LEU |
| | | | | | | | GLU | | | | LYS | GLU | | |
| | | | EX | 15 | 16 | 17 | 18 | 19 | 20 | 21 | 22 | 23 | 24 | 25 |
| | | | 1 | ASP | Cyclo | LYS | ALA | ALA | Cyclo | ASP | PHE | ILE | ASN | TRP |
| | | | | | ASP | | | | LYS | | | | | |
| | | | 2 | ASP | Cyclo | LYS | ALA | ALA | Cyclo | ASP | PHE | ILE | ASN | TRP |
| | | | | | GLU | | | | LYS | | | | | |
| | | | 3 | ASP | Cyclo | LYS | ALA | ALA | AIB | ASP | PHE | ILE | Cyclo | TRP |
| | | | | | LYS | | | | | | | | GLU | |
| | | | 4 | ASP | Cyclo | LYS | ALA | ALA | Cyclo | ASP | PHE | ILE | ASN | TRP |
| | | | | | GLU | | | | LYS | | | | | |
| | | | 5 | ASP | Cyclo | LYS | ALA | ALA | AIB | ASP | PHE | ILE | Cyclo | TRP |
| | | | | | LYS | | | | | | | | GLU | |
| | | | 6 | ACPC | Cyclo | LYS | ALA | ALA | Cyclo | ASP | PHE | ILE | ACPC | TRP |
| | | | | | GLU | | | | LYS | | | | | |
| | | | 7 | ACPC | Cyclo | LYS | ALA | ALA | Cyclo | ASP | PHE | ILE | ACPC | TRP |
| | | | | | GLU | | | | LYS | | | | | |
| | | | 8 | ASP | Cyclo | LYS | ALA | ALA | Cyclo | ASP | PHE | ILE | ASN | TRP |
| | | | | | GLU | | | | LYS | | | | | |
| | | | 9 | ASP | Cyclo | LYS- | ALA | ALA | Cyclo | ASP | PHE | ILE | ASN | TRP |
| | | | | | GLU | yGlu- | | | LYS | | | | | |
| | | | | | | 2xOEG | | | | | | | | |
| | | | | | | C18 | | | | | | | | |
| | | | | | | dlacld | | | | | | | | |
| | | | 10 | ASP | Cyclo | LYS- | ALA | ALA | Cyclo | ASP | PHE | ILE | ASN | TRP |
| | | | | | GLU | yGlu- | | | LYS | | | | | |
| | | | | | | 2xOEG | | | | | | | | |
| | | | | | | C18 | | | | | | | | |
| | | | | | | diacid | | | | | | | | |
| | | | 11 | ASP | Cyclo | LYS | ALA | ALA | Cyclo | ASP | PHE | ILE | ASN | TRP |
| | | | | | GLU | | | | LYS | | | | | |
| | | | 12 | ASP | Cyclo | LYS | AIB | ALA | Cyclo | ASP | PHE | ILE | ASN | TRP |
| | | | | | GLU | | | | LYS | | | | | |
| | | | 13 | ASP | Cyclo | LYS | ALA | ALA | Cyclo | ASP | PHE | ILE | ASN | TRP |
| | | | | | GLU | | | | LYS | | | | | |
| | | | 14 | ASP | Cyclo | LYS | ALA | ALA | Cyclo | ASP | PHE | ILE | ASN | TRP |
| | | | | | GLU | | | | LYS | | | | | |
| | | | 15 | ASP | Cyclo | LYS | ALA | ALA | Cyclo | ASP | PHE | ILE | ASN | TRP |
| | | | | | GLU | | | | LYS | | | | | |
| | | | 16 | ASP | Cyclo | LYS | AIB | AIB | Cyclo | ASP | PHE | ILE | ASN | TRP |
| | | | | | GLU | | | | LYS | | | | | |
| | | | 17 | ASP | Cyclo | LYS | ALA | ALA | Cyclo | ASP | PHE | ILE | Cyclo | TRP |
| | | | | | GLU | | | | LYS | | | | GLU | |
| | | | 18 | ASP | Cyclo | LYS | ALA | ALA | Cyclo | ASP | PHE | ILE | ASN | TRP |
| | | | | | GLU | | | | LYS | | | | | |
| | | | 19 | ASP | Cyclo | LYS | ALA | ALA | Cyclo | ASP | PHE | ILE | ASN | TRP |
| | | | | | GLU | | | | LYS | | | | | |
| | | | 20 | ASP | Cyclo | LYS- | ALA | ALA | Cyclo | ASP | PHE | ILE | ASN | TRP |
| | | | | | GLU | yGlu- | | | LYS | | | | | |
| | | | | | | 2xOEG | | | | | | | | |
| | | | | | | C18 | | | | | | | | |
| | | | | | | diacid | | | | | | | | |
| | | | 21 | ASP | Cyclo | LYS | ALA | ALA | Cyclo | ASP | PHE | ILE | Cyclo | TRP |
| | | | | | GLU | | | | LYS | | | | GLU | |
| | | | 22 | ASP | Cyclo | LYS | ALA | ALA | Cyclo | ASP | PHE | ILE | ASN | TRP |
| | | | | | GLU | | | | LYS | | | | | |
| | | | 23 | ASP | Cyclo | Alloc | ALA | ALA | Cyclo | ASP | PHE | ILE | ASN | TRP |
| | | | | | GLU | Lys | | | LYS | | | | | |
| | | | 24 | ASP | Cyclo | LYS | ALA | ALA | Cyclo | ASP | PHE | ILE | ASN | TRP |
| | | | | | GLU | | | | LYS | | | | | |
| | | | 25 | ASP | Cyclo | LYS | ALA | ALA | Cyclo | ASP | PHE | ILE | ASN | TRP |
| | | | | | GLU | | | | LYS | | | | | |
| | | | 26 | ASP | Cyclo | LYS | ALA | ALA | Cyclo | ASP | PHE | ILE | ASN | TRP |
| | | | | | GLU | | | | LYS | | | | | |
| | | | 27 | ASP | Cyclo | LYS | ALA | ALA | Cyclo | ASP | PHE | ILE | Cyclo | TRP |
| | | | | | GLU | | | | LYS | | | | GLU | |
| | | | 28 | ASP | Cyclo | LYS | ALA | ALA | Cyclo | ASP | PHE | ILE | Cyclo | TRP |
| | | | | | GLU | | | | LYS | | | | GLU | |
| | | | 29 | ASP | Cyclo | LYS | ALA | ALA | Cyclo | ASP | PHE | ILE | Cyclo | TRP |
| | | | | | GLU | | | | LYS | | | | GLU | |
| | | | 30 | ASP | Cyclo | LYS | ALA | ALA | Cyclo | ASP | PHE | ILE | Cyclo | TRP |
| | | | | | GLU | | | | LYS | | | | GLU | |
| | | | 31 | ASP | Cyclo | LYS | ALA | ALA | Cyclo | ASP | PHE | ILE | Cyclo | TRP |
| | | | | | GLU | | | | LYS | | | | GLU | |
| | | | 32 | ASP | Cyclo | LYS | ALA | ALA | Cyclo | ASP | PHE | ILE | Cyclo | TRP |
| | | | | | GLU | | | | LYS | | | | GLU | |
| | | | 33 | ASP | Cyclo | LYS | ALA | ALA | Cyclo | ASP | PHE | ILE | Cyclo | TRP |
| | | | | | GLU | | | | LYS | | | | GLU | |
| | | | 34 | ASP | Cyclo | LYS- | ALA | ALA | Cyclo | ASP | PHE | ILE | Cyclo | TRP |
| | | | | | GLU | yGlu- | | | LYS | | | | GLU | |
| | | | | | | 2xOEG | | | | | | | | |
| | | | | | | C18 | | | | | | | | |
| | | | | | | diacid | | | | | | | | |
| | | | 35 | ASP | Cyclo | LYS- | ALA | ALA | Cyclo | ASP | PHE | ILE | Cyclo | TRP |
| | | | | | GLU | yGlu- | | | LYS | | | | GLU | |
| | | | | | | 2xOEG | | | | | | | | |
| | | | | | | C18 | | | | | | | | |
| | | | | | | diacid | | | | | | | | |
| | | | 36 | ASP | Cyclo | LYS | ALA | ALA | Cyclo | ASP | PHE | ILE | Cyclo | TRP |
| | | | | | GLU | | | | LYS | | | | GLU | |
| | | | 37 | ASP | Cyclo | LYS | ALA | ALA | Cyclo | ASP | PHE | ILE | Cyclo | TRP |
| | | | | | GLU | | | | LYS | | | | GLU | |
| | | | 38 | ASP | Cyclo | LYS | ALA | ALA | Cyclo | ASP | PHE | ILE | Cyclo | TRP |
| | | | | | GLU | | | | LYS | | | | LYS | |
| | | | 39 | ASP | Cyclo | LYS | ALA | ALA | AIB | ASP | PHE | ILE | Cyclo | TRP |
| | | | | | LYS | | | | | | | | GLU | |
| | | | 40 | ASP | Cyclo | LYS | ALA | ALA | Cyclo | ASP | PHE | ILE | Cyclo | TRP |
| | | | | | GLU | | | | LYS | | | | LYS | |
| | | | 41 | ASP | Cyclo | LYS | ALA | ALA | AIB | ASP | PHE | ILE | Cyclo | TRP |
| | | | | | LYS | | | | | | | | GLU | |
| | | | 42 | ASP | Cyclo | LYS | ALA | ALA | AIB | ASP | PHE | ILE | Cyclo | TRP |
| | | | | | ASP | | | | | | | | GLU | |
| | | | 43 | ASP | Cyclo | LYS | ALA | ALA | Cyclo | ASP | PHE | ILE | Cyclo | TRP |
| | | | | | GLU | | | | LYS | | | | LYS | |
| | | | 44 | ASP | Cyclo | LYS | ALA | ALA | Cyclo | ASP | PHE | ILE | Cyclo | TRP |
| | | | | | GLU | | | | LYS | | | | GLU | |
| | | | 45 | ASP | Cyclo | LYS | ALA | ALA | Cyclo | ASP | PHE | ILE | Cyclo | TRP |
| | | | | | GLU | | | | LYS | | | | GLU | |
| | | | 46 | ASP | Cyclo | LYS | ALA | ALA | Cyclo | ASP | PHE | ILE | Cyclo | TRP |
| | | | | | GLU | | | | LYS | | | | GLU | |
| | | | 47 | ASP | Cyclo | LYS | ALA | ALA | AIB | ASP | PHE | ILE | Cyclo | TRP |
| | | | | | ASP | | | | | | | | GLU | |
| | | | 48 | ASP | Cyclo | Cyclo | ALA | ALA | Cyclo | ASP | PHE | ILE | Cyclo | TRP |
| | | | | | LYS | GLU | | | LYS | | | | GLU | |
| | | | 49 | ASP | Cyclo | Cyclo | ALA | ALA | LYS | Cyclo | PHE | ILE | Cyclo | TRP |
| | | | | | LYS | LYS | | | | ASP | | | GLU | |
| | | | 50 | ASP | Cyclo | LYS | ALA | ALA | Cyclo | ASP | PHE | ILE | Cyclo | TRP |
| | | | | | GLU | | | | LYS | | | | GLU | |
| | | | 51 | ASP | Cyclo | LYS | ALA | ALA | Cyclo | ASP | PHE | ILE | Cyclo | TRP |
| | | | | | GLU | | | | LYS | | | | GLU | |
| | | | 52 | ASP | Cyclo | Cyclo | ALA | ALA | Cyclo | ASP | PHE | ILE | Cyclo | TRP |
| | | | | | LYS | GLU | | | LYS | | | | GLU | |
| | | | 53 | ASP | Cyclo | Cyclo | ALA | ALA | LYS | Cyclo | PHE | ILE | Cyclo | TRP |
| | | | | | LYS | LYS | | | | ASP | | | GLU | |
| | | | 54 | ASP | Cyclo | LYS | ALA | ALA | Cyclo | ASP | PHE | ILE | Cyclo | TRP |
| | | | | | GLU | | | | LYS | | | | LYS | |
| | | | 55 | ASP | Cyclo | LYS | ALA | ALA | Cyclo | ASP | PHE | ILE | Cyclo | TRP |
| | | | | | GLU | | | | LYS | | | | LYS | |
| | | | 56 | ASP | Cyclo | LYS | ALA | ALA | Cyclo | ASP | PHE | ILE | Cyclo | TRP |
| | | | | | GLU | | | | LYS | | | | LYS | |
| | | | 57 | ASP | Cyclo | LYS- | ALA | ALA | Cyclo | ASP | PHE | ILE | Cyclo | TRP |
| | | | | | GLU | yGlu- | | | LYS | | | | LYS | |
| | | | | | | 2xOEG | | | | | | | | |
| | | | | | | C18 | | | | | | | | |
| | | | | | | diacid | | | | | | | | |
| | | | 58 | ASP | Cyclo | LYS- | ALA | ALA | Cyclo | ASP | PHE | ILE | Cyclo | TRP |
| | | | | | GLU | yGlu- | | | LYS | | | | LYS | |
| | | | | | | 2xOEG | | | | | | | | |
| | | | | | | C18 | | | | | | | | |
| | | | | | | diacid | | | | | | | | |
| | | | 59 | ASP | Cyclo | LYS | ALA | ALA | Cyclo | ASP | PHE | ILE | Cyclo | TRP |
| | | | | | GLU | | | | LYS | | | | LYS | |
| | | | 60 | ASP | Cyclo | LYS | ALA | ALA | Cyclo | ASP | PHE | ILE | Cyclo | TRP |
| | | | | | GLU | | | | LYS | | | | LYS | |
| | | | 61 | ASP | Cyclo | LYS- | ALA | ALA | Cyclo | ASP | PHE | ILE | Cyclo | TRP |
| | | | | | GLU | yGlu- | | | LYS | | | | LYS | |
| | | | | | | 2xOEG | | | | | | | | |
| | | | | | | C18 | | | | | | | | |
| | | | | | | diacid | | | | | | | | |
| | | | 62 | ASP | Cyclo | LYS- | ALA | ALA | Cyclo | ASP | PHE | ILE | Cyclo | TRP |
| | | | | | GLU | yGlu- | | | LYS | | | | LYS | |
| | | | | | | 2xOEG | | | | | | | | |
| | | | | | | C18 | | | | | | | | |
| | | | | | | diacid | | | | | | | | |
| | | | 63 | ASP | Cyclo | LYS | ALA | ALA | Cyclo | ASP | PHE | ILE | Cyclo | TRP |
| | | | | | GLU | | | | LYS | | | | GLU | |
| | | | 64 | ASP | Cyclo | LYS | ALA | ALA | Cyclo | ASP | PHE | ILE | Cyclo | TRP |
| | | | | | GLU | | | | LYS | | | | GLU | |
| | | | 65 | ASP | Cyclo | LYS | ALA | ALA | Cyclo | ASP | PHE | ILE | Cyclo | TRP |
| | | | | | GLU | | | | LYS | | | | GLU | |
| | | | 66 | ASP | Cyclo | LYS- | ALA | ALA | Cyclo | ASP | PHE | ILE | Cyclo | TRP |
| | | | | | GLU | yGlu- | | | LYS | | | | GLU | |
| | | | | | | 2xOEG | | | | | | | | |
| | | | | | | C18 | | | | | | | | |
| | | | | | | diacid | | | | | | | | |
| | | | 67 | ASP | Cyclo | LYS- | ALA | ALA | Cyclo | ASP | PHE | ILE | Cyclo | TRP |
| | | | | | GLU | yGlu- | | | LYS | | | | GLU | |
| | | | | | | 2xOEG | | | | | | | | |
| | | | | | | C18 | | | | | | | | |
| | | | | | | diacid | | | | | | | | |
| | | | 68 | ASP | Cyclo | LYS | ALA | ALA | Cyclo | ASP | PHE | ILE | Cyclo | TRP |
| | | | | | GLU | | | | LYS | | | | GLU | |
| | | | 69 | ASP | Cyclo | LYS | ALA | ALA | Cyclo | ASP | PHE | ILE | Cyclo | TRP |
| | | | | | GLU | | | | LYS | | | | GLU | |
| | | | 70 | ASP | Cyclo | LYS | ALA | ALA | Cyclo | ASP | PHE | ILE | Cyclo | TRP |
| | | | | | GLU | | | | LYS | | | | GLU | |
| | | | 71 | ASP | Cyclo | LYS | ALA | ALA | Cyclo | ASP | PHE | ILE | Cyclo | TRP |
| | | | | | GLU | | | | LYS | | | | GLU | |
| | | | 72 | ASP | Cyclo | LYS- | ALA | ALA | Cyclo | ASP | PHE | ILE | Cyclo | TRP |
| | | | | | GLU | yGlu- | | | LYS | | | | GLU | |
| | | | | | | 2xOEG | | | | | | | | |
| | | | | | | C18 | | | | | | | | |
| | | | | | | diacid | | | | | | | | |
| | | | 73 | ASP | Cyclo | LYS- | ALA | ALA | Cyclo | ASP | PHE | ILE | Cyclo | TRP |
| | | | | | GLU | yGlu- | | | LYS | | | | GLU | |
| | | | | | | 2xOEG | | | | | | | | |
| | | | | | | C18 | | | | | | | | |
| | | | | | | diacid | | | | | | | | |
| | | | 74 | ASP | Cyclo | Cyclo | ALA | ALA | Cyclo | ASP | PHE | ILE | Cyclo | TRP |
| | | | | | LYS | GLU | | | LYS | | | | GLU | |
| | | | 75 | ASP | Cyclo | Cyclo | ALA | ALA | LYS | Cyclo | PHE | ILE | Cyclo | TRP |
| | | | | | LYS | LYS | | | | ASP | | | GLU | |
| | | | 76 | ASP | Cyclo | Cyclo | ALA | ALA | Cyclo | ASP | PHE | ILE | Cyclo | TRP |
| | | | | | LYS | GLU | | | LYS | | | | GLU | |
| | | | 77 | ASP | Cyclo | Cyclo | ALA | ALA | LYS | Cyclo | PHE | ILE | Cyclo | TRP |
| | | | | | LYS | LYS | | | | ASP | | | GLU | |
| | | | 78 | ASP | Cyclo | Cyclo | ALA | ALA | Cyclo | ASP | PHE | ILE | Cyclo | TRP |
| | | | | | LYS | GLU | | | LYS | | | | GLU | |
| | | | 79 | ASP | Cyclo | Cyclo | ALA | ALA | LYS | Cyclo | PHE | ILE | Cyclo | TRP |
| | | | | | LYS | LYS | | | | ASP | | | GLU | |
| | | | 80 | ASP | Cyclo | Cyclo | ALA | ALA | Cyclo | ASP | PHE | ILE | Cyclo | TRP |
| | | | | | LYS | GLU | | | LYS | | | | GLU | |
| | | | 81 | ASP | Cyclo | Cyclo | ALA | ALA | LYS | Cyclo | PHE | ILE | Cyclo | TRP |
| | | | | | LYS | LYS | | | | ASP | | | GLU | |
| | | | | | | 26 | 27 | 28 | 29 | 30 | 31 | 32 | 33 | 34 |
| | | | | | 1 | LEU | ILE | GLN | THR | Cyclo | ILE | THR | Cyclo | — |
| | | | | | | | | | | GLU | | | LYS | |
| | | | | | 2 | LEU | ILE | GLN | THR | Cyclo | ILE | THR | Cyclo | — |
| | | | | | | | | | | GLU | | | LYS | |
| | | | | | 3 | LEU | ILE | Cyclo | THR | LYS | — | — | — | — |
| | | | | | | | | LYS | | | | | | |
| | | | | | 4 | LEU | ILE | GLN | Cyclo | LYS | ILE | THR | Cyclo | — |
| | | | | | | | | | GLU | | | | LYS | |
| | | | | | 5 | LEU | ILE | Cyclo | THR | LYS | — | — | — | — |
| | | | | | | | | LYS | | | | | | |
| | | | | | 6 | LEU | ILE | GLN | Cyclo | LYS | ILE | THR | Cyclo | — |
| | | | | | | | | | GLU | | | | LYS | |
| | | | | | 7 | LEU | ILE | ACPC | Cyclo | LYS | ILE | THR | Cyclo | — |
| | | | | | | | | | GLU | | | | LYS | |
| | | | | | 8 | LEU | ILE | GLN | THR | Cyclo | ILE | THR | Cyclo | — |
| | | | | | | | | | | GLU | | | LYS | |
| | | | | | 9 | LEU | ILE | GLN | THR | Cyclo | ILE | THR | Cyclo | — |
| | | | | | | | | | | GLU | | | LYS | |
| | | | | | 10 | LEU | ILE | GLN | Cyclo | LYS | ILE | THR | Cyclo | — |
| | | | | | | | | | GLU | | | | LYS | |
| | | | | | 11 | LEU | ILE | GLN | THR | Cyclo | ILE | THR | Cyclo | — |
| | | | | | | | | | | GLU | | | LYS | |
| | | | | | 12 | LEU | ILE | GLN | THR | Cyclo | ILE | THR | Cyclo | — |
| | | | | | | | | | | GLU | | | LYS | |
| | | | | | 13 | LEU | ILE | GLN | THR | Cyclo | ILE | THR | Cyclo | PRO |
| | | | | | | | | | | GLU | | | LYS | |
| | | | | | 14 | LEU | ILE | GLN | THR | Cyclo | α- | THR | Cyclo | — |
| | | | | | | | | | | GLU | MeLEU | | LYS | |
| | | | | | 15 | LEU | ILE | GLN | THR | Cyclo | ILE | THR | Cyclo | — |
| | | | | | | | | | | GLU | | | LYS | |
| | | | | | 16 | LEU | ILE | GLN | THR | Cyclo | ILE | THR | Cyclo | — |
| | | | | | | | | | | GLU | | | LYS | |
| | | | | | 17 | LEU | ILE | Cyclo | THR | LYS | — | — | — | — |
| | | | | | | | | LYS | | | | | | |
| | | | | | 18 | LEU | ILE | GLN | Cyclo | LYS | ILE | THR | Cyclo | — |
| | | | | | | | | | GLU | | | | LYS | |
| | | | | | 19 | LEU | ILE | GLN | Cyclo | LYS | ILE | THR | Cyclo | — |
| | | | | | | | | | GLU | | | | LYS | |
| | | | | | 20 | LEU | ILE | GLN | Cyclo | LYS | ILE | THR | Cyclo | — |
| | | | | | | | | | GLU | | | | LYS | |
| | | | | | 21 | LEU | ILE | Cyclo | THR | LYS | — | — | — | — |
| | | | | | | | | LYS | | | | | | |
| | | | | | 22 | LEU | ILE | GLN | Cyclo | LYS | ILE | THR | Cyclo | — |
| | | | | | | | | | GLU | | | | LYS | |
| | | | | | 23 | LEU | ILE | GLN | THR | Cyclo | ILE | THR | Cyclo | — |
| | | | | | | | | | | GLU | | | LYS | |
| | | | | | 24 | LEU | ILE | GLN | Cyclo | LYS | ILE | THR | Cyclo | — |
| | | | | | | | | | GLU | | | | LYS | |
| | | | | | 25 | LEU | ILE | GLN | Cyclo | LYS | ILE | THR | Cyclo | — |
| | | | | | | | | | GLU | | | | LYS | |
| | | | | | 26 | LEU | ILE | GLN | Cyclo | LYS | ILE | THR | Cyclo | — |
| | | | | | | | | | GLU | | | | LYS | |
| | | | | | 27 | LEU | ILE | Cyclo | THR | LYS | — | — | — | — |
| | | | | | | | | LYS | | | | | | |
| | | | | | 28 | LEU | ILE | Cyclo | THR | LYS | — | — | — | — |
| | | | | | | | | LYS | | | | | | |
| | | | | | 29 | LEU | ILE | Cyclo | THR | PRO | — | — | — | — |
| | | | | | | | | LYS | | | | | | |
| | | | | | 30 | LEU | ILE | Cyclo | THR | LYS | PRO | — | — | — |
| | | | | | | | | LYS | | | | | | |
| | | | | | 31 | LEU | ILE | Cyclo | THR | LYS | — | — | — | — |
| | | | | | | | | LYS | | | | | | |
| | | | | | 32 | LEU | ILE | Cyclo | THR | LYS | — | — | — | — |
| | | | | | | | | LYS | | | | | | |
| | | | | | 33 | LEU | ILE | Cyclo | THR | LYS | — | — | — | — |
| | | | | | | | | LYS | | | | | | |
| | | | | | 34 | LEU | ILE | Cyclo | THR | LYS | — | — | — | — |
| | | | | | | | | LYS | | | | | | |
| | | | | | 35 | LEU | ILE | Cyclo | THR | LYS | — | — | — | — |
| | | | | | | | | LYS | | | | | | |
| | | | | | 36 | LEU | ILE | Cyclo | THR | LYS | — | — | — | — |
| | | | | | | | | LYS | | | | | | |
| | | | | | 37 | LEU | ILE | Cyclo | THR | PIPALA | — | — | — | — |
| | | | | | | | | LYS | | | | | | |
| | | | | | 38 | LEU | ILE | Cyclo | THR | Cyclo | ILE | THR | Cyclo | — |
| | | | | | | | | ASP | | GLU | | | LYS | |
| | | | | | 39 | LEU | ILE | Cyclo | Cyclo | LYS | ILE | THR | Cyclo | — |
| | | | | | | | | LYS | GLU | | | | LYS | |
| | | | | | 40 | LEU | ILE | Cyclo | Cyclo | LYS | ILE | THR | Cyclo | — |
| | | | | | | | | ASP | GLU | | | | LYS | |
| | | | | | 41 | LEU | ILE | Cyclo | Cyclo | LYS | ILE | THR | Cyclo | — |
| | | | | | | | | LYS | GLU | | | | LYS | |
| | | | | | 42 | LEU | ILE | Cyclo | Cyclo | LYS | ILE | THR | Cyclo | — |
| | | | | | | | | LYS | GLU | | | | LYS | |
| | | | | | 43 | LEU | ILE | Cyclo | Cyclo | LYS | ILE | THR | Cyclo | — |
| | | | | | | | | ASP | GLU | | | | LYS | |
| | | | | | 44 | LEU | ILE | Cyclo | THR | Cyclo | ILE | THR | Cyclo | — |
| | | | | | | | | LYS | | GLU | | | LYS | |
| | | | | | 45 | LEU | ILE | Cyclo | Cyclo | LYS | ILE | THR | Cyclo | — |
| | | | | | | | | LYS | GLU | | | | LYS | |
| | | | | | 46 | LEU | ILE | Cyclo | Cyclo | LYS | ILE | THR | Cyclo | — |
| | | | | | | | | LYS | GLU | | | | LYS | |
| | | | | | 47 | LEU | ILE | Cyclo | Cyclo | LYS | ILE | THR | Cyclo | — |
| | | | | | | | | LYS | GLU | | | | LYS | |
| | | | | | 48 | LEU | ILE | Cyclo | Cyclo | LYS | ILE | THR | Cyclo | — |
| | | | | | | | | LYS | LYS | | | | GLU | |
| | | | | | 49 | LEU | ILE | Cyclo | Cyclo | LYS | ILE | THR | Cyclo | — |
| | | | | | | | | LYS | LYS | | | | GLU | |
| | | | | | 50 | LEU | ILE | Cyclo | Cyclo | LYS | ILE | THR | Cyclo | — |
| | | | | | | | | LYS | GLU | | | | LYS | |
| | | | | | 51 | LEU | ILE | Cyclo | Cyclo | LYS | ILE | THR | Cyclo | — |
| | | | | | | | | LYS | GLU | | | | LYS | |
| | | | | | 52 | LEU | ILE | Cyclo | Cyclo | LYS | ILE | THR | Cyclo | — |
| | | | | | | | | LYS | LYS | | | | GLU | |
| | | | | | 53 | LEU | ILE | Cyclo | Cyclo | LYS | ILE | THR | Cyclo | — |
| | | | | | | | | LYS | LYS | | | | GLU | |
| | | | | | 54 | LEU | ILE | Cyclo | Cyclo | LYS | ILE | THR | Cyclo | — |
| | | | | | | | | ASP | GLU | | | | LYS | |
| | | | | | 55 | LEU | ILE | Cyclo | Cyclo | LYS | ILE | THR | Cyclo | — |
| | | | | | | | | ASP | GLU | | | | LYS | |
| | | | | | 56 | LEU | ILE | Cyclo | Cyclo | LYS | ILE | THR | Cyclo | — |
| | | | | | | | | ASP | GLU | | | | LYS | |
| | | | | | 57 | LEU | ILE | Cyclo | Cyclo | LYS | ILE | THR | Cyclo | — |
| | | | | | | | | ASP | GLU | | | | LYS | |
| | | | | | 58 | LEU | ILE | Cyclo | Cyclo | LYS | ILE | THR | Cyclo | — |
| | | | | | | | | ASP | GLU | | | | LYS | |
| | | | | | 59 | LEU | ILE | Cyclo | Cyclo | LYS | ILE | THR | Cyclo | — |
| | | | | | | | | ASP | GLU | | | | LYS | |
| | | | | | 60 | LEU | ILE | Cyclo | Cyclo | LYS | ILE | THR | Cyclo | — |
| | | | | | | | | ASP | GLU | | | | LYS | |
| | | | | | 61 | LEU | ILE | Cyclo | Cyclo | LYS | ILE | THR | Cyclo | — |
| | | | | | | | | ASP | GLU | | | | LYS | |
| | | | | | 62 | LEU | ILE | Cyclo | Cyclo | LYS | ILE | THR | Cyclo | — |
| | | | | | | | | ASP | GLU | | | | LYS | |
| | | | | | 63 | LEU | ILE | Cyclo | Cyclo | LYS | ILE | THR | Cyclo | — |
| | | | | | | | | LYS | GLU | | | | LYS | |
| | | | | | 64 | LEU | ILE | Cyclo | Cyclo | LYS | ILE | THR | Cyclo | — |
| | | | | | | | | LYS | GLU | | | | LYS | |
| | | | | | 65 | LEU | ILE | Cyclo | Cyclo | LYS | ILE | THR | Cyclo | — |
| | | | | | | | | LYS | GLU | | | | LYS | |
| | | | | | 66 | LEU | ILE | Cyclo | Cyclo | LYS | ILE | THR | Cyclo | — |
| | | | | | | | | LYS | GLU | | | | LYS | |
| | | | | | 67 | LEU | ILE | Cyclo | Cyclo | LYS | ILE | THR | Cyclo | — |
| | | | | | | | | LYS | GLU | | | | LYS | |
| | | | | | 68 | LEU | ILE | Cyclo | Cyclo | LYS | ILE | THR | Cyclo | — |
| | | | | | | | | LYS | GLU | | | | LYS | |
| | | | | | 69 | LEU | ILE | Cyclo | Cyclo | LYS | ILE | THR | Cyclo | — |
| | | | | | | | | LYS | GLU | | | | LYS | |
| | | | | | 70 | LEU | ILE | Cyclo | Cyclo | LYS | ILE | THR | Cyclo | — |
| | | | | | | | | LYS | GLU | | | | LYS | |
| | | | | | 71 | LEU | ILE | Cyclo | Cyclo | LYS | ILE | THR | Cyclo | — |
| | | | | | | | | LYS | GLU | | | | LYS | |
| | | | | | 72 | LEU | ILE | Cyclo | Cyclo | LYS | ILE | THR | Cyclo | — |
| | | | | | | | | LYS | GLU | | | | LYS | |
| | | | | | 73 | LEU | ILE | Cyclo | Cyclo | LYS | ILE | THR | Cyclo | — |
| | | | | | | | | LYS | GLU | | | | LYS | |
| | | | | | 74 | LEU | ILE | Cyclo | Cyclo | LYS | ILE | THR | Cyclo | — |
| | | | | | | | | LYS | LYS | | | | GLU | |
| | | | | | 75 | LEU | ILE | Cyclo | Cyclo | LYS | ILE | THR | Cyclo | — |
| | | | | | | | | LYS | LYS | | | | GLU | |
| | | | | | 76 | LEU | ILE | Cyclo | Cyclo | LY8 | ILE | THR | Cyclo | — |
| | | | | | | | | LYS | LYS | | | | GLU | |
| | | | | | 77 | LEU | ILE | Cyclo | Cyclo | LYS | ILE | THR | Cyclo | — |
| | | | | | | | | LYS | LYS | | | | GLU | |
| | | | | | 78 | LEU | ILE | Cyclo | Cyclo | LYS | ILE | THR | Cyclo | — |
| | | | | | | | | LYS | LYS | | | | GLU | |
| | | | | | 79 | LEU | ILE | Cyclo | Cyclo | LYS | ILE | THR | Cyclo | — |
| | | | | | | | | LYS | LYS | | | | GLU | |
| | | | | | 80 | LEU | ILE | Cyclo | Cyclo | LYS | ILE | THR | Cyclo | — |
| | | | | | | | | LYS | LYS | | | | GLU | |
| | | | | | 81 | LEU | ILE | Cyclo | Cyclo | LYS | ILE | THR | Cyclo | — |
| | | | | | | | | LYS | LYS | | | | GLU | |

TABLE 1a
Examples 82-117

| Ex | 1 | 2 | 3 | 4 | 5 | 6 | 7 | 8 | 9 | 10 | 11 | 12 | 13 | 14 | 15 | 16 | 17 |
|---|---|---|---|---|---|---|---|---|---|---|---|---|---|---|---|---|---|
| 82 | — | CAP | TET | GLY | SER | PHE | SER | Cyclo | GLU | NLE | D- | Cyclo | ILE | LEU | ASP | LEU | LYS |
| | | | | | | | | LYS | | | PHE | ASP | | | | | |
| 83 | — | CAP | TET | GLY | SER | PHE | SER | ASP | GLU | NLE | D- | THR | ILE | LEU | ASP | LEU | LYS |
| | | | | | | | | | | | PHE | | | | | | |
| 84 | HIS | AIB | ASP | Cyclo | SER | PHE | SER | Cyclo | GLU | NLE | D- | THR | ILE | LEU | ASP | LEU | LYS |
| | | | | LYS | | | | ASP | | | PHE | | | | | | |
| 85 | HIS | AIB | ASP | GLY | SER | PHE | SER | Cyclo | GLU | NLE | D- | Cyclo | ILE | LEU | ASP | LEU | LYS |
| | | | | | | | | LYS | | | PHE | ASP | | | | | |
| 86 | HIS | AIB | ASP | GLY | SER | PHE | SER | ASP | GLU | NLE | Cyclo | THR | ILE | LEU | Cyclo | LEU | LYS |
| | | | | | | | | | | | ASP | | | | LYS | | |
| 87 | HIS | AIB | ASP | GLY | SER | PHE | SER | ASP | GLU | NLE | D- | Cyclo | ILE | LEU | ASP | Cyclo | LYS |
| | | | | | | | | | | | PHE | GLU | | | | LYS | |
| 88 | HIS | AIB | ASP | GLY | SER | PHE | SER | ASP | GLU | NLE | D- | Cyclo | ILE | LEU | ASP | Cyclo | LYS |
| | | | | | | | | | | | PHE | LYS | | | | ASP | |
| 89 | HIS | AIB | ASP | GLY | SER | PHE | SER | ASP | GLU | NLE | D- | THR | ILE | LEU | ASP | Cyclo | LYS |
| | | | | | | | | | | | PHE | | | | | GLU | |
| 90 | HIS | AIB | ASP | GLY | SER | PHE | SER | ASP | GLU | NLE | D- | THR | ILE | LEU | ASP | LEU | LYS |
| | | | | | | | | | | | PHE | | | | | | |
| 91 | — | CAP | TET | GLY | SER | PHE | SER | ASP | GLU | NLE | D- | Cyclo | ILE | LEU | ASP | Cyclo | LYS |
| | | | | | | | | | | | PHE | LYS | | | | ASP | |
| 92 | HIS | AIB | ASP | GLY | SER | PHE | SER | ASP | GLU | NLE | D- | THR | ILE | LEU | ASP | Cyclo | LYS |
| | | | | | | | | | | | PHE | | | | | ASP | |
| 93 | HIS | AIB | ASP | GLY | SER | PHE | SER | ASP | GLU | NLE | D- | THR | ILE | LEU | ASP | LEU | LYS |
| | | | | | | | | | | | PHE | | | | | | |
| 94 | HIS | AIB | ASP | GLY | SER | PHE | SER | ASP | GLU | NLE | Cyclo | THR | ILE | LEU | ASP | LEU | LYS |
| | | | | | | | | | | | LYS | | | | | | |
| 95 | — | CAP | TET | GLY | SER | PHE | SER | ASP | GLU | NLE | D- | THR | ILE | LEU | ASP | Cyclo | LYS |
| | | | | | | | | | | | PHE | | | | | ASP | |
| 96 | — | CAP | TET | GLY | SER | PHE | SER | ASP | GLU | NLE | D- | THR | ILE | LEU | ASP | Cyclo | LYS |
| | | | | | | | | | | | PHE | | | | | GLU | |
| 97 | HIS | AIB | ASP | GLY | SER | PHE | SER | ASP | GLU | NLE | D- | Cyclo | ILE | LEU | ASP | Cyclo | LYS |
| | | | | | | | | | | | PHE | ASP | | | | LYS | |
| 98 | HIS | AIB | ASP | GLY | SER | PHE | SER | ASP | GLU | NLE | D- | THR | ILE | LEU | ASP | LEU | Cyclo |
| | | | | | | | | | | | PHE | | | | | | GLU |
| 99 | HIS | AIB | ASP | GLY | SER | PHE | SER | ASP | GLU | NLE | D- | THR | ILE | LEU | ASP | LEU | LYS |
| | | | | | | | | | | | PHE | | | | | | |
| 100 | HIS | AIB | ASP | GLY | SER | PHE | SER | ASP | GLU | NLE | D- | THR | ILE | LEU | ASP | LEU | LYS |
| | | | | | | | | | | | PHE | | | | | | |
| 101 | HIS | AIB | ASP | GLY | SER | PHE | SER | ASP | GLU | NLE | D- | THR | ILE | LEU | ASP | LEL | LYS |
| | | | | | | | | | | | PHE | | | | | | |
| 102 | HIS | AIB | ASP | GLY | SER | PHE | SER | ASP | GLU | NLE | D- | THR | ILE | LEU | ASP | Cyclo | LYS |
| | | | | | | | | | | | PHE | | | | | LYS | |
| 103 | HIS | AIB | ASP | GLY | SER | PHE | SER | ASP | GLU | NLE | D- | THR | ILE | LEU | ASP | Cyclo | LYS |
| | | | | | | | | | | | PHE | | | | | LYS | |
| 104 | HIS | AIB | ASP | Cyclo | SER | PHE | SER | Cyclo | GLU | NLE | D- | THR | ILE | LEU | ASP | LEU | LYS |
| | | | | GLU | | | | LYS | | | PHE | | | | | | |
| 105 | — | CAP | TET | GLY | SER | PHE | SER | ASP | GLU | NLE | D- | Cyclo | ILE | LEU | ASP | Cyclo | LYS |
| | | | | | | | | | | | PHE | LYS | | | | ASP | |
| 106 | — | CAP | TET | GLY | SER | PHE | SER | ASP | GLU | NLE | D- | THR | ILE | LEU | ASP | Cyclo | LYS |
| | | | | | | | | | | | PHE | | | | | ASP | |
| 107 | — | CAP | TET | GLY | SER | PHE | SER | ASP | GLU | NLE | D- | THR | ILE | LEU | ASP | Cyclo | LYS |
| | | | | | | | | | | | PHE | | | | | GLU | |
| 108 | — | CAP | TET | GLY | SER | PHE | SER | Cyclo | GLU | NLE | D- | Cyclo | ILE | LEU | ASP | LEU | LYS |
| | | | | | | | | LYS | | | PHE | ASP | | | | | |
| 109 | — | CAP | TET | GLY | SER | PHE | SER | ASP | GLU | NLE | D- | Cyclo | ILE | LEU | ASP | Cyclo | LYS |
| | | | | | | | | | | | PHE | LYS | | | | ASP | |
| 110 | HIS | AIB | ASP | Cyclo | SER | PHE | SER | Cyclo | GLU | NLE | D- | THR | ILE | LEU | ASP | Cyclo | LYS |
| | | | | GLU | | | | LYS | | | PHE | | | | | LYS | |
| 111 | HIS | AIB | ASP | GLY | SER | PHE | Cyclo | ASP | GLU | NLE | Cyclo | THR | ILE | LEU | ASP | LEU | LYS |
| | | | | | | | GLU | | | | LYS | | | | | | |
| 112 | HIS | AIB | ASP | GLY | SER | PHE | SER | ASP | GLU | NLE | Cyclo | THR | ILE | LEU | Cyclo | LEU | LYS |
| | | | | | | | | | | | ASP | | | | LYS | | |
| 113 | HIS | AIB | ASP | GLY | SER | PHE | SER | ASP | GLU | NLE | D- | THR | ILE | LEU | ASP | Cyclo | LYS |
| | | | | | | | | | | | PHE | | | | | LYS | |
| 114 | — | CAP | TET | GLY | SER | PHE | SER | Cyclo | GLU | NLE | D- | Cyclo | ILE | LEU | ASP | Cyclo | LYS |
| | | | | | | | | LYS | | | PHE | ASP | | | | ASP | |
| 115 | — | CAP | TET | GLY | SER | PHE | SER | Cyclo | GLU | NLE | D- | Cyclo | ILE | LEU | ASP | Cyclo | LYS |
| | | | | | | | | LYS | | | PHE | ASP | | | | GLU | |
| 116 | — | CAP | TET | D- | SER | PHE | SER | ASP | GLU | NLE | D- | THR | α- | LEU | ASP | Cyclo | LYS |
| | | | | ALA | | | | | | | PHE | | MeLEU | | | GLU | |
| 117 | — | CAP | TET | D- | SER | PHE | SER | ASP | GLU | NLE | D- | THR | α- | LEU | ASP | Cyclo | LYS |
| | | | | ALA | | | | | | | PHE | | MeLEU | | | GLU | |
| | | Examples 82-117 | | | | | | | | | | | | | | | |
| | Ex | 18 | 19 | 20 | 21 | 22 | 23 | 24 | 25 | 26 | 27 | 28 | 29 | 30 | 31 | 32 | 33 |
| | 82 | ALA | ALA | LYS | ASP | PHE | ILE | ASN | TRP | LEU | ILE | GLN | THR | LYS | ILE | THR | PHE |
| | 83 | ALA | ALA | LYS | ASP | PHE | ILE | ASN | TRP | LEU | ILE | GLN | THR | Cyclo | ILE | THR | Cyclo |
| | | | | | | | | | | | | | | GLU | | | LYS |
| | 84 | ALA | ALA | LYS | ASP | PHE | ILE | ASN | TRP | LEU | ILE | GLN | THR | LYS | ILE | THR | ASP |
| | 85 | ALA | ALA | LYS | ASP | PHE | ILE | ASN | TRP | LEU | ILE | GLN | THR | LYS | ILE | THR | ASP |
| | 86 | ALA | ALA | LYS | ASP | PHE | ILE | ASN | TRP | LEU | ILE | GLN | THR | LYS | ILE | THR | ASP |
| | 87 | ALA | ALA | LYS | ASP | PHE | ILE | ASN | TRP | LEU | ILE | GLN | THR | LYS | ILE | THR | ASP |
| | 88 | ALA | ALA | LYS | ASP | PHE | ILE | ASN | TRP | LEU | ILE | GLN | THR | LYS | ILE | THR | ASP |
| | 89 | ALA | ALA | Cyclo | ASP | PHE | ILE | ASN | TRP | LEU | ILE | GLN | THR | LYS | ILE | THR | ASP |
| | | | | LYS | | | | | | | | | | | | | |
| | 90 | ALA | ALA | LYS | ASP | PHE | ILE | ASN | TRP | LEU | ILE | GLN | THR | Cyclo | ILE | THR | Cyclo |
| | | | | | | | | | | | | | | GLU | | | LYS |
| | 91 | ALA | ALA | LYS | ASP | PHE | ILE | ASN | TRP | LEU | ILE | GLN | THR | LYS | ILE | THR | PHE |
| | 92 | ALA | ALA | Cyclo | ASP | PHE | ILE | ASN | TRP | LEU | ILE | GLN | THR | LYS | ILE | THR | ASP |
| | | | | LYS | | | | | | | | | | | | | |
| | 93 | ALA | ALA | LYS | ASP | PHE | ILE | ASN | TRP | LEU | ILE | GLN | Cyclo | LYS | ILE | THR | Cyclo |
| | | | | | | | | | | | | | LYS | | | | GLU |
| | 94 | ALA | ALA | LYS | ASP | PHE | ILE | ASN | TRP | LEU | ILE | GLN | THR | LYS | ILE | THR | ASP |
| | 95 | ALA | ALA | Cyclo | ASP | PHE | ILE | ASN | TRP | LEU | ILE | GLN | THR | LYS | ILE | THR | PHE |
| | | | | LYS | | | | | | | | | | | | | |
| | 96 | ALA | ALA | Cyclo | ASP | PHE | ILE | ASN | TRP | LEU | ILE | GLN | THR | LYS | ILE | THR | PHE |
| | | | | LYS | | | | | | | | | | | | | |
| | 97 | ALA | ALA | LYS | ASP | PHE | ILE | ASN | TRP | LEU | ILE | GLN | THR | LYS | ILE | THR | ASP |
| | 98 | ALA | ALA | Cyclo | ASP | PHE | ILE | ASN | TRP | LEU | ILE | GLN | THR | LYS | ILE | THR | ASP |
| | | | | LYS | | | | | | | | | | | | | |
| | 99 | ALA | ALA | LYS | ASP | PHE | ILE | Cyclo | TRP | LEU | ILE | Cyclo | THR | LYS | ILE | THR | ASP |
| | | | | | | | | GLU | | | | LYS | | | | | |
| | 100 | ALA | ALA | LYS | ASP | PHE | ILE | Cyclo | TRP | LEU | ILE | Cyclo | THR | LYS | ILE | THR | ASP |
| | | | | | | | | LYS | | | | ASP | | | | | |
| | 101 | ALA | ALA | LYS | ASP | PHE | ILE | ASN | TRP | LEU | ILE | GLN | THR | Cyclo | ILE | THR | Cyclo |
| | | | | | | | | | | | | | | LYS | | | GLU |
| | 102 | ALA | ALA | Cyclo | ASP | PHE | ILE | ASN | TRP | LEU | ILE | GLN | THR | LYS | ILE | THR | ASP |
| | | | | GLU | | | | | | | | | | | | | |
| | 103 | ALA | ALA | Cyclo | ASP | PHE | ILE | ASN | TRP | LEU | ILE | GLN | THR | LYS | ILE | THR | ASP |
| | | | | ASP | | | | | | | | | | | | | |
| | 104 | ALA | ALA | LYS | ASP | PHE | ILE | ASN | TRP | LEU | ILE | GLN | THR | Cyclo | ILE | THR | Cyclo |
| | | | | | | | | | | | | | | GLU | | | LYS |
| | 105 | ALA | ALA | LYS | ASP | PHE | ILE | ASN | TRP | LEU | ILE | GLN | THR | Cyclo | ILE | THR | Cyclo |
| | | | | | | | | | | | | | | GLU | | | LYS |
| | 106 | ALA | ALA | Cyclo | ASP | PHE | ILE | ASN | TRP | LEU | ILE | GLN | THR | Cyclo | ILE | THR | Cyclo |
| | | | | LYS | | | | | | | | | | GLU | | | LYS |
| | 107 | ALA | ALA | Cyclo | ASP | PHE | ILE | ASN | TRP | LEU | ILE | GLN | THR | Cyclo | ILE | THR | Cyclo |
| | | | | LYS | | | | | | | | | | GLU | | | LYS |
| | 108 | ALA | ALA | AIB | ASP | PHE | ILE | ASN | TRP | LEU | ILE | GLN | THR | Cyclo | ILE | THR | Cyclo |
| | | | | | | | | | | | | | | GLU | | | LYS |
| | 109 | ALA | ALA | AIB | ASP | PHE | ILE | ASN | TRP | LEU | ILE | GLN | THR | Cyclo | ILE | THR | Cyclo |
| | | | | | | | | | | | | | | GLU | | | LYS |
| | 110 | ALA | ALA | Cyclo | ASP | PHE | ILE | ASN | TRP | LEU | ILE | GLN | THR | LYS | ILE | THR | ASP |
| | | | | ASP | | | | | | | | | | | | | |
| | 111 | ALA | ALA | LYS | ASP | PHE | ILE | ASN | TRP | LEU | ILE | GLN | THR | Cylo | ILE | THR | Cyclo |
| | | | | | | | | | | | | | | GLU | | | LYS |
| | 112 | ALA | ALA | LYS | ASP | PHE | ILE | ASN | TRP | LEU | ILE | GLN | Cyclo | LYS | ILE | THR | Cyclo |
| | | | | | | | | | | | | | LYS | | | | GLU |
| | 113 | ALA | ALA | Cyclo | ASP | PHE | ILE | ASN | TRP | LEU | ILE | GLN | Cyclo | LYS | ILE | THR | Cyclo |
| | | | | GLU | | | | | | | | | LYS | | | | GLU |
| | 114 | ALA | ALA | Cyclo | ASP | PHE | ILE | ASN | TRP | LEU | ILE | GLN | THR | LYS | ILE | THR | PHE |
| | | | | LYS | | | | | | | | | | | | | |
| | 115 | ALA | ALA | Cyclo | ASP | PHE | ILE | ASN | TRP | LEU | ILE | GLN | THR | LYS | ILE | THR | PHE |
| | | | | LYS | | | | | | | | | | | | | |
| | 116 | ALA | ALA | Cyclo | ASP | PHE | ILE | Cyclo | TRP | LEU | ILE | Cyclo | THR | LYS | ILE | THR | PHE |
| | | | | LYS | | | | GLU | | | | LYS | | | | | |
| | 117 | ALA | ALA | Cyclo | ASP | PHE | ILE | Cyclo | TRP | LEU | ILE | Cyclo | THR | LYS | — | — | — |
| | | | | LYS | | | | GLU | | | | LYS | | | | | |

Standard amino acid symbols are used in Table 1 and Table 1a where appropriate. In cases where a standard symbol is not available, the following representations are used:

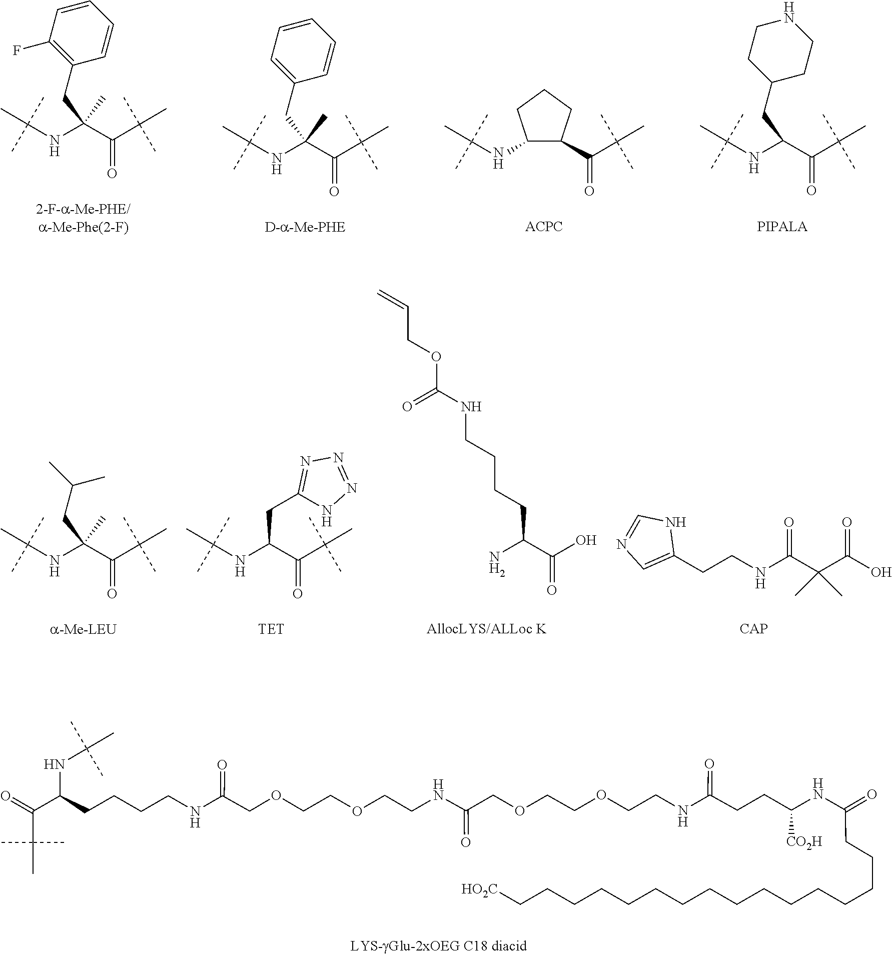

CycloLYS, for example refers to a lysine residue which is joined to another residue via a lactam bridge.

Exemplary structures of certain examples are shown below:

### Example 30

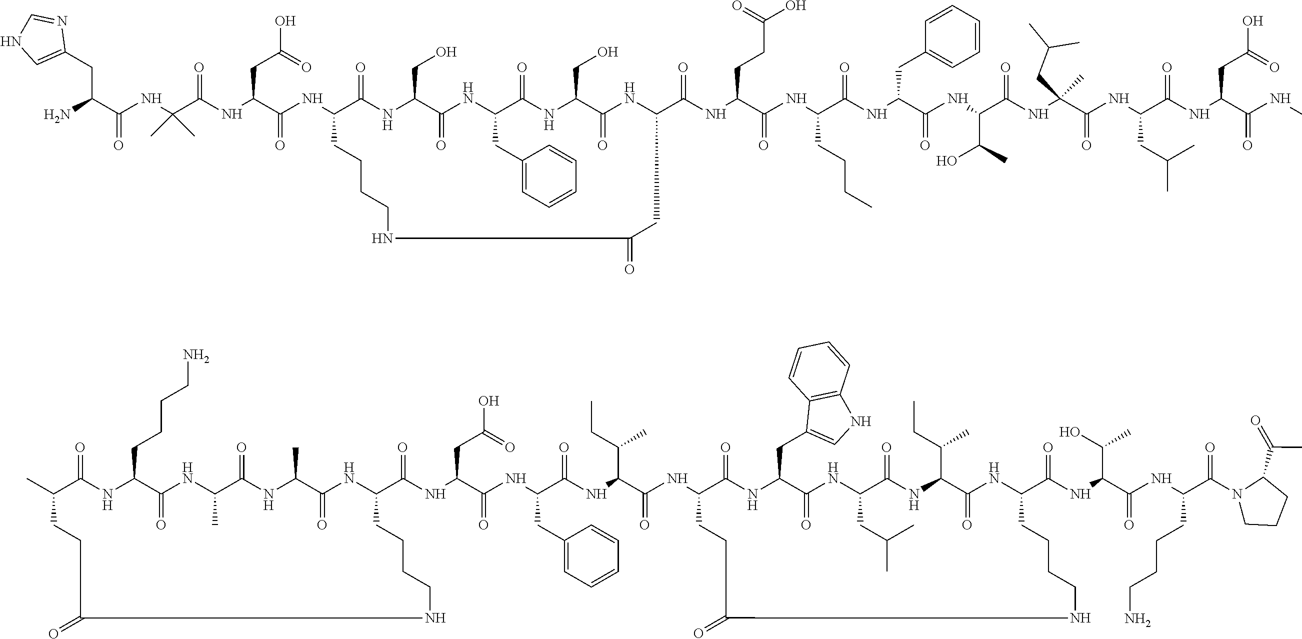

### Example 31

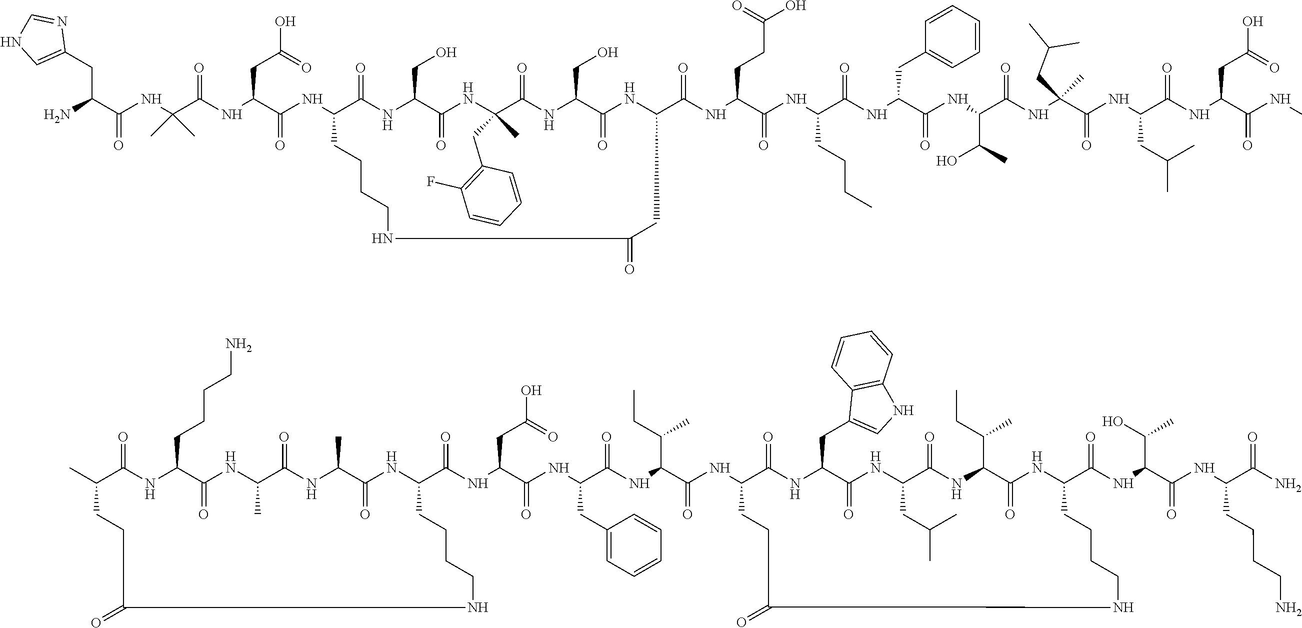

### Example 46

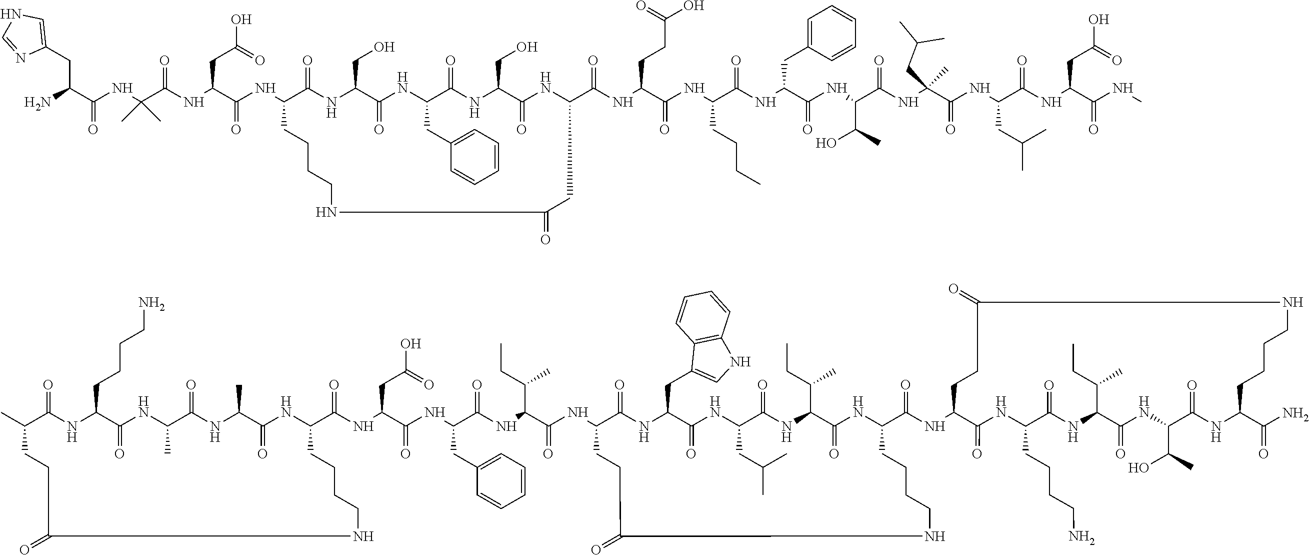

### Example 48

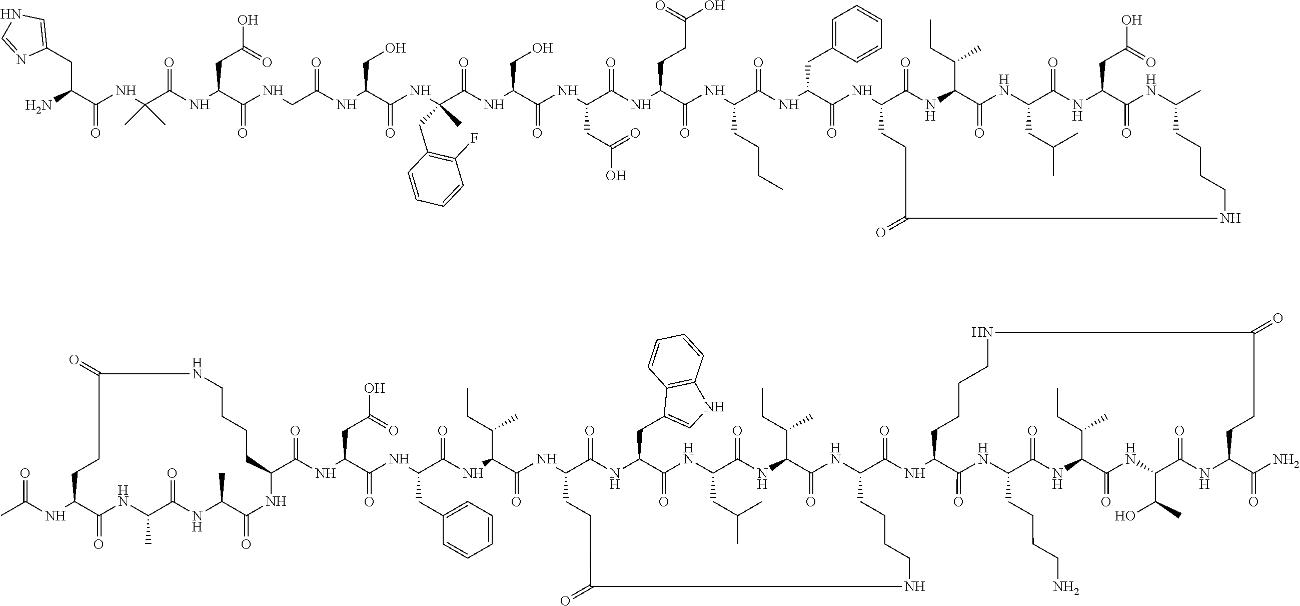

### Example 52

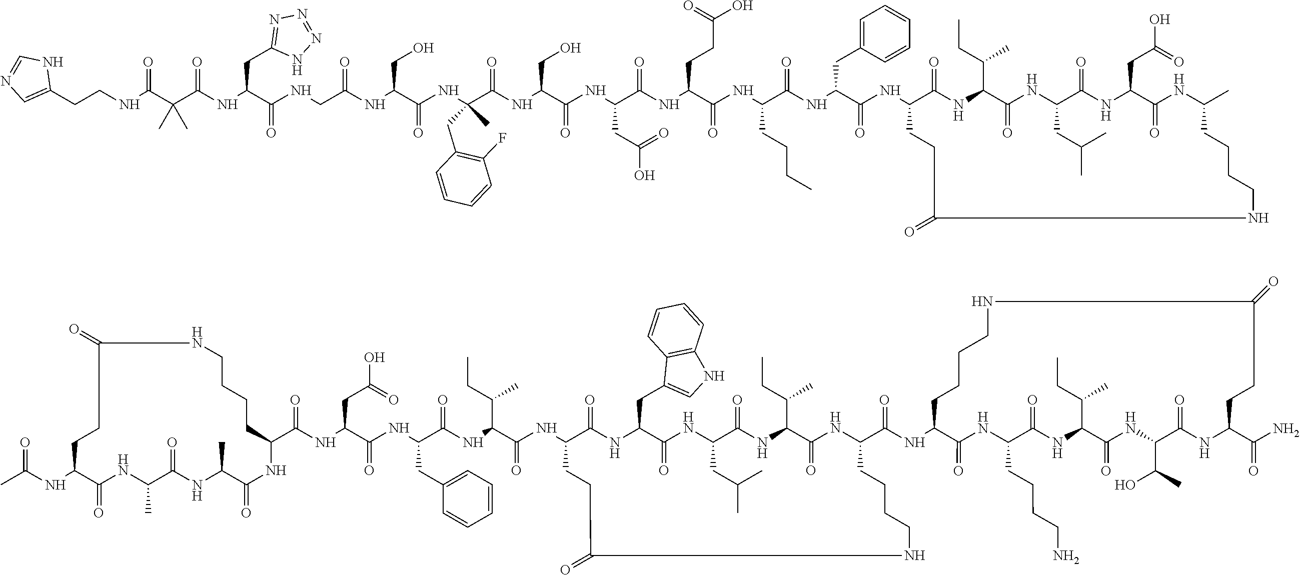

### Example 55

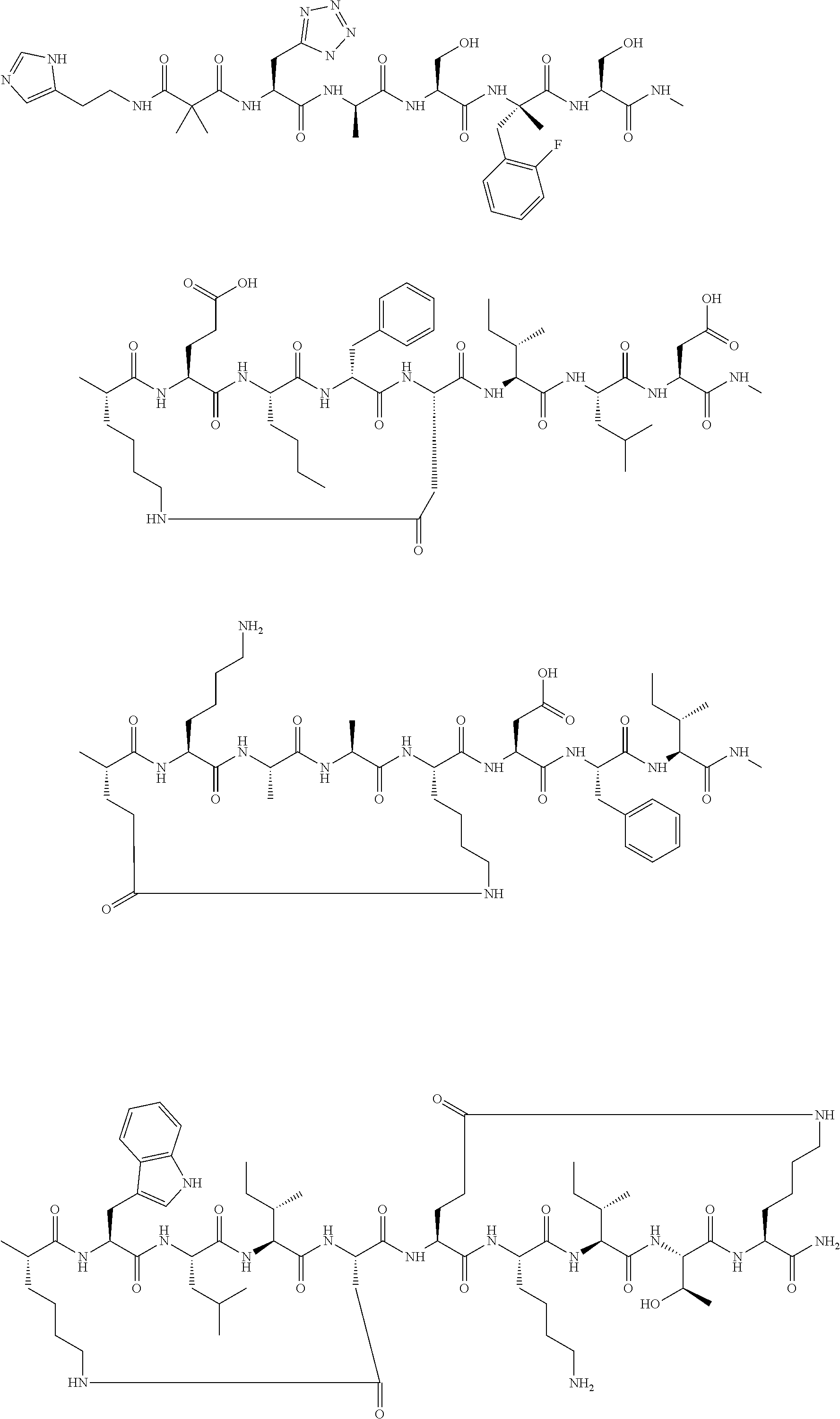

### Example 60

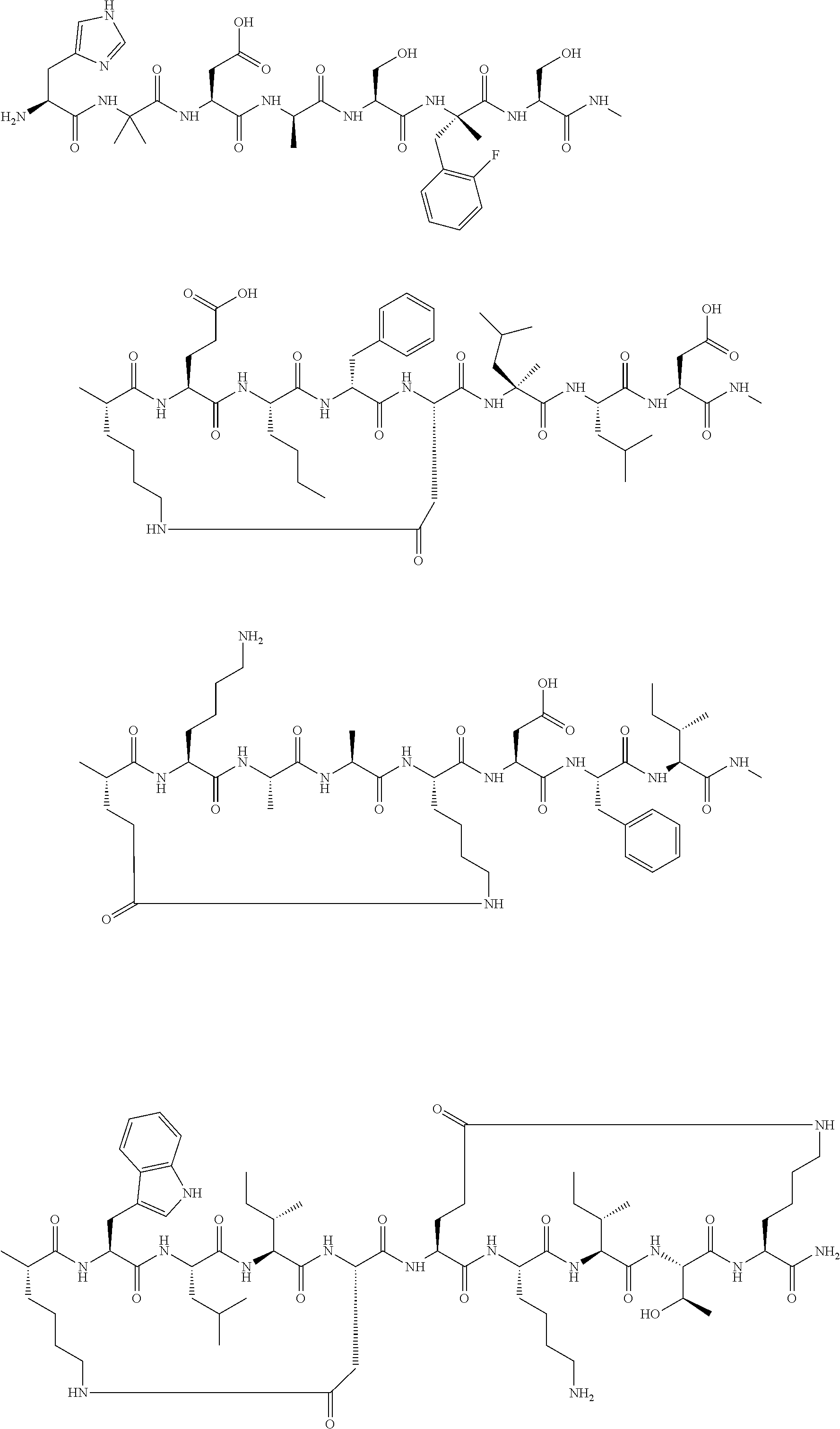

### Example 74

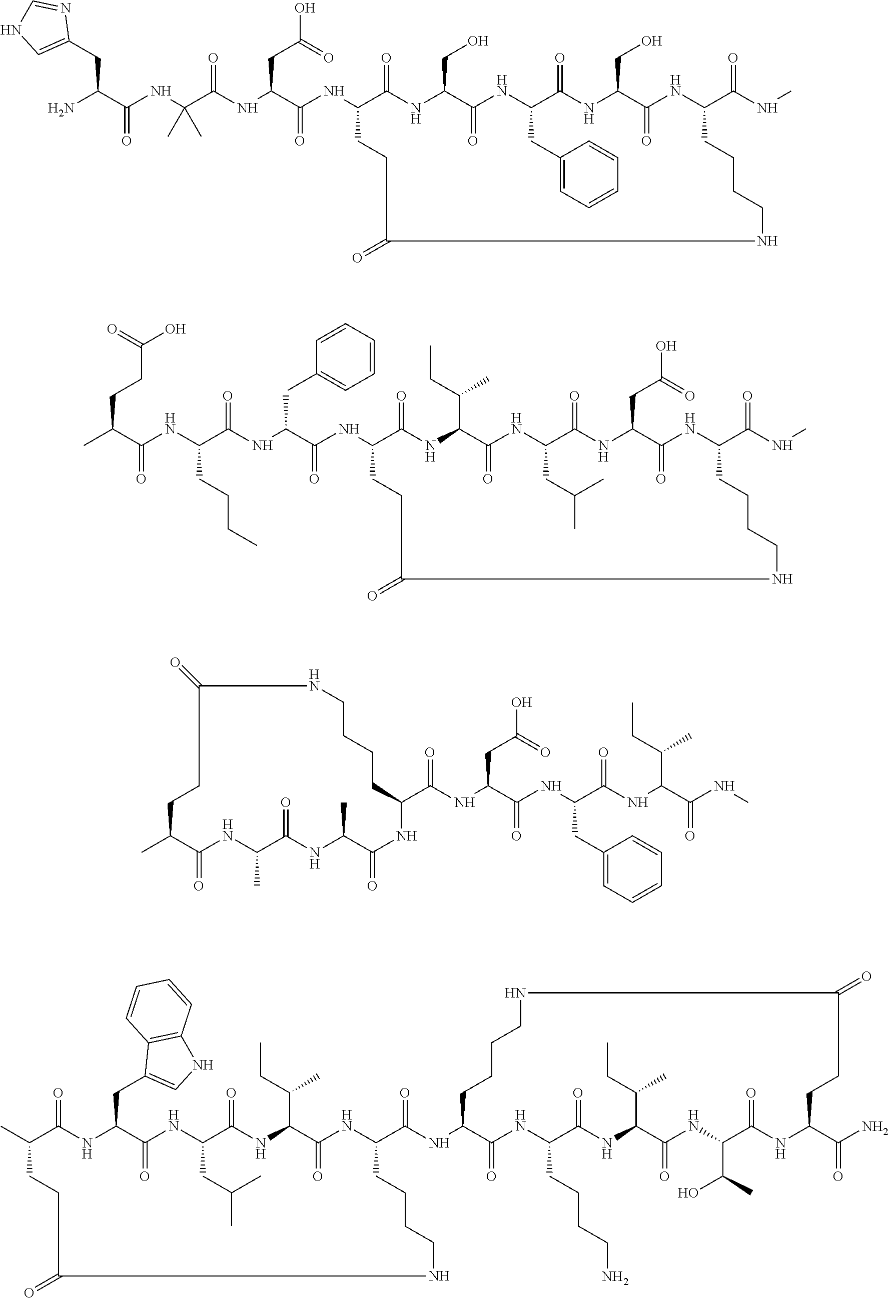

### Example 76

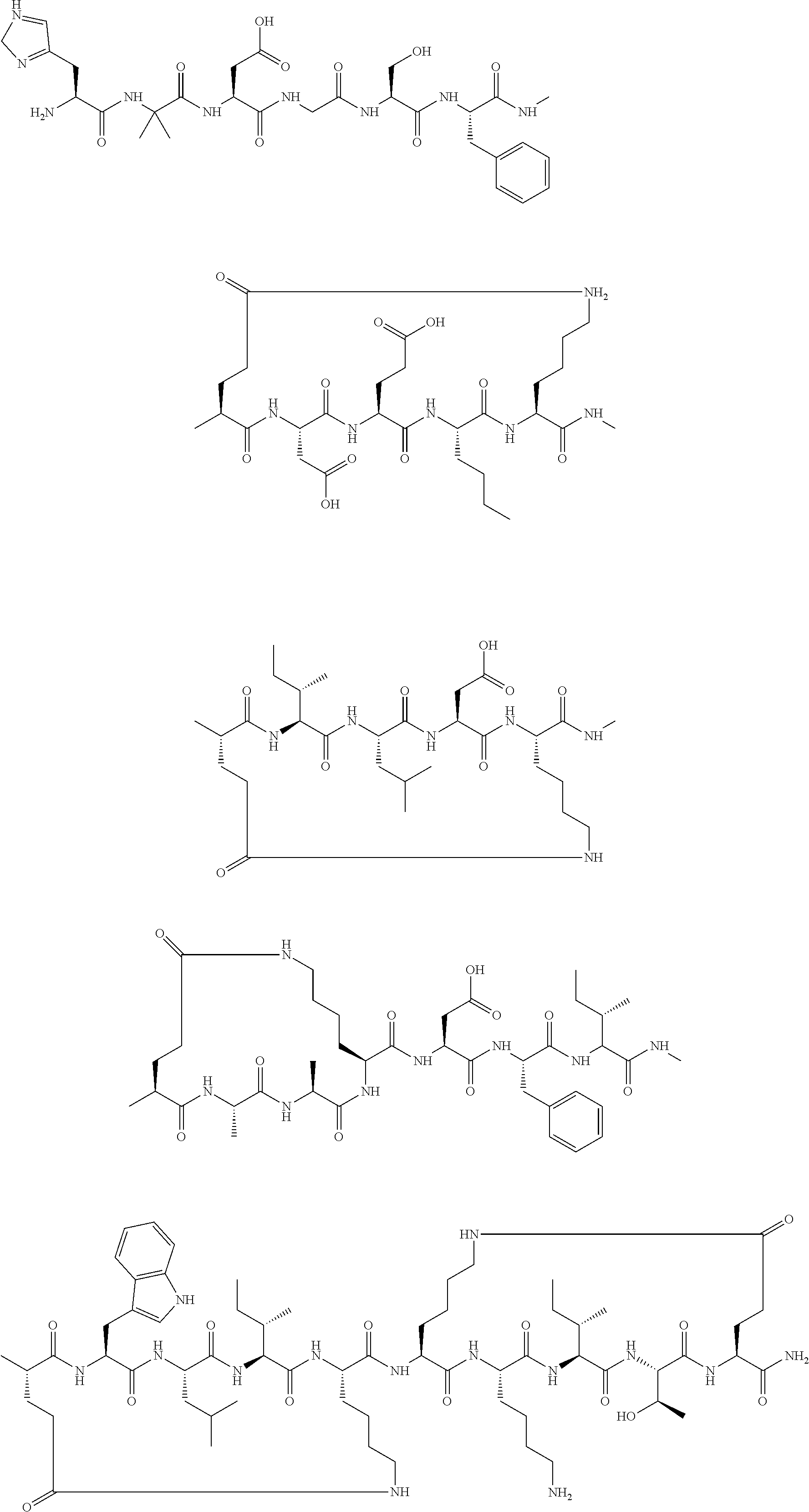

### Example 93

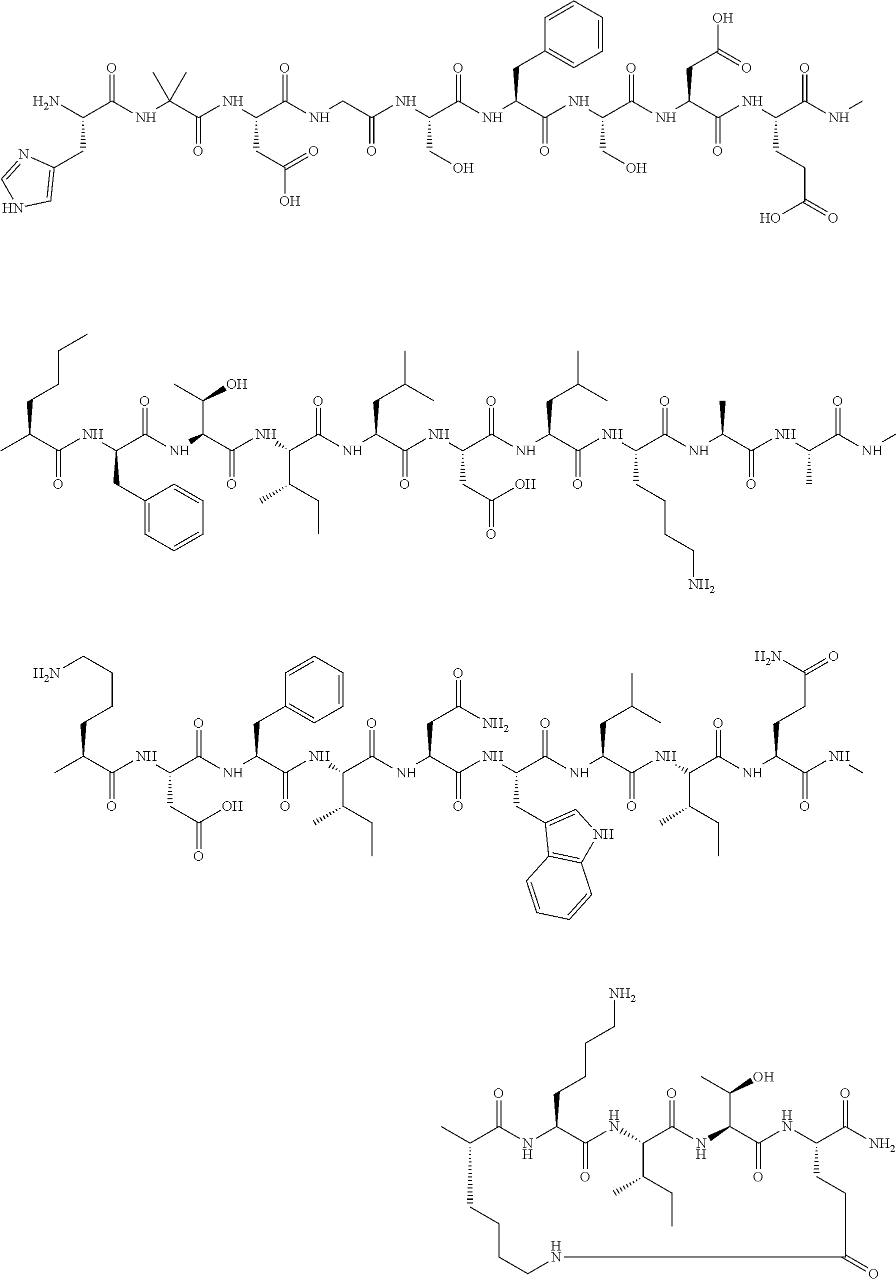

### Example 96

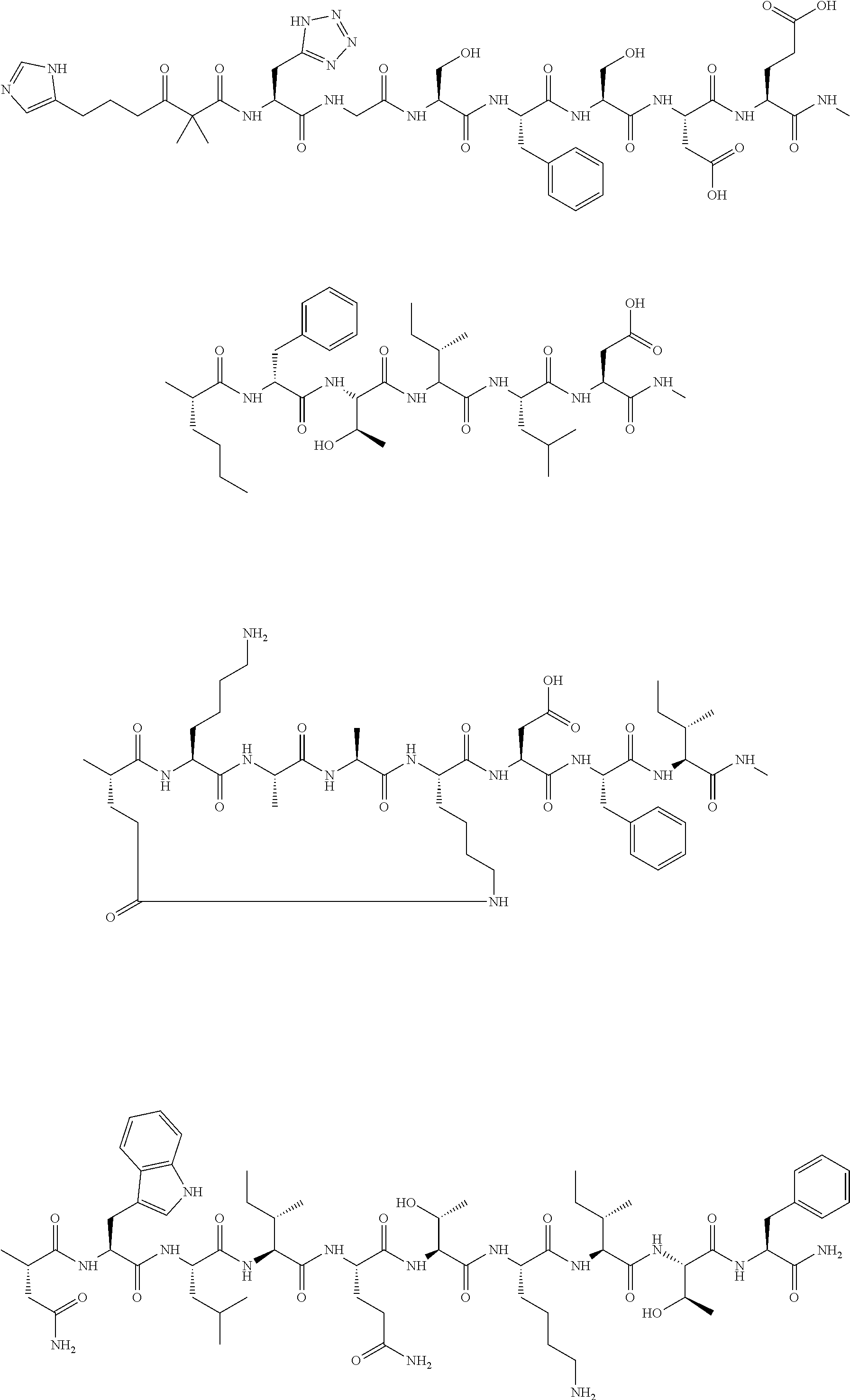

### Example 113

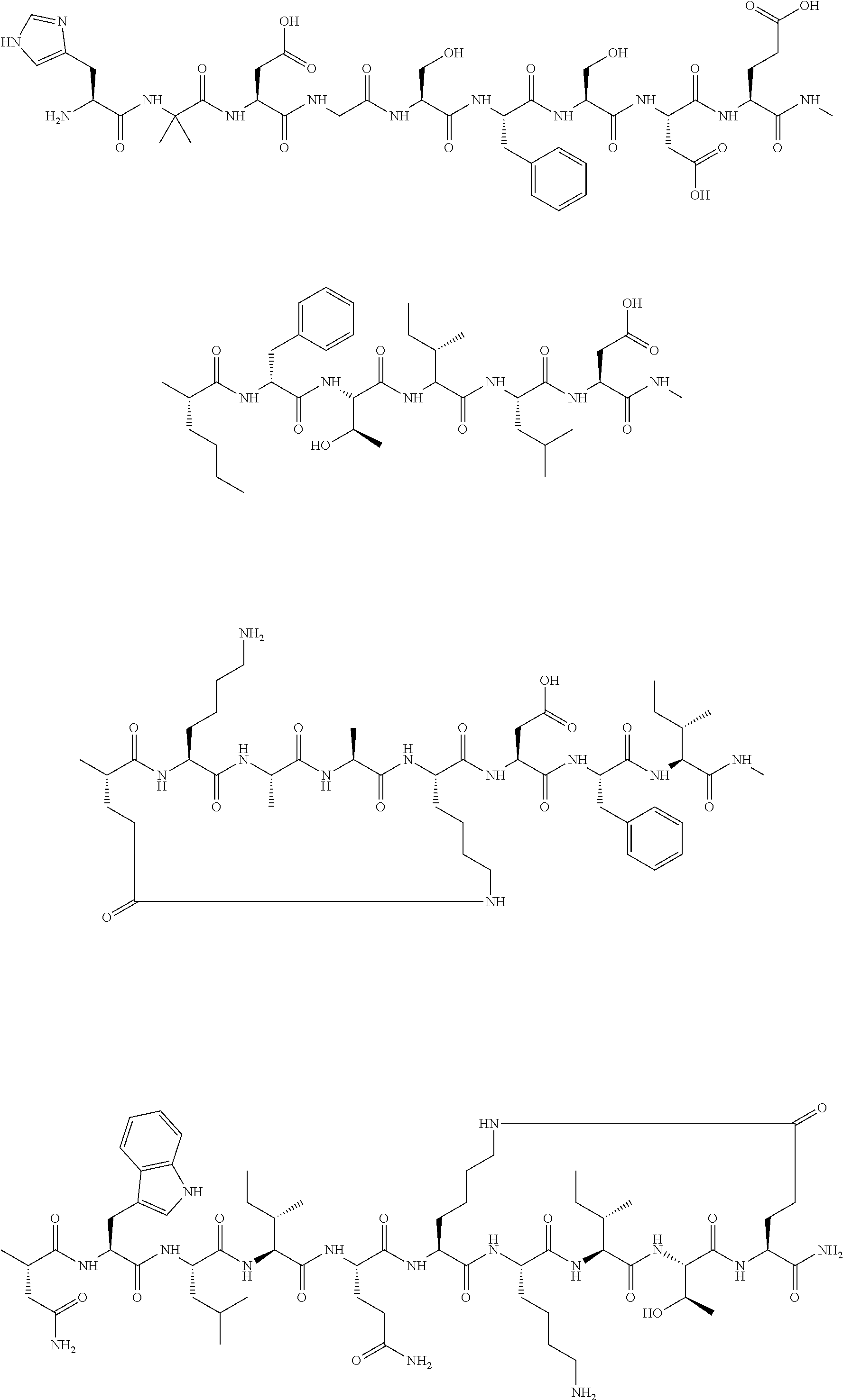

### Example 117

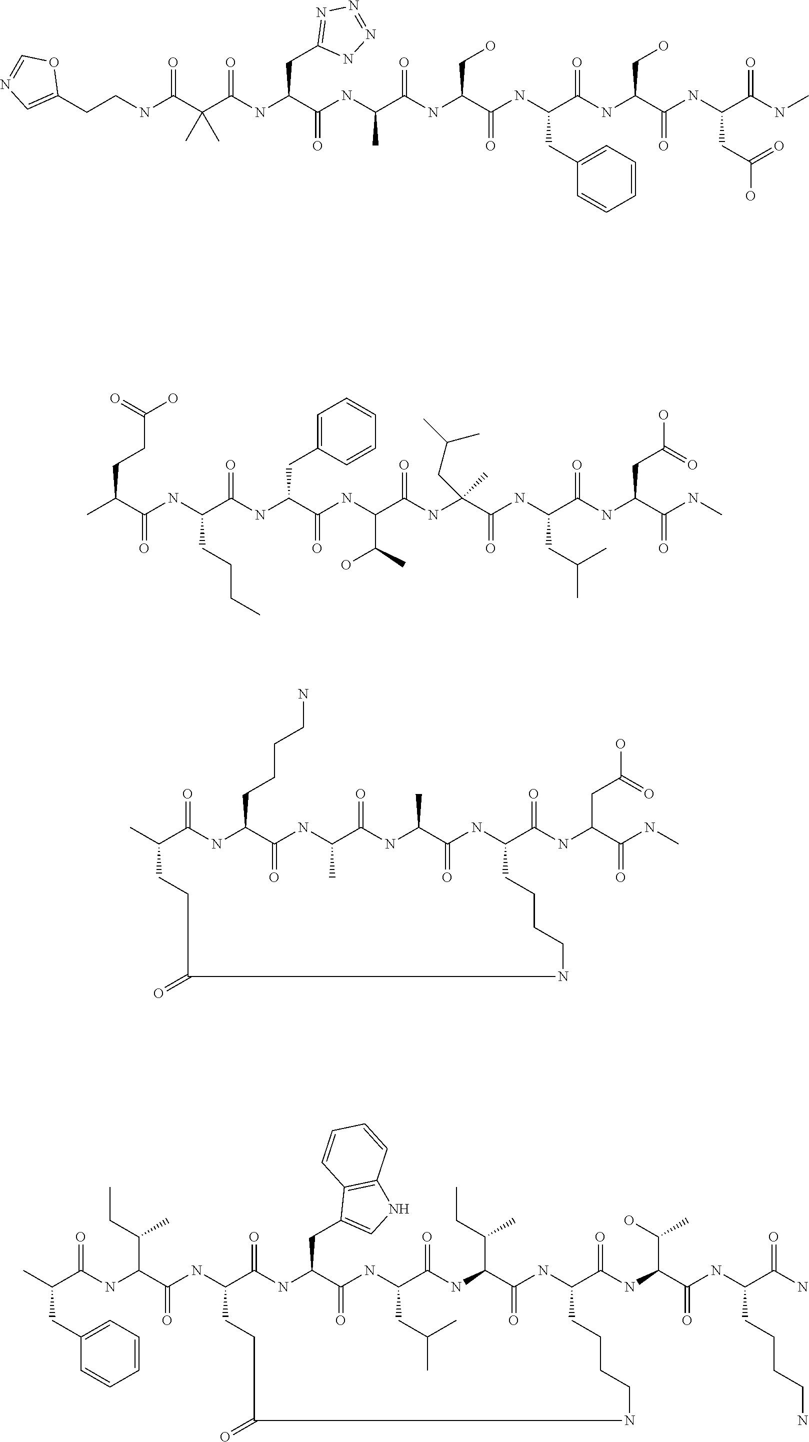

or a tautomer, salt or zwitterion thereof.

### General Procedures

Where no preparative routes are included, the relevant intermediate is commercially available. Commercial reagents were utilized without further purification. Room temperature (rt) refers to approximately 20-27° C. ¹H NMR spectra were recorded at 400 MHz on a Bruker instrument. Chemical shift values are expressed in parts per million (ppm), i.e. (8)-values. The following abbreviations are used for the multiplicity of the NMR signals: s=singlet, br=broad, d=doublet, t=triplet, q=quartet, quint=quintet, td=triplet of doublets, tt=triplet of triplets, qd=quartet of doublets, ddd=doublet of doublet of doublets, ddt=doublet of doublet of triplets, m=multiplet. Coupling constants are listed as J values, measured in Hz. NMR and mass spectroscopy results were corrected to account for background peaks. Chromatography refers to column chromatography performed using 60-120 mesh silica gel and executed under nitrogen pressure (flash chromatography) conditions.

### Analytical Methods

LCMS analysis of compounds was performed under electrospray conditions.

### LCMS Method A

Instruments: Waters Acquity UPLC, Waters 3100 PDA Detector, SQD; Column: Acquity HSS-T3, 1.8 micron, 2.1×100 mm; Gradient [time (min)/solvent B in A (%)]:0.00/10, 1.00/10, 2.00/15, 4.50/55, 6.00/90, 8.00/90, 9.00/10, 10.00/10; Solvents: solvent A=0.1% trifluoroacetic acid in water; solvent B=acetonitrile; Injection volume 1 μL; Detection wavelength 214 nm; Column temperature 30° C.; Flow rate 0.3 mL per min.

### LCMS Method B

LCMS: Agilent 1200 HPLC&6410B Triple Quad, Column: Xbridge C18 3.5 μm 2.1*30 mm. Gradient [time (min)/solvent B(%)]:0.0/10,0.9/80, 1.5/90,8.5/5, 1.51/10. (Solvent A=1 mL of TFA in 1000 ml Water; Solvent B=1 mL of TFA in 1000 mL of MeCN); Injection volume 5 L; UV detection 220 nm 254 nm 210 nm; Column temperature 25° C.; 1.0 mL/min.

### Analytical Method C

MS ion determined using LCMS method below under electrospray conditions, HPLC retention time (RT) determined using HPLC method below, purity >95% by HPLC unless indicated.

LCMS: Agilent 1200 HPLC&6410B Triple Quad, Column: Xbridge C18 3.5 μm 2.1*30 mm. Gradient [time (min)/solvent B(%)]:0.0/10,0.9/80, 1.5/90,8.5/5, 1.51/10. (Solvent A=1 mL of TFA in 1000 mL Water; Solvent B=1 mL of TFA in 1000 mL of MeCN); Injection volume 5 μL (may vary); UV detection 220 nm 254 nm 210 nm; Column temperature 25° C.; 1.0 mL/min. HPLC: Agilent Technologies 1200, Column: Gemini-NX C18 5 μm 110 A 150*4.6 mm. Gradient [time (min)/solvent B(%)]:0.0/30,20/60,20.1/90,23/90. (Solvent A=1 mL of TFA in 1000 ml Water; Solvent B=1 mL of TFA in 1000 mL of MeCN); Injection volume 5 μL (may vary); UV detection 220 nm 254 nm; Column temperature 25° C.; 1.0 mL/min.

### Analytical Method D

Instrument: Thermo Scientific Orbitrap Fusion; Column: Phenomenex Luna Omega C18 100 Å, 1.6 μm, 2.1×50 mm; Gradient [time (min)/solvent B in A (%)]:0.00/10, 0.30/10, 0.40/60, 1.10/90, 1.70/90, 1.75/10, 1.99/10, 2.00/10; Solvents: Solvent A=0.1% formic acid in water; Solvent B=0.1% formic acid in acetonitrile; Injection volume 5 μL; Column temperature 25° C.; Flow rate 0.8 mL/min.

### Synthesis of Intermediates and Compounds

The following examples are provided to illustrate preferred aspects of the invention and are not intended to limit the scope of the invention.

### Synthesis of Intermediates

All Fmoc-amino acids are commercially available except for intermediates 1, 2 and the Fmoc-cyclic peptide building blocks (intermediates 3 to 21)

### Synthesis of 2,2-dimethyl-3-oxo-3-((2-(1-trityl-1H-imidazol-4-yl)ethyl)amino)propanoic acid (Intermediate 1)

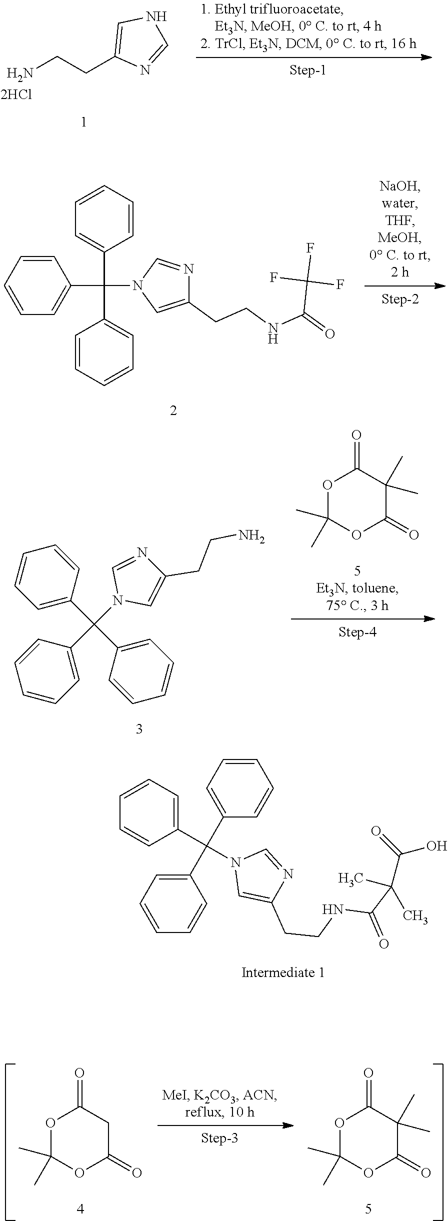

Step-1: Synthesis of 2,2,2-trifluoro-N-(2-(1-trityl-1H-imidazol-4-yl)ethyl)acetamide (2): To a solution of 2-(1H-imidazol-4-yl)ethan-1-amine dihydrochloride (1, 25.0 g, 136.6 mmol) in MeOH (100 mL), Et₃N (67 mL, 464.4 mmol) was added at rt and the reaction mixture was cooled to 0° C. A solution of ethyl trifluoroacetate (20 mL, 164.0 mmol) in MeOH (50 mL) was added to the reaction mixture over 30 min at 0° C. and the reaction mixture was stirred at rt for 4 h. This reaction mixture was diluted with dry DCM (200 mL) and Et₃N (60 mL, 409.8 mmol) and the reaction mixture was cooled to 0° C. Tr-CI (76 g, 273.2 mmol) was added portion wise and the resulting reaction mixture was stirred at rt for 16 h. After completion, the reaction mixture was quenched with water (300 mL) and the aq layer was extracted with chloroform (3×150 mL). The organic layers were combined, dried (Na₂SO₄) and concentrated in vacuo. The crude residue was triturated with n-hexane to give 2,2,2-trifluoro-N-(2-(1-trityl-1H-imidazol-4-yl)ethyl)acetamide (2, 50.10 g, 81%) as a white solid.

MS (ESI+ve): 450

¹H-NMR (400 MHZ; CDCl₃): 82.75 (t, J=5.9 Hz, 2H), 3.60-3.65 (m, 2H), 6.61 (s, 1H), 7.08-7.15 (m, 6H), 7.31-7.38 (m, 9H), 7.40 (s, 1H), 8.41 (bs, 1H).

Step-2: Synthesis of 2-(1-trityl-1H-imidazol-4-yl)ethan-1-amine (3): To a solution of 2,2,2-trifluoro-N-(2-(1-trityl-1H-imidazol-4-yl)ethyl)acetamide (2, 50.0 g, 111.3 mmol) in THF (150 mL) and MeOH (180 mL), NaOH (22.0 g, 556.7 mmol) in water (100 mL) was slowly added at 0° C. and the reaction mixture was stirred at room temperature for 2 h. After completion, the reaction mixture was quenched with water (300 mL) and the aq layer was extracted with chloroform (3×150 mL). The organic layers were combined, dried (Na₂SO₄) and concentrated in vacuo to give 2-(1-trityl-1H-imidazol-4-yl)ethan-1-amine (3, 34.0 g, 86%) as a yellowish sticky solid. The crude residue was used for the next step without further purification.

MS (ESI+ve): 354

¹H-NMR (400 MHZ; CDCl₃): δ 1.53 (bs, 2H), 2.65 (t, J=6.5 Hz, 2H), 2.95 (t, J=6.5 Hz, 2H), 6.58 (s, 1H), 7.11-7.16 (m, 6H), 7.28-7.38 (m, 10H).

Step-3: Synthesis of 2,2,5,5-tetramethyl-1,3-dioxane-4,6-dione (5): To a solution of 2,2-dimethyl-1,3-dioxane-4,6-dione (4, 20.0 g, 138.8 mmol) in ACN (200 mL), K₂CO₃ (96 g, 694.0 mmol) and Mel (26 mL, 416.6 mmol) were added at rt and reaction mixture was refluxed for 10 h. After completion, the reaction mixture was cooled to room temperature, filterd through a pad of celite, washed with EtOAc (3×50 mL). The organic layer was washed with 10% aq Na₂S₂O₃ (100 mL), dried, (Na₂SO₄) and concentrated in vacuo to give 2,2,5,5-tetramethyl-1,3-dioxane-4,6-dione (5, 21 g, 88%) as a yellow solid. The crude residue was used for the next step without further purification.

1H-NMR (400 MHZ; CDCl₃): δ 1.63 (s, 6H), 1.73 (s, 6H).

Step-4: Synthesis of 2,2-dimethyl-3-oxo-3-((2-(1-trityl-1H-imidazol-4-yl)ethyl)amino) propanoic acid (Intermediate 1): A solution of 2-(1-trityl-1H-imidazol-4-yl)ethan-1-amineto (3, 8.0 g, 22.6 mmol) and Et₃N (16.0 mL, 113.0 mmol) in toluene (100 mL) was added drop wise over 60 min to a solution of 2,2,5,5-tetramethyl-1,3-dioxane-4,6-dione (5, 5.8 g, 29.76 mmol) in toluene (50 mL) at 75° C. The reaction mixture was further stirred at same temperature was 3 h. After completion, the reaction mixture was concentrated in vacuo. The residue was dissolved in chloroform (100 mL) and washed with 10% aq citric acid (pH˜6-6.5). The organic layer was dried (Na₂SO₄) and concentrated in vacuo. The crude residue obtained was triturated with hot chloroform (150 mL) and n-hexane (75 mL) and the suspension was stirred at rt for 16 h. The solid was filtered, washed with chloroform:n-hexane (1:1, 2×50 mL) and dried in vacuo to give 2.2-dimethyl-3-oxo-3-((2-(1-trityl-1H-imidazol-4-yl)ethyl)amino)propanoic acid (Intermediate 1, 6.8 g, 64%) as a white solid.

LCMS (Method A): m/z 468 [M+H]⁺ (ES⁺), at 5.38 min, 99.31%

¹H-NMR (400 MHZ; DMSO-d₆): δ 1.21 (s, 6H), 2.57 (t, J=6.8 Hz, 2H), 3.22-3.27 (m, 2H), 6.66 (s, 1H), 7.06-7.11 (m, 6H), 7.28 (s, 1H), 7.35-7.42 (m, 8H), 7.64 (t, J=5.4 Hz, 1H), 8.31 (s, 1H), 12.44 (bs, 1H).

### Synthesis of (S)-2-((((9H-fluoren-9-yl)methoxy)carbonyl)amino)-3-(2-trityl-2H-tetrazol-5-yl)propanoic acid (Intermediate 2)

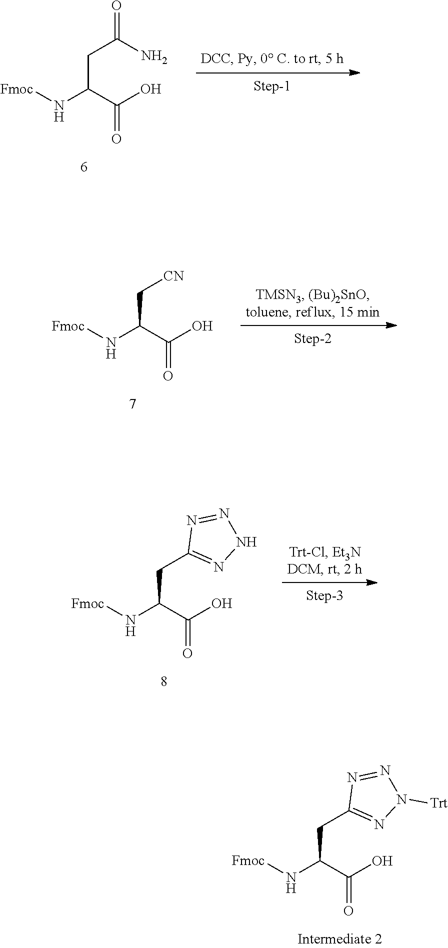

Step-1: Synthesis of (S)-2-((((9H-fluoren-9-yl)methoxy)carbonyl)amino)-3-cyanopropanoic acid (7): To a suspension of (((9H-fluoren-9-yl)methoxy)carbonyl)-L-asparagine (7, 50.0 g, 423.7 mmol) in pyridine (200 mL) was added DCC (34.0 g, 466.1 mmol) at 0° C. and the reaction mixture was stirred at room temperature for 5 h. The reaction mixture was carefully quenched with aq. 2N HCl till pH became acidic and extracted with diethyl ether (3×500 mL). The organic layers were combined and washed with brine, dried (Na₂SO₄) and concentrated in vacuo. The residue was triturated with pentane to give (S)-2-((((9H-fluoren-9-yl)methoxy)carbonyl)amino)-3-cyanopropanoic acid (7, 96 g, 68%) as a white solid.

MS (ESI−ve): 335.

¹H-NMR (400 MHZ; DMSO-d₆): δ 2.85-3.05 (m, 2H), 4.22-4.39 (m, 4H), 7.33 (t, J=7.6 Hz, 2H), 7.42 (t, J=7.6 Hz, 2H), 7.72 (d, J=7.2 Hz, 2H), 7.90 (d, J=7.6 Hz, 2H), 8.09 (d, J=8.4 Hz, 1H).

Step-2: Synthesis of (S)-2-((((9H-fluoren-9-yl)methoxy)carbonyl)amino)-3-(2H-tetrazol-5-yl)propanoic acid (8): To a suspension of (S)-2-((((9H-fluoren-9-yl)methoxy)carbonyl)amino)-3-cyanopropanoic acid (7, 48.0 g, 142.8 mmol) in toluene (50 mL), dibutyltin oxide (21.0 g, 85.6 mmol) was added and the reaction mixture was stirred for 15 min. To this reaction mixture trimethylsilyl azide (61 mL, 422.8 mmol) was added and reaction mixture was refluxed at 120° C. for 15 min. After cooling the reaction mixture to room temperature, the resultant solid formed was filtered and washed with diethyl ether. The solid residue was triturated with 5% MeOH/DCM (500 mL) to give (S)-2-((((9H-fluoren-9-yl)methoxy)carbonyl)amino)-3-(2H-tetrazol-5-yl)propanoic acid (8, 32.5 g, 60%) as an off white solid.

MS (ESI+ve): 380

¹H-NMR (400 MHZ; DMSO-d₆): 83.22-3.41 (m, 2H), 4.18-4.28 (m, 3H), 4.41-4.48 (m, 1H), 7.31 (t, J=7.2 Hz, 2H), 7.41 (t, J=7.2 Hz, 2H), 7.65 (t, J=7.6 Hz, 2H), 7.77 (d, J=7.6 Hz, 1H), 7.88 (d, J=7.6 Hz, 2H).

Step-3: Synthesis of (S)-2-((((9H-fluoren-9-yl)methoxy)carbonyl)amino)-3-(2-trityl-2H-tetrazol-5-yl)propanoic acid (Intermediate 2): To a solution of (S)-2-((((9H-fluoren-9-yl)methoxy)carbonyl)amino)-3-(2H-tetrazol-5-yl)propanoic acid (8, 12×5 g, 12×13.0 mmol) in DCM (12×45 mL), Et₃N (12×5.6 mL, 12×39.0 mmol) was added at 0° C. After stirring for 5 min, trityl chloride (12×4.0 g, 12×14.0 mmol) was added and the reaction mixture was stirred at the same temperature for 2 h. Reaction mixture was quenched with water (50 mL) and extracted with DCM (2×100 mL) (12 times). The organic layers were combined and washed with brine, dried (Na₂SO₄) and concentrated in vacuo. The residue was purified by flash column chromatography [normal phase, silica gel (100-200 mesh), gradient 1% to 5% methanol in DCM] to give (S)-2-((((9H-fluoren-9-yl)methoxy)carbonyl)amino)-3-(2-trityl-2H-tetrazol-5-yl)propanoic acid (Intermediate 2, 41 g, 41%) as a white solid.

LCMS (Method A): m/z 620 [M−H]⁺ (ES⁻), at 5.99 min, 86.85%

¹H-NMR (400 MHZ; CDCl₃): δ 3.44-3.62 (m, 2H), 4.12-4.20 (m, 1H), 4.25-4.32 (m, 1H), 4.36-4.44 (m, 1H), 4.82-4.88 (m, 1H), 7.02-7.12 (m, 6H), 7.24-7.32 (m, 11H), 7.34-7.42 (m, 2H), 7.44-7.48 (m, 1H), 7.49-7.58 (m, 2H), 7.74 (d, J=6.6 Hz, 2H).

Used in solid phase peptide synthesis without further purification

### Method for the Synthesis of Fmoc-Cyclic Peptide Building Blocks, Exemplified by the Synthesis of Intermediate 8, Fmoc-[Asp-Ile-Leu-Lys]

1) Add DCM to the vessel containing CTC Resin (3 mmol, 3 g, 1.0 mmol/g) and Fmoc-Lys(Alloc)-OH (1.35 g, 3 mmol, 1 eq) agitate with N₂ bubbling.
2) Add DIEA (4.0 eq) dropwise and agitate with N₂ bubbling for 2 hours
3) Add MeOH (3 mL) and agitate with N₂ bubbling for 30 min.
4) Drain and wash resin with DMF (5 times, drain between each wash).
5) A solution of 20% piperidine in DMF was added and resin agitated with N₂ bubbling for 30 min.
6) Drain and wash with DMF (5 times, drain between each wash).
7) Add Fmoc-amino acid solution (3.0 equivalents in DMF) and agitate with N₂ bubbling for 30 seconds, then add activation buffer (HBTU (2.85 equivalents) and DIEA (6 equivalents) in DMF), agitate with N₂ bubbling for 1 hour.
8) The coupling reaction was monitored by ninhydrin test
9) If required repeat steps 6 to 8 for same amino acid coupling if inefficient coupling occurs
10) Repeat steps 3 to 8 for next amino acid coupling.

Note: for the acids in the table below different protecting groups and / or coupling agents were used

| Amino acid site | | |
|---|---|---|
| from resin | Materials | Coupling reagents |
| 1 | Fmoc-Lys(Alloc)-OH (1.0 eq) | DIEA (4.0 eq) |
| 4 | Fmoc-Glu(OAll)-OH (3.0 eq) | HBTU (2.85 eq) and |
| | | DIEA (6.0 eq) |

### Peptide Sidechain Deprotection Cyclisation:

1) Add DCM to the resin and agitate with N₂ bubbling, then add PhSiH₃ (10 eq), Pd(PPh₃)₄ (0.2 eq) agitate with N₂ for 15 mins for 3 times.
2) The resin was washed with DCM three times and then DMF three times.
3) The resin was washed with 0.5% Sodium diethyldithiocarbamate trihydrate DMF and 0.5% DIEA in DMF for ten times.
4) HATU (2 eq) and DIEA (4 eq) was added to the resin in DMF and agitate with N₂ bubbling for 1 hour.
3) The resin was washed with MeOH three times and dried in vacuo.
4) The resin was added to a solution of 20% HFIP/80% DCM and stir for 30 mins, filtered and repeated.
5) Organic layers were combined and the solvent was removed in vacuo.
6) The peptide was washed with H₂O twice.
7) Peptide re-dissolved and lyophilize to give Intermediate 8 (1.5 g, 55.6% yield) as a solid.

Intermediates 3 to 21 were synthesized using the above procedure, analytical data is given below:

| Intermediate | Structure | ESI (LCMS Method B) |
|---|---|---|
| 3 | 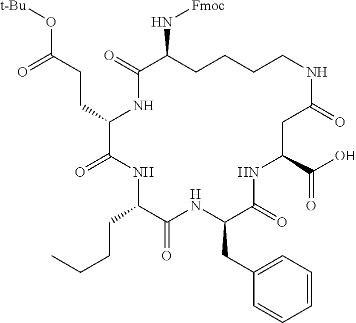 | Mass not observed |
| 4 | 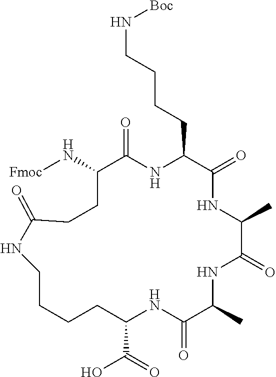 | 833.3 [M-tBu + MeCN]⁺ |
| 5 | 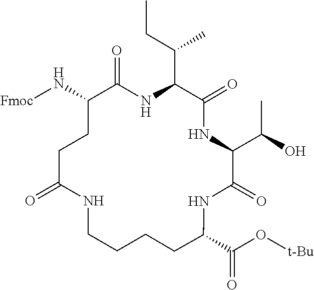 | Mass not observed |
| 6 | 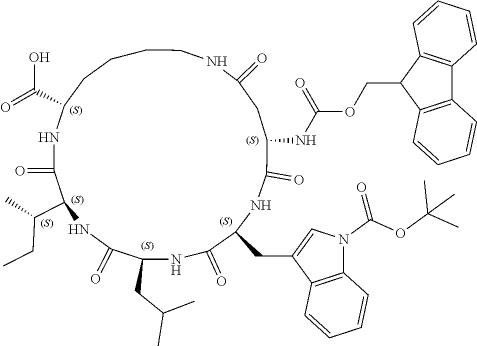 | 991.7 [M − H]⁻ |
| 7 | 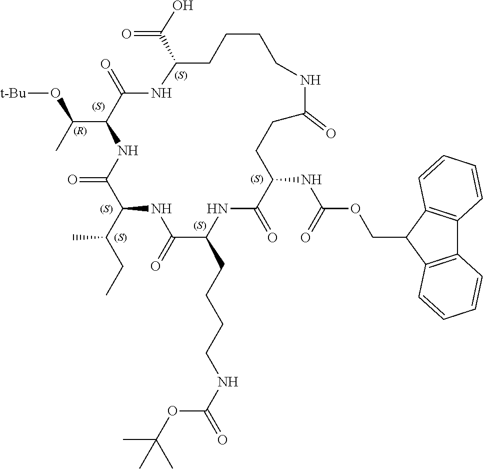 | 977.8 [M − H]⁻ |
| 8 | 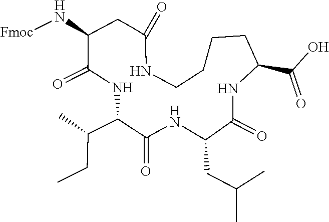 | 692.1 [M + H]⁺ |
| 9 | 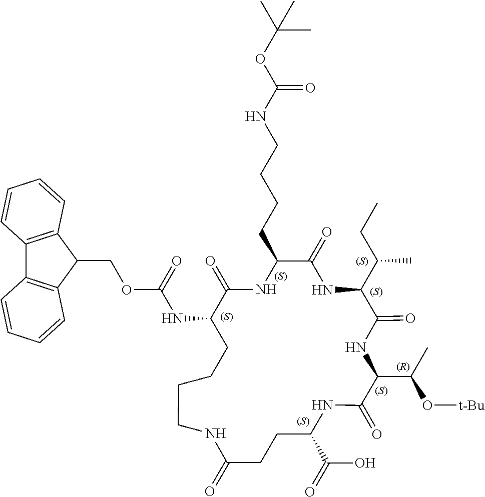 | 977.8 [M − H]⁻ |
| 10 | 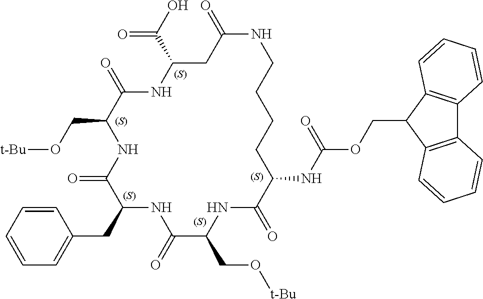 | 898.7 [M − H]⁻ |
| 11 | 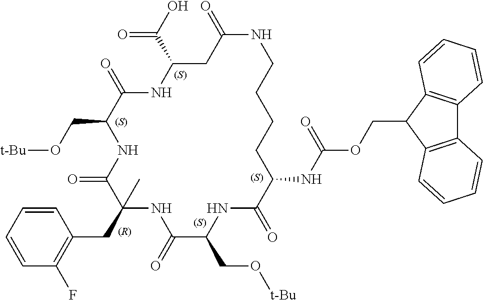 | 930.7 [M − H]⁻ |
| 12 | 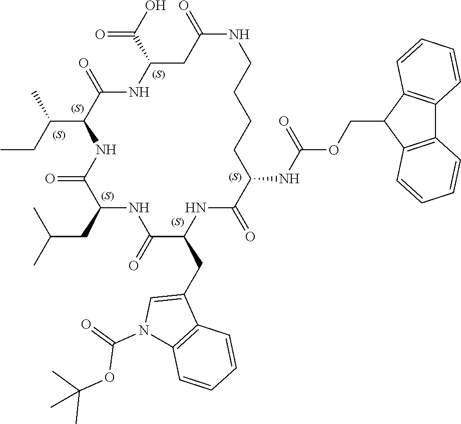 | 977.7 [M − H]⁻ |
| 13 | 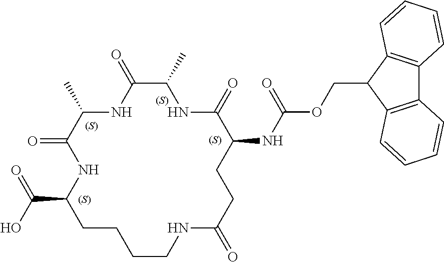 | 621.7 [M − H]⁻ |
| 14 | 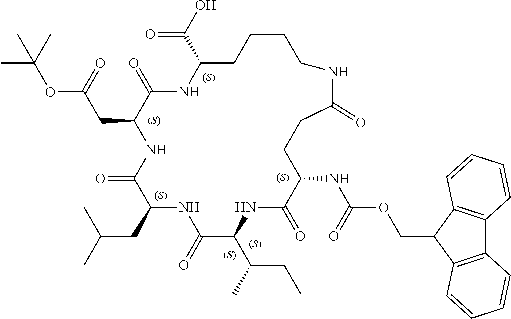 | 876.8 [M − H]⁻ |
| 15 | 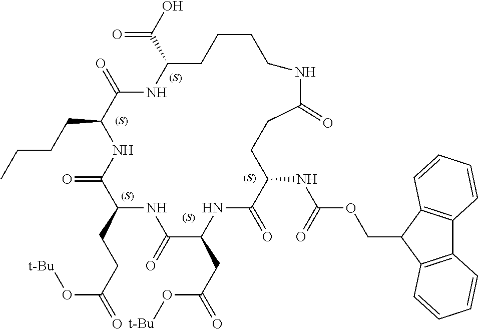 | 948.7 [M − H]⁻ |
| 16 | 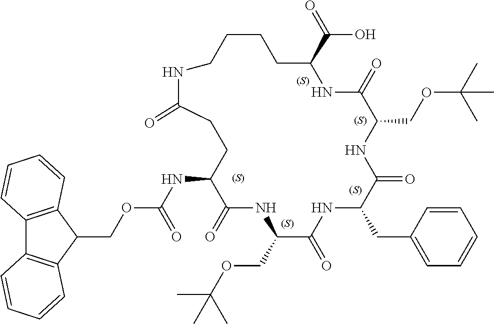 | 912.7 [M − H]⁻ |
| 17 | 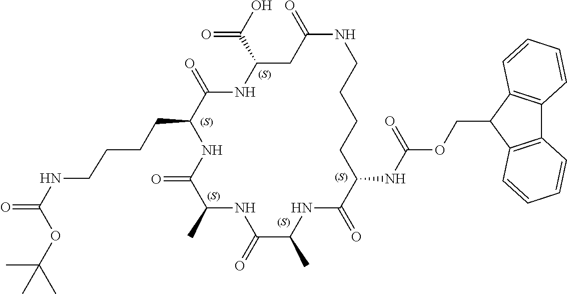 | 835.6 [M − H]⁻ |
| 18 | 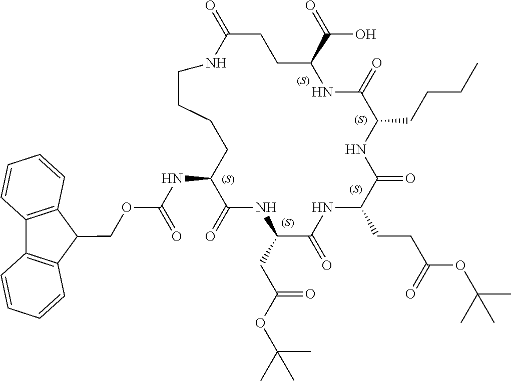 | 948.7 [M − H]⁻ |
| 19 | 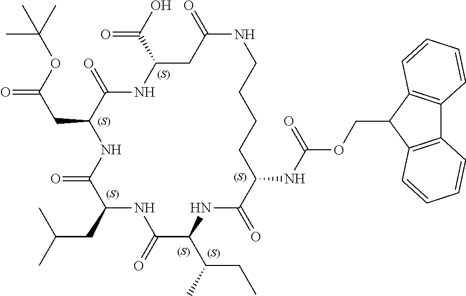 | 862.7 [M − H]⁻ |
| 20 | 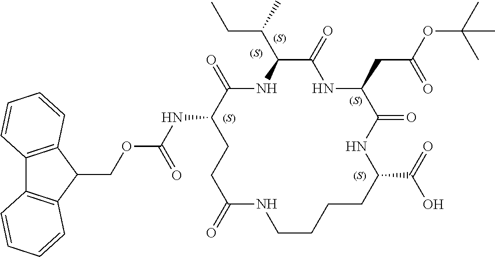 | 749.6 [M-tBu + MeCN]⁺ |
| 21 | 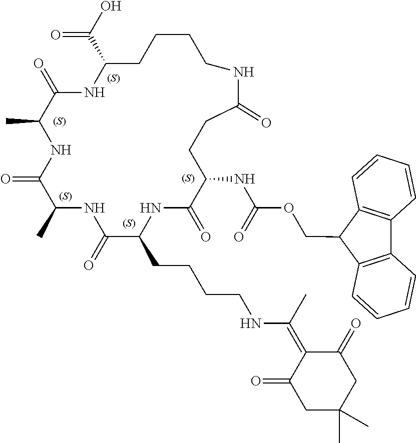 | 913.7 [M − H]⁻ |

### Synthesis of Examples 1-81

Standard Fmoc solid phase peptide synthesis (SPPS) was used to synthesize the peptides which were then cleaved from the resin and purified.

### General Method for Peptide Synthesis:

The peptide was synthesized using standard Fmoc chemistry.

### Method a—Exemplified by the Synthesis of Example 1

### Peptide Synthesis

1) Add DCM to the vessel containing Rink Amide MBHA Resin (sub: 0.35 mmol/g, 0.15 mmol, 0.42 g) and swell for 2 hours.
2) Drain and then wash with DMF (5 times, drain between each wash).
3) A solution of 20% piperidine in DMF was added agitate with N₂ bubbling for 30 min.
4) Drain and wash with DMF (5 times, drain between each wash).
5) Add Fmoc-amino acid solution (3.0 equivalents in DMF) and mix for 30 seconds, then add activation buffer (HBTU (2.85 equivalents) and DIEA (6 equivalents) in DMF), agitate with N₂ bubbling for 1 hour.
6) The coupling reaction was monitored by ninhydrin test
7) If required repeat steps 4 to 6 for same amino acid coupling if inefficient coupling occurs
8) Repeat steps 2 to 6 for next amino acid coupling.
   Note: for the acids in the table below different protecting groups and / or coupling agents were used

| Step | Materials | Coupling reagents |
|---|---|---|
| 1 | Intermediate 5 (2.0 eq) | DIC (2.0 eq) and HOBT (2.0 eq) |
| 11 | Intermediate 4 (2.0 eq) | DIC (2.0 eq) and HOBT (2.0 eq) |
| 15 | Intermediate 3 (2.0 eq) | DIC (2.0 eq) and HOBT (2.0 eq) |
| 20 | Intermediate 2 (2.0 eq) | DIC (2.0 eq) HOBT (2.0 eq) |
| 21 | Intermediate 1(1.5 eq) | DIC (1.5 eq) HOBT (1.5 eq) |

9) The resin was washed with DMF five times and MeOH three times and dried in vacuo.

### Peptide Cleavage and Purification:

1) Add cleavage buffer (92.5% TFA/2.5% EDT/2.5% TIS/2.5% H₂O) to the flask containing the side chain protected peptide on resin at room temperature and stir for 3 hours.
2) Filter and collect the peptide solution.
3) The peptide is precipitated with cold tert-butyl methyl ether and centrifuged (3 min at 3000 rpm).
4) Residue washed with tert-butyl methyl ether (2 times).
5) Crude peptide dried under vacuum for 2 hours.)
6) The crude peptide was purified by prep-HPLC. Prep-HPLC Conditions: Instrument: Gilson 281. Solvent: A—0.1% TFA in H₂O, B—acetonitrile, Column: Luna C18 (200×25 mm; 10 μm) and Gemini C18 (150*30 mm; 5 μm) in series. Gradient [time (min)/solvent B (%)]:0.0/25, 60.0/55, 60.1/90, 70/90, 70.1/10, at 20 mL/min with UV detection (wave length=215/254 nm). Residue was re-purified by prep-HPLC. Prep-HPLC Conditions: Instrument: Gilson 281. Solvent: A—0.08% NH₄HCO₃ in H₂O, B—acetonitrile, Column: Luna C18 (200×25 mm; 10 μm) and Gemini C18 (150*30 mm; 5 μm) in series. Gradient [time (min)/solvent B (%)]:0.0/20, 60.0/55, 60.1/90, 70/90, 70.1/10, at 20 mL/min with UV detection (wave length=215/254 nm) and then lyophilized to give Example 1 ((65.7 mg, 11.6% yield).

TABLE 2
HRMS and LCMS properties of purified peptides represented by Examples 1-81

| | HRMS | LCMS/HPLC |
|---|---|---|
| Example | (Analytical Method D) | (Analytical Method B) |
| 1 | HRMS (HESI/FT) m/z: [M + 3H]³⁺ Calcd for | m/z 1259.9 [M + 3H]³⁺, R_{T} = 12.66 min |
| | C175H267N46O48 3777.9631; Found 1259.6610 | |
| 2 | ND | m/z 1264.7 [M + 3H]³⁺, R_{T} = 12.64 min |
| 3 | HRMS (HESI/FT) m/z: [M + 3H]³⁺ Calcd for | m/z 873.4 [M + 4H]⁴⁺, R_{T} = 12.54 min |
| | C165H251N42O42 3489.8562; Found 1164.2959 | |
| 4 | HRMS (HESI/FT) m/z: [M + 3H]³⁺ Calcd for | m/z 953.1 [M + 4H]⁴⁺, R_{T} = 10.95 min |
| | C178H273N44O49 3807.9988; Found 1270.3432 | |
| 5 | HRMS (HESI/FT) m/z: [M + 3H]³⁺ Calcd for | m/z 871.4 [M + 4H]⁴⁺, R_{T} = 11.41 min |
| | C165H252N39O44 3480.8445; Found 1161.2913 | |
| 6 | HRMS (HESI/FT) m/z: [M + 3H]³⁺ Calcd for | m/z 1271.2 [M + 3H]³⁺, R_{T} = 12.61 min |
| | C182H279N46O44 3810.0774; Found 1271.0360 | |
| 7 | HRMS (HESI/FT) m/z: [M + 4H]⁴⁺ Calcd for | m/z 949.7 [M + 4H]⁴⁺, R_{T} = 11.55 min |
| | C183H281N45O43 3793.0872; Found 949.5311 | |
| 8 | HRMS (HESI/FT) m/z: [M + 3H]³⁺ Calcd for | m/z 955.6 [M + 4H]⁴⁺, R_{T} = 12.99 min |
| | C178H271N46O48 3817.9944; Found 1273.6759 | |
| 9 | HRMS (HESI/FT) m/z: [M + 4H]⁴⁺ Calcd for | m/z 1127.6 [M + 4H]⁴⁺, R_{T} = 9.04 min |
| | C211H329N49O60 4505.3887; Found 1127.3612 | |
| 10 | HRMS (HESI/FT) m/z: [M + 4H]⁴⁺ Calcd for | m/z 1134.4 [M + 4H]⁴⁺, R_{T} = 14.68 min |
| | C213H334N50O59 4532.436; Found 1134.1217 | |
| 11 | HRMS (HESI/FT) m/z: [M + 3H]³⁺ Calcd for | m/z 952.0 [M + 4H]⁴⁺, R_{T} = 12.88 min |
| | C177H269N46O48 3803.9788; Found 1269.0047 | |
| 12 | HRMS (HESI/FT) m/z: [M + 3H]³⁺ Calcd for | m/z 952.2 [M + 4H]⁴⁺, R_{T} = 13.30 min |
| | C177H269N46O48 3803.9788; Found 1269.0046 | |
| 13 | HRMS (HESI/FT) m/z: [M + 3H]³⁺ Calcd for | m/z 972.9 [M + 4H]⁴⁺, R_{T} = 15.46 min |
| | C181H274N47O49 3887.0159; Found 1296.6830 | |
| 14 | HRMS (HESI/FT) m/z: [M + 3H]³⁺ Calcd for | m/z 952.1 [M + 4H]⁴⁺, R_{T} = 12.94 min |
| | C177H269N46O48 3803.9788; Found 1269.0040 | |
| 15 | HRMS (HESI/FT) m/z: [M + 3H]³⁺ Calcd for | m/z 960.0 [M + 4H]⁴⁺, R_{T} = 13.02 min |
| | C178H270FN46O48 3835.9851; Found 1279.6729 | |
| 16 | HRMS (HESI/FT) m/z: [M + 3H]³⁺ Calcd for | m/z 955.4 [M + 4H]⁴⁺, R_{T} = 14.16 min |
| | C178H271N46O48 3817.9944; Found 1273.6759 | |
| 17 | HRMS (HESI/FT) m/z: [M + 3H]³⁺ Calcd for | m/z 881.2 [M + 4H]⁴⁺, R_{T} = 12.15 min |
| | C166H253N42O43 3519.8667; Found 1174.2989 | |
| 18 | HRMS (HESI/FT) m/z: [M + 3H]³⁺ Calcd for | m/z 1281.2 [M + 3H]³⁺, R_{T} = 11.48 min |
| | C179H274FN44O49 3840.0051; Found 1281.0113 | |
| 19 | HRMS (HESI/FT) m/z: [M + 3H]³⁺ Calcd for | m/z 1285.8 [M + 4H]⁴⁺, R_{T} = 11.48 min |
| | C180H276FN44O49 3854.0208; Found 1285.6836 | |
| 20 | HRMS (HESI/FT) m/z: [M + 4H]⁴⁺ Calcd for | m/z 1140.2 [M + 4H]⁴⁺, R_{T} = 13.89 min |
| | C214H336FN47O61 4555.4307; Found 1139.8694 | |
| 21 | HRMS (HESI/FT) m/z: [M + 3H]³⁺ Calcd for | m/z 1171.6 [M + 4H]⁴⁺, R_{T} = 7.67 min |
| | C166H254N39O45 3510.8552; Found 1171.2946 | |
| 22 | HRMS (HESI/FT) m/z: [M + 3H]³⁺ Calcd for | m/z 955.3 [M + 4H]⁴⁺, R_{T} = 11.99 min |
| | C178H272N47O47 3817.0105; Found 1273.3478 | |
| 23 | HRMS (HESI/FT) m/z: [M + 3H]³⁺ Calcd for | m/z 969.7 [M + 4H]⁴⁺, R_{T} = 10.03 min |
| | C180H271N46O50 3873.9841; Found 1292.3397 | |
| 24 | HRMS (HESI/FT) m/z: [M + 3H]³⁺ Calcd for | m/z 962.3 [M + 4H]⁴⁺, R_{T} = 12.34 min |
| | C180H276N47O47 3845.0417; Found 1282.6919 | |
| 25 | HRMS (HESI/FT) m/z: [M + 3H]³⁺ Calcd for | m/z 964.3 [M + 4H]⁴⁺, R_{T} = 12.66 min |
| | C181H277FN43O49 3853.0254; Found 1285.3529 | |
| 26 | HRMS (HESI/FT) m/z: [M + 3H]³⁺ Calcd for | m/z 960.6 [M + 4H]⁴⁺, R_{T} = 12.46 min |
| | C180H275FN43O49 3839.0098; Found 1280.6810 | |
| 27 | HRMS (HESI/FT) m/z: [M + 3H]³⁺ Calcd for | m/z 1176.0 [M + 3H]³⁺, R_{T} = 11.86 min |
| | C167H257N40O44 3523.8867; Found 1175.6413 | |
| 28 | HRMS (HESI/FT) m/z: [M + 3H]³⁺ Calcd for | m/z 1176.3 [M + 3H]³⁺, R_{T} = 12.22 min |
| | C167H256N39O45 3524.8708; Found 1175.9685 | |
| 29 | HRMS (HESI/FT) m/z: [M + 3H]³⁺ Calcd for | m/z 1161.3 [M + 3H]³⁺, R_{T} = 14.38 min |
| | C165H249N38O45 3479.813; Found 1160.9532 | |
| 30 | HRMS (HESI/FT) m/z: [M + 3H]³⁺ Calcd for | m/z 1204.0 [M + 3H]³⁺, R_{T} = 11.61 min |
| | C171H261N40O46 3607.908; Found 1203.6500 | |
| 31 | ND | m/z 1182.3 [M + 3H]³⁺, R_{T} = 8.67 min |
| 32 | HRMS (HESI/FT) m/z: [M + 3H]³⁺ Calcd for | m/z 1187.2 [M + 3H]³⁺, R_{T} = 9.11 min |
| | C168H257FN39O45 3556.8769; Found 1186.6393 | |
| 33 | ND | m/z 1177.3 [M + 3H]³⁺, R_{T} = 9.80 min |
| 34 | HRMS (HESI/FT) m/z: [M + 3H]³⁺ Calcd for | m/z 1061.8 [M + 3H]³⁺, R_{T} = 9.16 min |
| | C202H315FN41O57 4243.2759; Found 1415.7751 | |
| 35 | HRMS (HESI/FT) m/z: [M + 3H]³⁺ Calcd for | m/z 1065.9 [M + 3H]³⁺, R_{T} = 7.76 min |
| | C202H316FN42O57 4258.2871; Found 1420.4430 | |
| 36 | HRMS (HESI/FT) m/z: [M + 4H]⁴⁺ Calcd for | m/z 1176.4 [M + 3H]³⁺, R_{T} = 11.98 min |
| | C167H257N39O45 3524.8708; Found 882.2315 | |
| 37 | HRMS (HESI/FT) m/z: [M + 4H]⁴⁺ Calcd for | m/z 1180.4 [M + 3H]³⁺, R_{T} = 11.14 min |
| | C168H257N39O45 3536.8708; Found 885.2318 | |
| 38 | HRMS (HESI/FT) m/z: [M + 3H]³⁺ Calcd for | m/z 948.0 [M + 4H]⁴⁺, R_{T} = 14.16 min |
| | C178H270N45O47 3786.9885; Found 1263.3406 | |
| 39 | HRMS (HESI/FT) m/z: [M + 3H]³⁺ Calcd for | m/z 959.6 [M + 4H]⁴⁺, R_{T} = 12.75 min |
| | C182H280N43O48 3833.0557; Found 1278.6957 | |
| 40 | HRMS (HESI/FT) m/z: [M + 3H]³⁺ Calcd for | m/z 954.6 [M + 4H]⁴⁺, R_{T} = 12.96 min |
| | C180H275N46O46 3814.0359; Found 1272.3564 | |
| 41 | HRMS (HESI/FT) m/z: [M + 3H]³⁺ Calcd for | m/z 961.7 [M + 4H]⁴⁺, R_{T} = 10.45 min |
| | C182H279N46O46 3842.0671; Found 1281.6994 | |
| 42 | HRMS (HESI/FT) m/z: [M + 3H]³⁺ Calcd for | m/z 958.1 [M + 4H]⁴⁺, R_{T} = 12.79 min |
| | C181H277N46O46 3828.0515; Found 1277.0282 | |
| 43 | HRMS (HESI/FT) m/z: [M + 3H]³⁺ Calcd for | m/z 962.3 [M + 4H]⁴⁺, R_{T} = 10.98 min |
| | C183H280FN46O44 3842.0835; Found 1281.7068 | |
| 44 | HRMS (HESI/FT) m/z: [M + 3H]³⁺ Calcd for | m/z 969.2 [M + 4H]⁴⁺, R_{T} = 13.16 min |
| | C181H276N45O48 3845.0305; Found 1282.6891 | |
| 45 | HRMS (HESI/FT) m/z: [M + 3H]³⁺ Calcd for | m/z 1289.0 [M + 3H]³⁺, R_{T} = 12.02 min |
| | C183H281N46O47 3872.0776; Found 1291.7036 | |
| 46 | HRMS (HESI/FT) m/z: [M + 3H]³⁺ Calcd for | m/z 955.0 [M + 3H]³⁺, R_{T} = 8.38 min |
| | C183H282N43O49 3863.0662; Found 1288.6997 | |
| 47 | HRMS (HESI/FT) m/z: [M + 3H]³⁺ Calcd for | m/z 955.8 [M + 3H]³⁺, R_{T} = 8.34 min |
| | C181H278N43O48 3819.04; Found 1274.0220 | |
| 48 | HRMS (HESI/FT) m/z: [M + 3H]³⁺ Calcd for | m/z 960.4 [M + 4H]⁴⁺, R_{T} = 12.97 min |
| | C180H272FN42O50 3837.9783; Found 1280.3376 | |
| 49 | HRMS (HESI/FT) m/z: [M + 3H]³⁺ Calcd for | m/z 960.4 [M + 4H]⁴⁺, R_{T} = 13.48 min |
| | C181H277FN43O48 3837.0305; Found 1280.0217 | |
| 50 | HRMS (HESI/FT) m/z: [M + 3H]³⁺ Calcd for | m/z 963.6 [M + 4H]⁴⁺, R_{T} = 13.76 min |
| | C183H280FN42O48 3850.051; Found 1284.3613 | |
| 51 | HRMS (HESI/FT) m/z: [M + 3H]³⁺ Calcd for | m/z 960.1 [M + 4H]⁴⁺, R_{T} = 13.48 min |
| | C182H278FN42O48 3836.0354; Found 1279.6878 | |
| 52 | HRMS (HESI/FT) m/z: [M + 3H]³⁺ Calcd for | m/z 963.2 [M + 4H]⁴⁺, R_{T} = 11.32 min |
| | C180H271FN45O48 3846.9898; Found 1283.3414 | |
| 53 | HRMS (HESI/FT) m/z: [M + 3H]³⁺ Calcd for | m/z 962.7 [M + 4H]⁴⁺, R_{T} = 11.34 min |
| | C181H276FN46O46 3846.042; Found 1283.0241 | |
| 54 | HRMS (HESI/FT) m/z: [M + 3H]³⁺ Calcd for | m/z 1279.6 [M + 3H]³⁺, R_{T} = 14.11 min |
| | C182H279FN43O47 3835.0513; Found 1279.3650 | |
| 55 | HRMS (HESI/FT) m/z: [M + 3H]³⁺ Calcd for | m/z 1283.4 [M + 3H]³⁺, R_{T} = 14.31 min |
| | C181H276FN46O46 3846.042; Found 1283.0283 | |
| 56 | ND | m/z 1288.4 [M + 3H]³⁺, R_{T} = 14.70 min |
| 57 | HRMS (HESI/FT) m/z: [M + 3H]³⁺ Calcd for | m/z 1145.1 [M + 4H]⁴⁺, R_{T} = 14.45 min |
| | C217H339FN49O58 4575.4834; Found 1526.1749 | |
| 58 | HRMS (HESI/FT) m/z: [M + 4H]⁴⁺ Calcd for | m/z 1139.3 [M + 4H]⁴⁺, R_{T} = 14.32 min |
| | C217H340FN45O60 4551.4609; Found 1138.8811 | |
| 59 | HRMS (HESI/FT) m/z: [M + 3H]³⁺ Calcd for | m/z 1280.6 [M + 3H]³⁺, R_{T} = 9.90 min |
| | C181H277FN43O48 3837.0305; Found 1280.0261 | |
| 60 | ND | m/z 1285.2 [M + 3H]³⁺, R_{T} = 13.61 min |
| 61 | HRMS (HESI/FT) m/z: [M + 3H]³⁺ Calcd for | m/z 1139.7 [M + 4H]⁴⁺, R_{T} = 9.27 min |
| | C216H338FN46O60 4552.4561; Found 1518.4976 | |
| 62 | HRMS (HESI/FT) m/z: [M + 3H]³⁺ Calcd for | m/z 1142.7 [M + 4H]⁴⁺, R_{T} = 9.19 min |
| | C217H340FN46O60 4566.4717; Found 1523.1701 | |
| 63 | ND | m/z 1293.3 [M + 3H]³⁺, R_{T} = 9.34 min |
| 64 | HRMS (HESI/FT) m/z: [M + 3H]³⁺ Calcd for | m/z 1299.6 [M + 3H]³⁺, R_{T} = 9.60 min |
| | C184H283FN43O49 3895.0725; Found 1299.3680 | |
| 65 | HRMS (HESI/FT) m/z: [M + 3H]³⁺ Calcd for | m/z 1294.9 [M + 3H]³⁺, R_{T} = 10.68 min |
| | C184H282FN42O49 3880.0615; Found 1294.3663 | |
| 66 | HRMS (HESI/FT) m/z: [M + 4H]⁴⁺ Calcd for | m/z 1150.0 [M + 4H]⁴⁺, R_{T} = 13.74 min |
| | C219H344FN45O61 4595.4868; Found 1149.8902 | |
| 67 | HRMS (HESI/FT) m/z: [M + 4H]⁴⁺ Calcd for | m/z 1154.1 [M + 4H]⁴⁺, R_{T} = 12.37 min |
| | C219H345FN46O61 4610.4981; Found 1153.8913 | |
| 68 | HRMS (HESI/FT) m/z: [M + 3H]³⁺ Calcd for | m/z 1283.8 [M + 3H]³⁺, R_{T} = 10.43 min |
| | C183H281N42O49 3848.0552; Found 1283.6969 | |
| 69 | HRMS (HESI/FT) m/z: [M + 3H]³⁺ Calcd for | m/z 1288.4 [M + 3H]³⁺, R_{T} = 10.56 min |
| | C184H284N43O48 3861.0869; Found 1288.0379 | |
| 70 | HRMS (HESI/FT) m/z: [M + 3H]³⁺ Calcd for | m/z 974.8 [M + 4H]⁴⁺, R_{T} = 8.08 min |
| | C185H285FN43O48 3893.0933; Found 1299.0389 | |
| 71 | HRMS (HESI/FT) m/z: [M + 3H]³⁺ Calcd for | m/z 1303.1 [M + 3H]³⁺, R_{T} = 10.69 min |
| | C184H282FN46O47 3904.084; Found 1302.3744 | |
| 72 | HRMS (HESI/FT) m/z: [M + 4H]⁴⁺ Calcd for | m/z 1156.2 [M + 4H]⁴⁺, R_{T} = 13.85 min |
| | C219H344FN49O59 4619.5093; Found 1155.8940 | |
| 73 | HRMS (HESI/FT) m/z: [M + 4H]⁴⁺ Calcd for | m/z 1148.2 [M + 4H]⁴⁺, R_{T} = 13.57 min |
| | C218H343N49O59 4587.5034; Found 1147.8912 | |
| 74 | HRMS (HESI/FT) m/z: [M + 3H]³⁺ Calcd for | m/z 969.3 [M + 4H]⁴⁺, R_{T} = 13.57 min |
| | C184H280N43O49 3873.0505; Found 1292.0286 | |
| 75 | HRMS (HESI/FT) m/z: [M + 3H]³⁺ Calcd for | m/z 969.2 [M + 4H]⁴⁺, R_{T} = 10.03 min |
| | C185H285N44O47 3872.1028; Found 1291.7112 | |
| 76 | HRMS (HESI/FT) m/z: [M + 3H]³⁺ Calcd for | m/z 954.0 [M + 4H]⁴⁺, R_{T} = 12.31 min |
| | C178H274N43O50 3810.9985; Found 1271.3434 | |
| 77 | HRMS (HESI/FT) m/z: [M + 3H]³⁺ Calcd for | m/z 1271.3 [M + 3H]³⁺, R_{T} = 11.94 min |
| | C179H279N44O48 3810.0508; Found 1271.0264 | |
| 78 | HRMS (HESI/FT) m/z: [M + 3H]³⁺ Calcd for | m/z 971.6 [M + 4H]⁴⁺, R_{T} = 11.27 min |
| | C184H279N46O47 3882.062; Found 1295.0324 | |
| 79 | HRMS (HESI/FT) m/z: [M + 3H]³⁺ Calcd for | m/z 971.5 [M + 4H]⁴⁺, R_{T} = 14.49 min |
| | C185H284N47O45 3881.1145; Found 1294.7159 | |
| 80 | HRMS (HESI/FT) m/z: [M + 3H]³⁺ Calcd for | m/z 956.0 [M + 4H]⁴⁺, R_{T} = 13.70 min |
| | C178H273N46O48 3820.01; Found 1274.3477 | |
| 81 | HRMS (HESI/FT) m/z: [M + 3H]³⁺ Calcd for | m/z 955.9 [M + 4H]⁴⁺, R_{T} = 9.57 min |
| | C179H278N47O46 3819.0625; Found 1274.0306 | |

ND—Not Determined

### Synthesis of Examples 82-117

Standard Fmoc solid phase peptide synthesis (SPPS) was used to synthesize the peptides which were then cleaved from the resin and purified.

### General Method for Peptide Synthesis:

The peptide was synthesized using standard Fmoc chemistry.

### Method a—Exemplified by the Synthesis of Example 82

### Peptide Synthesis

1) Add DCM to the vessel containing Rink Amide MBHA Resin (sub: 0.35 mmol/g, 0.2 mmol, 0.57 g) and swell for 2 hours.
2) Drain and then wash with DMF (5 times, drain between each wash).
3) A solution of 20% piperidine in DMF was added agitate with N₂ bubbling for 30 min.
4) Drain and wash with DMF (5 times, drain between each wash).
5) Add Fmoc-amino acid solution (3.0 equivalents in DMF) and mix for 30 seconds, then add activation buffer (HBTU (2.85 equivalents) and DIEA (6 equivalents) in DMF), agitate with N₂ bubbling for 1 hour.
6) The coupling reaction was monitored by ninhydrin test
7) If required repeat steps 4 to 6 for same amino acid coupling if inefficient coupling occurs
8) Repeat steps 2 to 6 for next amino acid coupling.
   Note: for the acids in the table below different protecting groups and / or coupling agents were used

| Step | Materials | Coupling reagents |
|---|---|---|
| 22 | Fmoc-Asp(OAll)-OH (2.0 eq) | HATU (1.9 eq) and DIEA |
| | | (4.0 eq) |
| 26 | Fmoc-Lys(Alloc)-OH (3.0 eq) | HBTU (2.85 eq) and DIEA |
| | | (6.0 eq) |
| 31 | Intermediate 2 (2.0 eq) | DIC (2.0 eq) HOBT (2.0 eq) |
| 32 | Intermediate 1(1.5 eq) | DIC (1.5 eq) HOBT (1.5 eq) |

### Peptide Sidechain Deprotection Cyclisation:

1) Add DCM to the resin and agitate with N₂ bubbling, then add PhSiH₃ (10 eq), Pd(PPh₃)₄ (0.2 eq) agitate with N₂ for 15 mins for 3 times.
2) The resin was washed with DCM three times and then DMF three times.
3) The resin was washed with 0.5% Sodium diethyldithiocarbamate trihydrate DMF and 0.5% DIEA in DMF for ten times.
4) HATU (2 eq) and DIEA (4 eq) were added to the resin in DMF and agitate with N₂ bubbling for 1 hour.
5) The resin was washed with MeOH three times and dried in vacuo. Peptide Cleavage and Purification:
1) Add cleavage buffer (92.5% TFA/2.5% EDT/2.5% TIS/2.5% H₂O) to the flask containing the side chain protected peptide on resin at room temperature and stir for 3 hours.
2) Filter and collect the peptide solution.
3) The peptide is precipitated with cold tert-butyl methyl ether and centrifuged (3 min at 3000 rpm).
4) Residue washed with tert-butyl methyl ether (2 times).
5) Crude peptide dried under vacuum for 2 hours.
6) The crude peptide was purified by prep-HPLC. Prep-HPLC Conditions: Instrument: Gilson 281. Solvent: A- 0.1% TFA in H2O, B—acetonitrile, Column: Luna C18 (200×25 mm; 10 μm) and Gemini C18 (150*30 mm; 5 μm) in series. Gradient [time (min)/solvent B (%)]:0.0/25, 60.0/55, 60.1/90, 70/90, 70.1/10, at 20 mL/min with UV detection (wave length=215/254 nm) and then lyophilized to give Example 1 (10.3 mg, 1.34% yield).

### Method b—Exemplified by the Synthesis of Example 105

### Peptide Synthesis

9) Add DCM to the vessel containing Rink Amide MBHA Resin (sub: 0.35 mmol/g, 0.15 mmol, 0.42 g) and swell for 2 hours.
10) Drain and then wash with DMF (5 times, drain between each wash).
11) A solution of 20% piperidine in DMF was added agitate with N₂ bubbling for 30 min.
12) Drain and wash with DMF (5 times, drain between each wash).
13) Add Fmoc-amino acid solution (3.0 equivalents in DMF) and mix for 30 seconds, then add activation buffer (HBTU (2.85 equivalents) and DIEA (6 equivalents) in DMF), agitate with N₂ bubbling for 1 hour.
14) The coupling reaction was monitored by ninhydrin test
15) If required repeat steps 4 to 6 for same amino acid coupling if inefficient coupling occurs
16) Repeat steps 2 to 6 for next amino acid coupling.
   Note: for the acids in the table below different protecting groups and / or coupling agents were used.

| Step | Materials | Coupling reagents |
|---|---|---|
| 1 | Intermediate 5 (2.0 eq) | DIC (2.0 eq) and HOBT (2.0 eq) |
| 16 | Intermediate 8 (2.0 eq) | DIC (2.0 eq) and HOBT (2.0 eq) |
| 25 | Intermediate 2 (2.0 eq) | DIC (2.0 eq) HOBT (2.0 eq) |
| 26 | Intermediate 1(1.5 eq) | DIC (1.5 eq) HOBT (1.5 eq) |

10) The resin was washed with DMF five times and MeOH three times and dried in vacuo.

### Peptide Cleavage and Purification:

7) Add cleavage buffer (92.5% TFA/2.5% EDT/2.5% TIS/2.5% H₂O) to the flask containing the side chain protected peptide on resin at room temperature and stir for 3 hours.
8) Filter and collect the peptide solution.
9) The peptide is precipitated with cold tert-butyl methyl ether and centrifuged (3 min at 3000 rpm).
10) Residue washed with tert-butyl methyl ether (2 times).
11) Crude peptide dried under vacuum for 2 hours.
12) The crude peptide was purified by prep-HPLC. Prep-HPLC Conditions: Instrument: Gilson 281. Solvent: A- 0.1% TFA in H2O, B—acetonitrile, Column: Luna C18 (200×25 mm; 10 μm) and Gemini C18 (150*30 mm; 5 μm) in series. Gradient [time (min)/solvent B (%)]:0.0/25, 60.0/55, 60.1/90, 70/90, 70.1/10, at 20 mL/min with UV detection (wave length=215/254 nm) and then lyophilized to give Example 24 (109.8 mg, 19.3% yield).

TABLE 2a
HRMS and LCMS properties of purified peptides represented by Examples 82-117

| | Synthetic | HRMS | LCMS/HPLC |
|---|---|---|---|
| Example | Method | (Analytical Method D) | (Analytical Method C) |
| 82 | a | HRMS (HESI/FT) m/z: [M + 4H]⁴⁺ Calcd for | m/z 958.2 [M + 4H]⁴⁺, R_{T} = 10.69 min |
| | | C181H278N46O46 3828.0515; Found | |
| | | 958.0243 | |
| 83 | a | HRMS (HESI/FT) m/z: [M + 3H]³⁺ Calcd for | m/z 946.8 [M + 4H]⁴⁺, R_{T} = 12.19 min |
| | | C175H270N45O49 3782.9785; Found | |
| | | 1262.0046 | |
| 84 | a | HRMS (HESI/FT) m/z: [M + 4H]⁴⁺ Calcd for | m/z 959.0 [M + 4H]⁴⁺, R_{T} = 11.33 min |
| | | C178H279N43O51 3831.0247; Found | |
| | | 958.7668 | |
| 85 | a | HRMS (HESI/FT) m/z: [M + 4H]⁴⁺ Calcd for | m/z 947.9 [M + 4H]⁴⁺, R_{T} = 11.65 min |
| | | C176H275N43O50 3786.9985; Found | |
| | | 947.7603 | |
| 86 | a | HRMS (HESI/FT) m/z: [M + 4H]⁴⁺ Calcd for | m/z 1248.4 [M + 3H]³⁺, R_{T} = 10.54 min |
| | | C171H273N43O51 3740.9778; Found | |
| | | 936.2551 | |
| 87 | a | HRMS (HESI/FT) m/z: [M + 4H]⁴⁺ Calcd for | m/z 952.1 [M + 4H]⁴⁺, R_{T} = 12.91 min |
| | | C175H271N43O52 3802.957; Found | |
| | | 951.7492 | |
| 88 | a | HRMS (HESI/FT) m/z: [M + 4H]⁴⁺ Calcd for | m/z 948.5 [M + 4H]⁴⁺, R_{T} = 9.69 min |
| | | C174H269N43O52 3788.9414; Found | |
| | | 948.2462 | |
| 89 | a | HRMS (HESI/FT) m/z: [M + 4H]⁴⁺ Calcd for | m/z 945.2 [M + 4H]⁴⁺, R_{T} = 10.60 min |
| | | C173H266N42O53 3775.9097; Found | |
| | | 944.9882 | |
| 90 | a | HRMS (HESI/FT) m/z: [M + 4H]⁴⁺ Calcd for | m/z 944.5 [M + 4H]⁴⁺, R_{T} = 14.42 min |
| | | C175H272N42O51 3775.9097; Found | |
| | | 944.5023 | |
| 91 | a | HRMS (HESI/FT) m/z: [M + 4H]⁴⁺ Calcd for | m/z 1278.0 [M + 3H]³⁺, R_{T} = 12.20 min |
| | | C179H272N46O48 3829.9944; Found | |
| | | 958.5097 | |
| 92 | a | HRMS (HESI/FT) m/z: [M + 4H]⁴⁺ Calcd for | m/z 941.8 [M + 4H]⁴⁺, R_{T} = 11.15 min |
| | | C172H264N42O53 3761.894; Found | |
| | | 941.4835 | |
| 93 | a | HRMS (HESI/FT) m/z: [M + 4H]⁴⁺ Calcd for | m/z 951.7 [M + 4H]⁴⁺, R_{T} = 10.72 min |
| | | C177H277N43O50 3801.0142; Found | |
| | | 951.2633 | |
| 94 | a | HRMS (HESI/FT) m/z: [M + 4H]⁴⁺ Calcd for | m/z 1262.4 [M + 3H]³⁺, R_{T} = 10.33 min |
| | | C173H275N43O52 3782.9883; Found | |
| | | 946.7572 | |
| 95 | a | HRMS (HESI/FT) m/z: [M + 3H]³⁺ Calcd for | m/z 1269.5 [M + 3H]³⁺, R_{T} = 13.26 min |
| | | C177H266N45O49 3802.947; Found | |
| | | 1268.6602 | |
| 96 | a | HRMS (HESI/FT) m/z: [M + 3H]³⁺ Calcd for | m/z 1273.7 [M + 3H]³⁺, R_{T} = 10.38 min |
| | | C178H268N45O49 3816.9629; Found | |
| | | 1273.3316 | |
| 97 | a | HRMS (HESI/FT) m/z: [M + 4H]⁴⁺ Calcd for | m/z 1264.3 [M + 3H]³⁺, R_{T} = 12.93 min |
| | | C174H269N43O52 3788.9414; Found | |
| | | 948.2459 | |
| 98 | a | HRMS (HESI/FT) m/z: [M + 3H]³⁺ Calcd for | m/z 1254.8 [M + 3H]³⁺, R_{T} = 11.56 min |
| | | C173H264N41O53 3760.8989; Found | |
| | | 1254.6640 | |
| 99 | a | HRMS (HESI/FT) m/z: [M + 4H]⁴⁺ Calcd for | m/z 1259.6 [M + 3H]³⁺, R_{T} = 14.37 min |
| | | C176H275N41O51 3774.9873; Found | |
| | | 944.7565 | |
| 100 | a | HRMS (HESI/FT) m/z: [M + 4H]⁴⁺ Calcd for | m/z 1254.9 [M + 3H]³⁺, R_{T} = 11.68 min |
| | | C175H273N41O51 3760.9717; Found | |
| | | 941.2530 | |
| 101 | a | HRMS (HESI/FT) m/z: [M + 4H]⁴⁺ Calcd for | m/z 1259.2 [M + 3H]³⁺, R_{T} = 13.27 min |
| | | C175H272N42O51 3773.9668; Found | |
| | | 944.5030 | |
| 102 | a | ND | m/z 1260.0 [M + 3H]³⁺, R_{T} = 14.58 min |
| 103 | a | HRMS (HESI/FT) m/z: [M + 3H]³⁺ Calcd for | m/z 1255.3 [M + 3H]³⁺, R_{T} = 14.15 min |
| | | C172H263N42O53 3761.894; Found | |
| | | 1253.9754 | |
| 104 | b | HRMS (HESI/FT) m/z: [M + 4H]⁴⁺ Calcd for | m/z 961.7 [M + 4H]⁴⁺, R_{T} = 12.18 min |
| | | C180H281N43O50 3841.0454; Found | |
| | | 961.2712 | |
| 105 | b | HRMS (HESI/FT) m/z: [M + 3H]³⁺ Calcd for | m/z 1266.1 [M + 3H]³⁺, R_{T} = 13.55 min |
| | | C175H267N46O49 3793.958; Found | |
| | | 1265.6650 | |
| 106 | b | HRMS (HESI/FT) m/z: [M + 3H]³⁺ Calcd for | m/z 1256.9 [M + 3H]³⁺, R_{T} = 12.15 min |
| | | C173H262N45O50 3766.9106; Found | |
| | | 1256.6490 | |
| 107 | b | HRMS (HESI/FT) m/z: [M + 3H]³⁺ Calcd for | m/z 1261.7 [M + 3H]³⁺, R_{T} = 15.55 min |
| | | C174H264N45O50 3780.9265; Found | |
| | | 1261.3203 | |
| 108 | b | HRMS (HESI/FT) m/z: [M + 3H]³⁺ Calcd for | m/z 1251.2 [M + 3H]³⁺, R_{T} = 13.59 min |
| | | C175H268N45O47 3748.9729; Found | |
| | | 1250.6702 | |
| 109 | b | HRMS (HESI/FT) m/z: [M + 3H]³⁺ Calcd for | m/z 1251.6 [M + 3H]³⁺, R_{T} = 12.52 min |
| | | C173H262N45O49 3750.9158; Found | |
| | | 1251.3169 | |
| 110 | b | HRMS (HESI/FT) m/z: [M + 4H]⁴⁺ Calcd for | m/z 1277.9 [M + 3H]³⁺, R_{T} = 8.06 min |
| | | C177H273N43O52 3828.9727; Found | |
| | | 958.2533 | |
| 111 | b | HRMS (HESI/FT) m/z: [M + 4H]⁴⁺ Calcd for | m/z 1261.0 [M + 3H]³⁺, R_{T} = 10.42 min |
| | | C174H275N43O51 3778.9934; Found | |
| | | 945.7591 | |
| 112 | b | HRMS (HESI/FT) m/z: [M + 4H]⁴⁺ Calcd for | m/z 1256.2 [M + 3H]³⁺, R_{T} = 13.70 min |
| | | C174H278N44O49 3764.03; Found | |
| | | 942.0182 | |
| 113 | b | HRMS (HESI/FT) m/z: [M + 4H]⁴⁺ Calcd for | m/z 1267.6 [M + 3H]³⁺, R_{T} = 14.43 min |
| | | C176H271N43O51 3798.9622; Found | |
| | | 950.7509 | |
| 114 | b | HRMS (HESI/FT) m/z: [M + 3H]³⁺ Calcd for | m/z 1272.2 [M + 3H]³⁺, R_{T} = 10.81 min |
| | | C179H269N46O47 3811.9839; Found | |
| | | 1271.6720 | |
| 115 | b | HRMS (HESI/FT) m/z: [M + 3H]³⁺ Calcd for | m/z 1277.1 [M + 3H]³⁺, R_{T} = 10.95 min |
| | | C180H271N46O47 3825.9995; Found | |
| | | 1276.3429 | |
| 116 | b | HRMS (HESI/FT) m/z: [M + 3H]³⁺ Calcd for | m/z 1282.0 [M + 3H]³⁺, R_{T} = 11.25 min |
| | | C182H275N44O48 3842.0195; Found | |
| | | 1281.6846 | |
| 117 | b | HRMS (HESI/FT) m/z: [M + 3H]³⁺ Calcd for | m/z 1161.3 [M + 3H]³⁺, R_{T} = 12.13 min |
| | | C163H248N41O44 3480.8193; Found | |
| | | 1161.2830 | |

ND—Not determined

### Biological Activity

The following examples are provided to illustrate preferred aspects of the invention and are not intended to limit the scope of the invention.

### Example A. In Vitro Pharmacological Characterization of Peptides—Functional Agonism of Human GLP2 or GLP1 Receptors, cAMP Accumulation Assay

CAMP production upon agonist stimulation of human GLP2 or GLP1 receptor was assessed utilizing HiRange cAMP kit (Cisbio). In brief, HEK cells were infected with either human GLP2 or GLP1 receptor BacMam virus for 24 hours and frozen for later use in the assay. On the day, various concentrations of compounds were dispensed using ECHO-555 (LabCyte) to a total volume of 100 nl into a low volume 384-well Proxi plates (Perkin Elmer) followed by addition of 10 μl of cell suspension delivering 800 k cells per well. Cells were prepared in the assay buffer (HBSS (Lonza) supplemented with 0.5 mM IBMX (Tocris)). After 45 min incubation at 37° C., the reaction was stopped by addition of the HTRF detection reagents in the lysis buffer provided in the kit. Following 1-hour incubation at RT, plates were read on Pherastar FS (BMG Labtech, Inc.) Dotmatics Studies software was used for calculation of pEC₅₀ values by fitting data to a four parameter dose response curve.

Exendin-4 and liraglutide were used as reference compounds for GLP-1 receptor activation whilst Teduglutide and FE-203799 were used as reference compounds for GLP-2 receptor activation.

| | |
|---|---|
| Human GLP-1R | Human GLP-2R |
| agonist cAMP | agonist cAMP |

| Example | pEC₅₀ | Emax | pEC₅₀ | Emax |
|---|---|---|---|---|
| Teduglutide | 6.5 | 60.3 | 11.8 | 100.6 |
| FE-203799 | 6.8 | 61.4 | 10.8 | 100.8 |
| Exendin-4 | 12.4 | 99.7 | <8.1 | 1.6 |
| liraglutide | 11.5 | 97 | <6.1 | 1 |
| 1 | 9.1 | 90.1 | 9.4 | 103.1 |
| 2 | 9.4 | 92.5 | 10.5 | 103.2 |
| 3 | 9.7 | 94.7 | 9.2 | 95.4 |
| 4 | 9.3 | 94.7 | 11.0 | 103.4 |
| 5 | 9.7 | 92.5 | 9.9 | 103.7 |
| 6 | 7.1 | 91.3 | 9.3 | 102.7 |
| 7 | 7.8 | 72.7 | 9.4 | 103.3 |
| 8 | 9.3 | 90.8 | 9.9 | 102.9 |
| 9 | 8.2 | 62.5 | 10.0 | 104.0 |
| 10 | 8.5 | 60.4 | 10.2 | 105.1 |
| 11 | 9.6 | 95.3 | 10.1 | 105.0 |
| 12 | 9.6 | 94.5 | 10.3 | 105.3 |
| 13 | 9.6 | 92.5 | 10.5 | 104.1 |
| 14 | 9.4 | 93.0 | 10.5 | 103.8 |
| 15 | 10.1 | 98.2 | 10.8 | 103.5 |
| 16 | 9.2 | 94.1 | 9.6 | 103.8 |
| 17 | 9.8 | 96.3 | 10.4 | 103.8 |
| 18 | 10.0 | 97.8 | 10.8 | 104.2 |
| 19 | 9.6 | 95.4 | 11.0 | 103.4 |
| 20 | 8.8 | 84.1 | 10.3 | 104.2 |
| 21 | 10.3 | 96.6 | 10.8 | 104.1 |
| 22 | 9.4 | 92.7 | 11.1 | 103.7 |
| 23 | 9.1 | 94.3 | 10.6 | 102.2 |
| 24 | 9.7 | 96.8 | 11.1 | 102.6 |
| 25 | 10.5 | 97.6 | 10.6 | 103.0 |
| 26 | 9.8 | 97.1 | 10.8 | 102.9 |
| 27 | 9.6 | 97.1 | 10.2 | 99.3 |
| 28 | 10.1 | 96.0 | 9.5 | 99.6 |
| 29 | 10.3 | 96.2 | 10.5 | 103.1 |
| 30 | 10.3 | 96.7 | 10.6 | 103.7 |
| 31 | 10.8 | 98.3 | 10.4 | 103.4 |
| 32 | 10.6 | 97.8 | 9.1 | 91.3 |
| 33 | 10.6 | 98.1 | 10.5 | 102.5 |
| 34 | 8.9 | 88.0 | 9.7 | 101.7 |
| 35 | 9.1 | 94.5 | 9.7 | 102.8 |
| 36 | 10.5 | 97.1 | 10.5 | 103.7 |
| 37 | 10.4 | 97.0 | 10.6 | 103.4 |
| 38 | 9.3 | 88.8 | 10.9 | 104.9 |
| 39 | 9.4 | 92.6 | 9.7 | 105.2 |
| 40 | 9.3 | 89.9 | 11.2 | 105.3 |
| 41 | 9.6 | 95.3 | 9.1 | 97.6 |
| 42 | 9.7 | 94.1 | 9.4 | 96.4 |
| 43 | 9.2 | 95.2 | 9.4 | 104.8 |
| 44 | 9.7 | 96.1 | 9.2 | 103.5 |
| 45 | 9.7 | 96.5 | 10.3 | 103.6 |
| 46 | 9.9 | 97.8 | 10.6 | 104.8 |
| 47 | 9.5 | 93.6 | 9.8 | 104.4 |
| 48 | 10.8 | 98.7 | 11.1 | 101.3 |
| 49 | 10.9 | 98.9 | 10.9 | 102.1 |
| 50 | 10.0 | 98.3 | 10.6 | 102.3 |
| 51 | 9.9 | 97.8 | 10.9 | 102.9 |
| 52 | 11.6 | 98.8 | 11.0 | 101.5 |
| 53 | 11.3 | 99.5 | 10.7 | 101.6 |
| 54 | 9.2 | 88.8 | 10.5 | 101.4 |
| 55 | 10.3 | 97.9 | 11.0 | 103.1 |
| 56 | 10.2 | 97.3 | 10.9 | 102.6 |
| 57 | 8.9 | 91.6 | 10.5 | 102.4 |
| 58 | 8.3 | 62.8 | 10.2 | 101.8 |
| 59 | 9.7 | 95.2 | 10.7 | 103.1 |
| 60 | 9.8 | 96.5 | 10.8 | 102.4 |
| 61 | 8.5 | 69.1 | 10.5 | 101.4 |
| 62 | 8.4 | 71.5 | 10.4 | 102.6 |
| 63 | 9.0 | 96.9 | 10.0 | 99.7 |
| 64 | 10.6 | 98.0 | 10.4 | 103.6 |
| 65 | 10.4 | 97.9 | 10.4 | 102.1 |
| 66 | 8.8 | 88.1 | 9.6 | 101.0 |
| 67 | 9.1 | 95.1 | 9.8 | 103.0 |
| 68 | 9.5 | 94.0 | 10.1 | 103.1 |
| 69 | 8.2 | 83.7 | 9.3 | 95.0 |
| 70 | 10.4 | 98.2 | 9.4 | 97.4 |
| 71 | 10.6 | 98.2 | 10.6 | 102.6 |
| 72 | 9.5 | 95.7 | 9.5 | 100.9 |
| 73 | 8.7 | 87.2 | 9.3 | 100.0 |
| 74 | 9.2 | 90.9 | 10.8 | 101.6 |
| 75 | 9.2 | 95.0 | 10.4 | 102.3 |
| 76 | 9.0 | 94.9 | 10.9 | 102.0 |
| 77 | 9.3 | 97.1 | 10.2 | 102.1 |
| 78 | 9.6 | 95.5 | 10.6 | 103.0 |
| 79 | 9.6 | 97.3 | 10.5 | 102.9 |
| 80 | 8.4 | 64.1 | 9.7 | 99.9 |
| 81 | 8.5 | 56.1 | 9.2 | 91.6 |
| 82 | 7.9 | 94.5 | 10.1 | 104.5 |
| 83 | 9.2 | 98.1 | 9.6 | 103.4 |
| 84 | 8.0 | 87.3 | 10.3 | 103.2 |
| 85 | 7.9 | 95.2 | 10.5 | 103.0 |
| 86 | 7.6 | 93.5 | 10.6 | 102.8 |
| 87 | 8.6 | 98.7 | 11.6 | 102.0 |
| 88 | 8.9 | 99.2 | 11.5 | 102.3 |
| 89 | 8.5 | 99.6 | 11.6 | 102.5 |
| 90 | 8.1 | 100.2 | 10.6 | 102.1 |
| 91 | 9.6 | 98.8 | 11.0 | 103.9 |
| 92 | 8.0 | 96.5 | 11.7 | 99.3 |
| 93 | 8.4 | 95.3 | 11.3 | 98.6 |
| 94 | 8.3 | 92.6 | 11.0 | 103.1 |
| 95 | 8.4 | 97.0 | 11.1 | 102.1 |
| 96 | 9.3 | 96.4 | 11.2 | 100.6 |
| 97 | 9.5 | 96.6 | 11.5 | 101.0 |
| 98 | 7.7 | 96.0 | 11.2 | 101.3 |
| 99 | 8.6 | 97.1 | 11.3 | 101.4 |
| 100 | 9.1 | 96.0 | 11.3 | 100.0 |
| 101 | 7.9 | 96.7 | 10.5 | 100.8 |
| 102 | 7.7 | 99.3 | 11.9 | 100.9 |
| 103 | 8.7 | 97.0 | 11.8 | 100.4 |
| 104 | 8.4 | 88.3 | 9.3 | 102.4 |
| 105 | 10.6 | 97.4 | 10.6 | 100.9 |
| 106 | 9.5 | 97.2 | 9.3 | 102.1 |
| 107 | 10.7 | 96.9 | 10.7 | 101.4 |
| 108 | 8.4 | 92.1 | 9.3 | 104.1 |
| 109 | 10.6 | 97.0 | 11.1 | 102.5 |
| 110 | 8.4 | 88.1 | 10.8 | 103.0 |
| 111 | 9.1 | 93.2 | 9.2 | 104.2 |
| 112 | 8.0 | 92.6 | 9.8 | 102.1 |
| 113 | 8.7 | 97.0 | 11.5 | 101.0 |
| 114 | 8.5 | 89.1 | 10.7 | 102.1 |
| 115 | 9.3 | 92.0 | 10.7 | 104.4 |
| 116 | 9.5 | 94.4 | 11.1 | 102.8 |
| 117 | 9.9 | 94.7 | 11.5 | 103.4 |

### Example B. In Vitro Pharmacological Characterization of Peptides—Functional Agonism of Mouse GLP2 or GLP1 Receptors, cAMP Accumulation Assay:

CAMP production upon agonist stimulation of mouse GLP2 or GLP1 receptors was assessed utilizing HiRange cAMP kit (Cisbio). In brief, HEK cells were transiently transfected for 24 hours with cDNA using GeneJuice Transfection reagent (EMD Millipore) and frozen at −80° C. for later use in the assay. On the day, various concentrations of compounds were dispensed using ECHO-555 (LabCyte) to a total volume of 100 nl into a low volume 384-well Proxi plate (Perkin Elmer) followed by addition of 10 μl of cell suspension delivering 8000 cells per well. Cells were prepared in the assay buffer (HBSS (Lonza) supplemented with 0.5 mM IBMX (Tocris)). After 45 min incubation at 37° C., the reaction was stopped by addition of the HTRF detection reagents in the lysis buffer provided in the kit. Following 1-hour incubation at RT, plates were read on Pherastar FS (BMG Labtech, Inc.) using standard HTRF settings. Dotmatics Studies software was used for calculation of pEC50 values by fitting data to a four-parameter concentration response curve.

Liraglutide was used as reference compound for GLP-1 receptor activation whilst Teduglutide and FE-203799 were used as reference compounds for GLP-2 receptor activation.

| | |
|---|---|
| Mouse GLP-1R | Mouse GLP-2R |
| agonist cAMP | agonist cAMP |

| Example | pEC₅₀ | Emax | pEC₅₀ | Emax |
|---|---|---|---|---|
| Teduglutide | <6.1 | 1 | 11.1 | 100.1 |
| FE-203799 | <7.3 | 53.4 | 10.5 | 98.1 |
| liraglutide | 11.4 | 97.9 | <6.1 | 1.6 |
| 1 | 9.9 | 91.5 | 8.0 | 97.7 |
| 2 | 10.4 | 94.3 | 9.0 | 91.6 |
| 19 | ND | ND | 9.1 | 100.6 |
| 22 | ND | ND | 8.8 | 94.5 |
| 26 | ND | ND | 8.5 | 86.4 |
| 29 | ND | ND | 8.3 | 100.0 |
| 30 | ND | ND | 8.9 | 101.3 |
| 31 | ND | ND | 9.8 | 101.9 |
| 38 | ND | ND | 8.5 | 99.0 |
| 40 | ND | ND | 8.9 | 99.6 |
| 46 | ND | ND | 8.0 | 100.8 |
| 48 | ND | ND | 10.2 | 102.9 |
| 49 | ND | ND | 9.6 | 101.74 |
| 50 | ND | ND | 8.1 | 81.0 |
| 51 | ND | ND | 8.4 | 87.8 |
| 52 | ND | ND | 9.7 | 103.1 |
| 53 | ND | ND | 9.2 | 103.1 |
| 55 | ND | ND | 9.5 | 100.6 |
| 56 | ND | ND | 9.2 | 98.6 |
| 59 | ND | ND | 9.3 | 102.7 |
| 60 | ND | ND | 9.1 | 102.5 |
| 64 | ND | ND | 8.7 | 99.7 |
| 65 | ND | ND | 8.2 | 86.3 |
| 71 | ND | ND | 8.7 | 89.71 |
| 74 | ND | ND | 8.8 | 93.6 |
| 75 | ND | ND | 8.2 | 81.9 |
| 76 | ND | ND | 8.9 | 102.5 |
| 77 | ND | ND | 8.5 | 102.2 |
| 78 | ND | ND | 8.7 | 83.1 |
| 79 | ND | ND | 8.5 | 74.2 |
| 87 | 9.2 | 91.4 | 11.2 | 99.2 |
| 88 | 10.0 | 96.4 | 11.2 | 98.7 |
| 89 | 9.1 | 94.5 | 11.2 | 98.4 |
| 92 | 8.0 | 94.8 | 10.9 | 98.9 |
| 93 | 8.6 | 95.5 | 9.6 | 98.5 |
| 95 | 8.3 | 94.7 | 11.1 | 102.1 |
| 96 | 9.1 | 96.1 | 9.1 | 100.7 |
| 102 | ND | ND | 10.7 | 99.0 |
| 112 | ND | ND | 8.2 | 99.7 |
| 113 | 8.8 | 96.3 | 9.7 | 99.2 |
| 116 | 9.7 | 95.5 | 9.5 | 99.9 |
| 117 | 10.1 | 96.9 | 9.8 | 99.3 |

ND—Not determined

### Example C: In Vitro Pharmacological Characterization of Peptides—Evaluation of the Stability of Peptides in Fasted state Simulated Intestinal fluid

Stability of peptides was tested in Fasted-State Simulated Intestinal Fluid (FaSSIF) prepared according to manufacturer's protocol (Biorelevant, art.no. FFF01, pH 6.5). FaSSIF composition: 3 mM sodium taurocholate, 0.75 mM lecithin, 105.9 mM NaCl, 28.4 mM Na₂HPO4, 8.7 mM NaOH, and 10 mg/ml pancreatin (Sigma). FaSSIF was pre-incubated for 15 min at 37° C. and spiked with test and reference item working solutions. Experiments were conducted in duplicate in a non-serial manner. The total incubation volume per replicate was 150 μl. Sampling time points for test items were 0, 0.5, 2, 5, 10, 15 and 30 min. All samples and calibration standards (prepared in FaSSIF) were precipitated by addition of 300 μl precipitant (ACN / 2% acetic acid / 0.2% HFBA, (precipitation reagent, PR)) containing internal standard (ISTD) to 150 μl sample. After incubation for 1 h at room temperature all samples were centrifuged for 10 min at 2,200×g (room temperature). Prior to subjection to LC-MS, the samples were diluted 1:1 in PBS buffer in order to reduce the organic solvent content in the samples to 33%.

The % of compound remaining at t=30 mins is summarised below. Neurotensin was included as a reference agent.

| | FASSIF % remaining |
|---|---|
| Example | (at t = 30) |
| Neurotensin | 0-2.7 |
| Teduglutide | 1.7 |
| 1 | 5 |
| 2 | 11 |
| 4 | 4.3 |
| 10 | 3.8 |
| 11 | 22.3 |
| 12 | 10.7 |
| 13 | 3.8 |
| 14 | 17.1 |
| 15 | 15.8 |
| 17 | 68.7 |
| 18 | 17.3 |
| 19 | 12.2 |
| 20 | 8.6 |
| 21 | 51.7 |
| 22 | 6.8 |
| 30 | 51.1 |
| 38 | 61.3 |
| 39 | 100 |
| 40 | 68.7 |
| 45 | 95.3 |
| 46 | 95.3 |
| 48 | 80.2 |
| 59 | 61.9 |
| 60 | 62.5 |

The % of compound remaining at t=15 mins is summarised below. Neurotensin was included as a reference agent.

| | % remaining at |
|---|---|
| | t = 15 mins |
| Example | (FassIF) |
| Neurotensin | 1.2 |
| Teduglutide | 1.2 |
| 86 | 5.1 |
| 87 | 1.7 |
| 88 | 0.5 |
| 89 | 10.5 |
| 90 | 22 |
| 91 | 8.8 |
| 93 | 13.5 |
| 105 | 1.6 |
| 106 | 9.6 |
| 107 | 7.4 |
| 109 | 4.6 |
| 110 | 5 |
| 111 | 26.9 |
| 112 | 40.7 |
| 113 | 3.4 |

### Example D: In Vitro Pharmacological Characterization of Peptides—Evaluation of the Stability of Peptides in Fasted State Simulated Gastric Fluid

Stability of peptides was tested in Fasted-State Simulated gastric Fluid (FaSSGF) prepared according to manufacturer's protocol (Biorelevant, art. no. FFF01). FaSSGF composition: 0.08 mM sodium taurocholate, 0.02 mM lecithin, 34.2 mM NaCl, 25.1 mM HCL, and 0.1 mg/ml pepsin (Sigma). pH was adjusted to 1.6. FaSSGF was pre-incubated for 15 min at 37° C. and spiked with test and reference item working solutions. Experiments were conducted in duplicate in a non-serial manner. The total incubation volume per replicate was 150 μl. Sampling time points for test items and reference item neurotensin were 0, 0.5, 2, 5, 10, 15 and 30 min. All samples and calibration standards (prepared in FaSSGF) were precipitated by addition of 300 μl precipitant (ACN / 2% acetic acid / 0.2% HFBA, (precipitation regent, PR)) containing internal standards (ISTD) to 150 μl sample. After incubation for 1 h at room temperature all samples were centrifuged for 10 min at 2,200×g (room temperature). Prior to subjection to LCMS, the samples were diluted 1:1 in PBS buffer in order to reduce the organic solvent content in the samples to 33%.

The % of compound remaining at t=30 mins is summarised below. Neurotensin was included as a reference agent.

| | FASSGF % remaining |
|---|---|
| Example | (at t = 30) |
| Neurotensin | 22 |
| Teduglutide | 9.2 |
| 2 | 34 |
| 30 | 100 |
| 38 | 100 |
| 39 | 100 |
| 40 | 84.9 |
| 45 | 100 |
| 46 | 100 |
| 48 | 100 |
| 59 | 100 |
| 60 | 100 |

### Example E: In Vitro Pharmacological Characterization of Peptides—Evaluation of the Stability of Peptides in Rat Intestinal Fluid

Peptides were tested for in vitro stability in native intestinal fluid obtained from the rat small intestine. Rat Sprague Dawley Small Intestinal Fluid (ratIF) (from Biotrend art. no. RSD-SIF-MI-30ML, undiluted) was preincubated for 15 min at 37° C. and spiked with test and reference item working solutions. Experiments were conducted in duplicate in a non-serial manner. The total incubation volume per replicate was 150 μl. Sampling time points for test items and reference item neurotensin were 0, 0.5, 2, 5, 10, 15 and 30 min. All samples and calibration standards (prepared in ratIF) were precipitated by addition of 300 μl precipitant (ACN / 2% acetic acid / 0.2% HFBA, (precipitation regent, PR)) containing internal standards (ISTD) to 150 μl sample. After incubation for 1 h at room temperature all samples were centrifuged for 10 min at 2,200×g (room temperature). The resulting samples were transferred to auto sampler vials and subsequently subjected to LC-MS analysis to subjection to LC-MS.

The % of compound remaining at t=30 mins is summarised below. Neurotensin was included as a reference agent.

| | Rat IF % remaining |
|---|---|
| Example | (at t = 30) |
| Neurotensin | 0.4 |
| Teduglutide | 1.3 |
| 2 | 22.5 |
| 27 | 11 |
| 29 | 41.3 |
| 31 | 24.8 |
| 36 | 23.3 |
| 37 | 26.8 |
| 38 | 33.4 |
| 39 | 12.7 |
| 40 | 14.7 |
| 45 | 62.4 |
| 48 | 100 |
| 51 | 66.9 |
| 55 | 64.9 |
| 57 | 79.9 |
| 59 | 63.6 |
| 60 | 89.6 |
| 71 | 73.8 |
| 72 | 82.8 |
| 74 | 71.2 |

The % of compound remaining at t=15 mins is summarised below. Neurotensin was included as a reference agent.

| | % remaining at |
|---|---|
| Example | t = 15 mins (ratIF) |
| Neurotensin | 0.1 |
| Teduglutide | 5.7 |
| 112 | 24.5 |

1. A compound comprising the sequence of formula (1a) or formula (1b): 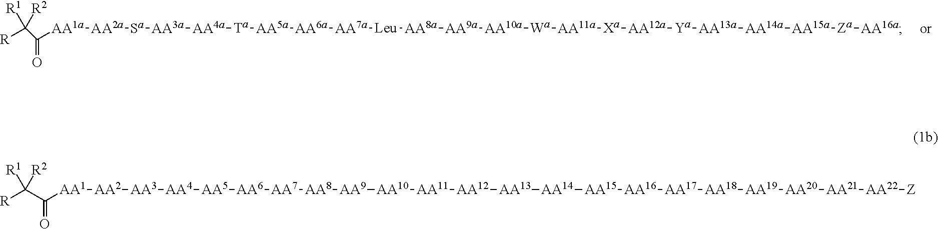
wherein;
R is selected from: 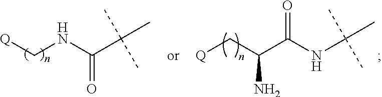
Q is phenyl or a monocyclic heteroaryl ring each of which may be optionally substituted with one or more R^{q} groups;
R^{q} is selected from halogen, hydroxyl, amino or C₁₋₆ alkyl having an alkyl chain optionally containing one or more heteroatoms selected from O, N, or S;
n is 1 to 3;
R¹ and R² are independently selected from hydrogen or a C₁₋₆ alkyl group, or together with the carbon to which they are attached join to form a C₃₋₈ cycloalkyl or a heterocyclyl group;
S is the sequence -Glu-Nle-;
T is the sequence -Phe-Ile-;
W is the sequence -Trp-Leu-Ile-;
Z is absent or is -Pro-;
AA¹ is —NHCHR³CO—; wherein R³ is selected from —(CH₂)_{y}CONH₂, —(CH₂)_{y}COOH or —(CH₂)_{y}tetrazolyl; where y is 1 or 2;
AA² is -Gly-, -DAla-, -Lys- optionally joined to AA⁵ via a lactam bridge or -Glu- optionally joined to AA⁵ via a lactam bridge;
AA³ is -Ser-Phe- or -Ser-2-F-α-Me-Phe-;
AA⁴ is -Ser- or -Glu- optionally joined to AA⁶ via a lactam bridge;
AA⁵ is -Asp- optionally joined to AA² via a lactam bridge or -Lys- optionally joined to AA² or AA⁷ via a lactam bridge;
AA⁶ is -D-Phe-, -D-α-Me-Phe- or -Lys- optionally joined to AA¹⁰ via a lactam bridge;
AA⁷ is -Asp- optionally joined to AA⁵ via a lactam bridge, -Glu- optionally joined to AA¹⁰ via a lactam bridge or -Lys- optionally joined to AA¹⁰ via a lactam bridge;
AA⁸ is -Ile or -α-Me-Leu-;
AA⁹ is -Leu-Asp- or -Leu-ACPC-;
AA¹⁰ is -Asp- optionally joined to AA⁷ or AA¹⁴ via a lactam bridge, -Glu- optionally joined to AA⁷ or AA¹⁴ via a lactam bridge or -Lys- optionally joined to AA⁷ via a lactam bridge;
AA¹¹ is -LysR- where LysR is an N-substituted Lysine residue, -Glu- optionally joined to AA¹⁴ via a lactam bridge or -Lys- optionally joined to AA¹⁵ via a lactam bridge;
AA¹² is -Ala- or -AlB-;
AA¹³ is -Ala- or -AlB-;
AA¹⁴ is -AlB- or is -Lys- optionally joined to AA¹⁰ or AA¹¹ via a lactam bridge;
AA¹⁵ is -Asp- optionally joined to AA¹¹ via a lactam bridge or -Glu- optionally joined to AA¹⁶ via a lactam bridge;
AA¹⁶ is -Asn-, -ACPC-, -Lys- optionally joined to AA¹⁷ via a lactam bridge or -Glu- optionally joined to AA¹⁷ via a lactam bridge;
AA¹⁷ is -Gln-, -ACPC-, -Lys- optionally joined to AA¹⁶ via a lactam bridge or -Glu- optionally joined to AA¹⁶ via a lactam bridge;
AA¹⁸ is -Thr-, -Lys- optionally joined to AA²² via a lactam bridge or -Glu- optionally joined to AA²² via a lactam bridge;
AA¹⁹ is -Pro-, -PIPALA-, -Lys- or -Glu- optionally joined to AA²² via a lactam bridge;
AA²⁰ is absent or is -Ile-, -α-Me-Leu- or -Pro-;
AA²¹ is absent or is -Thr-;
AA²² is absent or is -Lys- optionally joined to AA¹⁸ or AA¹⁹ via a lactam bridge or -Glu- optionally joined to AA¹⁸ via a lactam bridge;
S^{a} is the sequence -Ser-Phe-;
T^{a} is the sequence -Glu-Nle-;
W^{a} is the sequence -Ala-Ala-;
X^{a} is the sequence -Asp-Phe-Ile-;
Y^{a} is the sequence -Trp-Leu-Ile-;
Z^{a} is absent or is the sequence -Ile-Thr-;
AA^{1a} is —NHCHR³CO—; wherein R³ is selected from —(CH₂)_{y}CONH₂, —(CH₂)_{y}COOH or —(CH₂)_{y} tetrazolyl; where y is 1 or 2;
AA^{2a} is -Gly-, -DAla-, -Lys- optionally joined to AA^{4a} via a lactam bridge or -Glu- optionally joined to AA^{4a} via a lactam bridge;
AA^{3a} is -Ser- or is -Glu- optionally joined to AA^{5a} via a lactam bridge;
AA^{4a} is -Asp- optionally joined to AA^{2a} via a lactam bridge, or -Lys- optionally joined to AA^{2a} or AA^{6a} via a lactam bridge;
AA^{5a} is -DPhe-, -Asp- optionally joined to AA^{8a} via a lactam bridge or -Lys- optionally joined to AA^{3a} via a lactam bridge;
AA^{6a} is -Thr-, -Asp- optionally joined to AA^{4a} or AA^{9a} via a lactam bridge, -Glu- optionally joined to AA^{9a} via a lactam bridge or -Lys- optionally joined to AA^{9a} via a lactam bridge;
AA^{7a} is -Ile- or an α-methyl Leucine residue of formula: 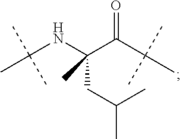
AA^{8a} is -Asp- or is -Lys- optionally joined to AA^{5a} via a lactam bridge;
AA^{9a} is -Leu-, -Lys- optionally joined to AA^{6a} or AA^{11a} via a lactam bridge, -Asp- optionally joined to AA^{6a} or AA^{11a} via a lactam bridge or -Glu- optionally joined to AA^{11a} via a lactam bridge;
AA^{10a} is -Lys- or is -Glu- optionally joined to AA^{11a} via a lactam bridge;
AA^{11a} is -Aib-, -Lys- optionally joined to AA^{9a} or AA^{10a} via a lactam bridge, -Glu- optionally joined to AA^{9a} via a lactam bridge or -Asp- optionally joined to AA^{9a} via a lactam bridge;
AA^{12a} is -Asn-, -Glu- optionally joined to AA^{13a} via a lactam bridge or -Lys- optionally joined to AA^{13a} via a lactam bridge;
AA^{13a} is -Gln-, -Asp- optionally joined to AA^{12a} via a lactam bridge or -Lys- optionally joined to AA^{12a} via a lactam bridge;
AA^{14a} is -Thr- or is -Lys- optionally joined to AA^{16a} via a lactam bridge;
AA^{15a} is -Lys- optionally joined to AA^{16a} via a lactam bridge or -Glu- optionally joined to AA^{16a} via a lactam bridge;
AA^{16a} is absent or is -Asp-, -Phe-, -Lys- optionally joined to AA^{15a} via a lactam bridge or -Glu- optionally joined to AA^{14a} or AA^{15a} via a lactam bridge;
wherein the C-terminus is a carboxyl group or a carboxamide group, or is adjoined to any natural or non-natural amino acid sequence or any other moiety, functional group or groups, and wherein the compound contains one, two, three, four or five lactam bridges;
or a tautomeric or stereochemically isomeric form thereof or a prodrug, salt or zwitterion thereof.

2. The compound according to claim 1 according to formula (la): 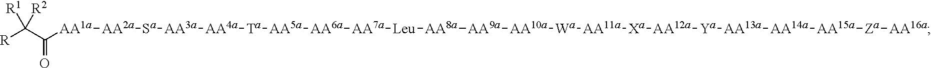
wherein;
R is selected from: 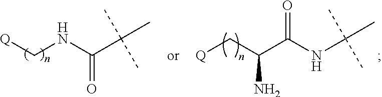
Q is phenyl or a monocyclic heteroaryl ring each of which may be optionally substituted with one or more R^{q} groups;
R^{q} is selected from halogen, hydroxyl, amino or C₁₋₆ alkyl having an alkyl chain optionally containing one or more heteroatoms selected from O, N, or S;
n is 1 to 3;
R¹ and R² are independently selected from hydrogen or a C₁₋₆ alkyl group, or together with the carbon to which they are attached join to form a C₃₋₈ cycloalkyl or a heterocyclyl group;
S^{a} is the sequence -Ser-Phe-;
T^{a} is the sequence -Glu-Nle-;
W^{a} is the sequence -Ala-Ala-;
X^{a} is the sequence -Asp-Phe-Ile-;
Y^{a} is the sequence -Trp-Leu-Ile-;
Z^{a} is absent or is the sequence -Ile-Thr-;
AA^{1a} is —NHCHR³CO—; wherein R³ is selected from —(CH₂)_{y}CONH₂, —(CH₂)_{y}COOH or —(CH₂)_{y}tetrazolyl; where y is 1 or 2;
AA^{2a} is -Gly-, -DAla-, -Lys- optionally joined to AA^{4a} via a lactam bridge or -Glu- optionally joined to AA^{4a} via a lactam bridge;
AA^{3a} is -Ser- or is -Glu- optionally joined to AA^{5a} via a lactam bridge;
AA^{4a} is -Asp- optionally joined to AA^{2a} via a lactam bridge, or -Lys- optionally joined to AA^{2a} or AA^{6a} via a lactam bridge;
AA^{5a} is -DPhe-, -Asp- optionally joined to AA^{8a} via a lactam bridge or -Lys- optionally joined to AA^{3a} via a lactam bridge;
AA^{6a} is -Thr-, -Asp- optionally joined to AA^{4a} or AA^{9a} via a lactam bridge, -Glu- optionally joined to AA^{9a} via a lactam bridge or -Lys- optionally joined to AA^{9a} via a lactam bridge;
AA^{7a} is -Ile- or an α-methyl Leucine residue of formula: 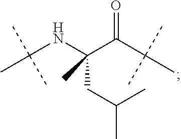
AA^{8a} is -Asp- or is -Lys- optionally joined to AA^{5a} via a lactam bridge;
AA^{9a} is -Leu-, -Lys- optionally joined to AA^{6a} or AA^{11a} via a lactam bridge, -Asp- optionally joined to AA^{6a} or AA^{11a} via a lactam bridge or -Glu- optionally joined to AA^{11a} via a lactam bridge;
AA^{10a} is -Lys- or is -Glu- optionally joined to AA^{11a} via a lactam bridge;
AA^{11a} is -Aib-, -Lys- optionally joined to AA^{9a} or AA^{10a} via a lactam bridge, -Glu- optionally joined to AA^{9a} via a lactam bridge or -Asp- optionally joined to AA^{9a} via a lactam bridge;
AA^{12a} is -Asn-, -Glu- optionally joined to AA^{13a} via a lactam bridge or -Lys- optionally joined to AA^{13a} via a lactam bridge;
AA^{13a} is -Gln-, -Asp- optionally joined to AA^{12a} via a lactam bridge or -Lys- optionally joined to AA^{12a} via a lactam bridge;
AA^{14a} is -Thr- or is -Lys- optionally joined to AA^{16a} via a lactam bridge;
AA^{15a} is -Lys- optionally joined to AA^{16a} via a lactam bridge or -Glu- optionally joined to AA^{16a} via a lactam bridge;
AA^{16a} is absent or is -Asp-, -Phe-, -Lys- optionally joined to AA^{15a} via a lactam bridge or -Glu- optionally joined to AA^{14a} or AA^{15a} via a lactam bridge;
wherein the AA^{15a} or AA^{16a} C-terminus is a carboxyl group or a carboxamide group, or is adjoined to any natural or non-natural amino acid sequence or any other moiety, functional group or groups, and wherein the compound contains one or two lactam bridges;
or a tautomeric or stereochemically isomeric form thereof or a prodrug, salt or zwitterion thereof.

3. The compound according to claim 1 of formula (1b) 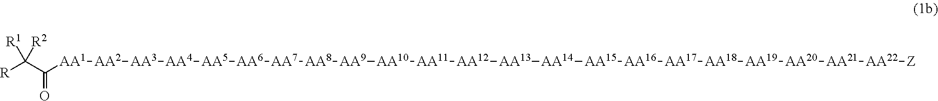
wherein;
R is selected from: 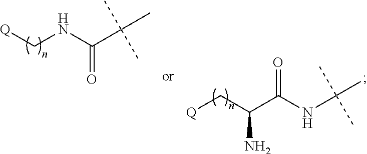
Q is phenyl or a monocyclic heteroaryl ring each of which may be optionally substituted with one or more R^{q} groups;
R^{q} is selected from halogen, hydroxyl, amino or C₁₋₆ alkyl having an alkyl chain optionally containing one or more heteroatoms selected from O, N, or S;
n is 1 to 3;
R¹ and R² are independently selected from hydrogen or a C₁₋₆ alkyl group, or together with the carbon to which they are attached join to form a C₃₋₈ cycloalkyl or a heterocyclyl group;
S is the sequence -Glu-Nle-;
T is the sequence -Phe-Ile-;
W is the sequence -Trp-Leu-Ile-;
Z is absent or is -Pro-;
AA¹ is —NHCHR³CO—; wherein R³ is selected from —(CH₂)_{y}CONH₂, —(CH₂)_{y}COOH or —(CH₂)_{y}tetrazolyl; where y is 1 or 2;
AA² is -Gly-, -DAla-, -Lys- optionally joined to AA⁵ via a lactam bridge or -Glu- optionally joined to AA⁵ via a lactam bridge;
AA³ is -Ser-Phe or -Ser-2-F-α-Me-Phe-;
AA⁴ is -Ser- or -Glu- optionally joined to AA⁶ via a lactam bridge;
AA⁵ is -Asp- optionally joined to AA² via a lactam bridge or -Lys- optionally joined to AA² or AA⁷ via a lactam bridge;
AA⁶ is -D-Phe-, -D-α-Me-Phe- or -Lys- optionally joined to AA¹⁰ via a lactam bridge;
AA⁷ is -Asp- optionally joined to AA⁵ via a lactam bridge, -Glu- optionally joined to
AA¹⁰ via a lactam bridge or -Lys- optionally joined to AA¹⁰ via a lactam bridge;
AA⁸ is -Ile or -α-Me-Leu-;
AA⁹ is -Leu-Asp- or -Leu-ACPC-;
AA¹⁰ is -Asp- optionally joined to AA⁷ or AA¹⁴ via a lactam bridge, -Glu- optionally joined to AA⁷ or AA¹⁴ via a lactam bridge or -Lys- optionally joined to AA⁷ via a lactam bridge;
AA¹¹ is -LysR- where LysR is an N-substituted Lysine residue, -Glu- optionally joined to AA¹⁴ via a lactam bridge or -Lys- optionally joined to AA¹⁵ via a lactam bridge;
AA¹² is -Ala- or -AlB-;
AA¹³ is -Ala- or -AlB-;
AA¹⁴ is -AlB- or is -Lys- optionally joined to AA¹⁰ or AA¹¹ via a lactam bridge;
AA¹⁵ is -Asp- optionally joined to AA¹¹ via a lactam bridge or -Glu- optionally joined to AA¹⁶ via a lactam bridge;
AA¹⁶ is -Asn-, -ACPC-, -Lys- optionally joined to AA¹⁷ via a lactam bridge or -Glu- optionally joined to AA¹⁷ via a lactam bridge;
AA¹⁷ is -Gln-, -ACPC-, -Lys- optionally joined to AA¹⁶ via a lactam bridge or -Glu- optionally joined to AA¹⁶ via a lactam bridge;
AA¹⁸ is -Thr-, -Lys- optionally joined to AA²² via a lactam bridge or -Glu- optionally joined to AA²² via a lactam bridge;
AA¹⁹ is -Pro-, -PIPALA-, -Lys- or -Glu- optionally joined to AA²² via a lactam bridge;
AA²⁰ is absent or is -Ile-, -α-Me-Leu- or -Pro-;
AA²¹ is absent or is -Thr-;
AA²² is absent or is -Lys- optionally joined to AA¹⁸ or AA¹⁹ via a lactam bridge or -Glu- optionally joined to AA¹⁸ via a lactam bridge;
wherein the C-terminus is a carboxyl group or a carboxamide group, or is adjoined to any natural or non-natural amino acid sequence or any other moiety, functional group or groups, and wherein the compound contains three, four or five lactam bridges;
or a tautomeric or stereochemically isomeric form thereof or a prodrug, salt or zwitterion thereof.

4. The compound according to claim 1, wherein Q is:

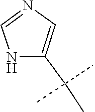

5. The compound according to claim 1, wherein n is 1 or 2.

6. The compound as defined in claim 1, wherein R¹ and R² are independently selected from hydrogen or a C₁₋₆ alkyl group.

7. The compound according to claim 6, wherein R¹ and R² are both methyl.

8. The compound according to claim 1, wherein R³ represents —CH₂tetrazolyl or CH₂COOH.

9. The compound according to claim 1, wherein AA² is cyclised to AA⁵, and either AA¹⁰ is cyclised to one of AA⁶, AA⁷ or AA¹⁴ or AA¹¹ is cyclised to one of AA¹⁴ or AA¹⁵ and either AA¹⁶ is cyclised to AA¹⁷ or AA²² is cyclised to either AA¹⁸ or AA¹⁹.

10. The compound according to claim 1, wherein AA¹⁶ is cyclised to AA¹⁷ and AA²² is cyclised to either AA¹⁸ or AA¹⁹.

11. The compound according to claim 1, wherein AA^{2a} is -Gly- or -DAla-, AA^{3a} is -Ser-, AA^{4a} is -Asp-, AA^{5a} is -DPhe-, AA^{6a} is -Thr-, AA^{8a} is -Asp-, AA^{10a} is -Lys- and AA^{15a} is -Lys-.

12. The compound according to claim 1, wherein AA^{9a} is -Leu-, -Glu- joined to AA^{11a} via a lactam bridge or -Lys- joined to AA^{11a} via a lactam bridge.

13. The compound according to claim 1, wherein AA^{11a} is -Lys- optionally joined to AA^{9a} via a lactam bridge or -Glu- joined to AA^{9a} via a lactam bridge.

14. The compound according to claim 1, wherein AA^{12a} is -Asn- or is -Glu- joined to AA^{13a} via a lactam bridge.

15. The compound according to claim 1, wherein AA^{13a} is -Gln- or is -Lys- joined to AA^{12a} via a lactam bridge.

16. The compound according to claim 1, wherein AA^{14a} is -Thr- or is -Lys- joined to AA^{16a} via a lactam bridge.

17. The compound according to claim 1, wherein AA^{16a} is -Phe- or is -Glu- joined to AA^{14a} via a lactam bridge.

18. The compound according to claim 1, wherein Z^{a} and AA^{16a} are absent.

19. The compound according to claim 1, wherein the C-terminus is a carboxamide group.

20. The compound according to claim 1 which is selected from any one of Examples 1 to 117.

21. The compound according to claim 1 which is selected from the group consisting of:
Example 30: 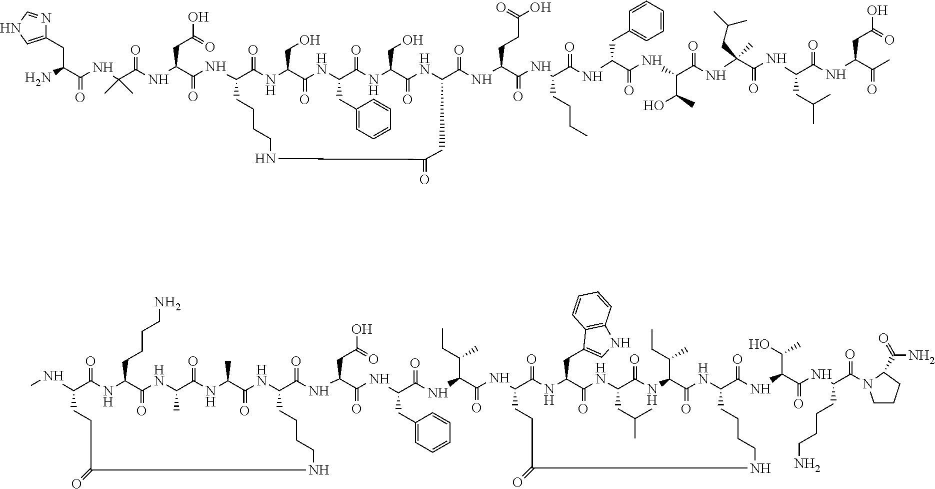
Example 31: 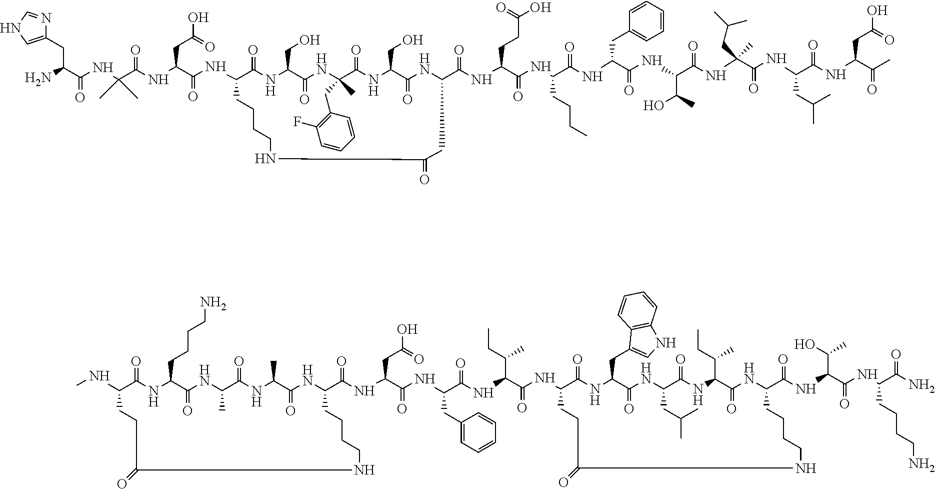
Example 46: 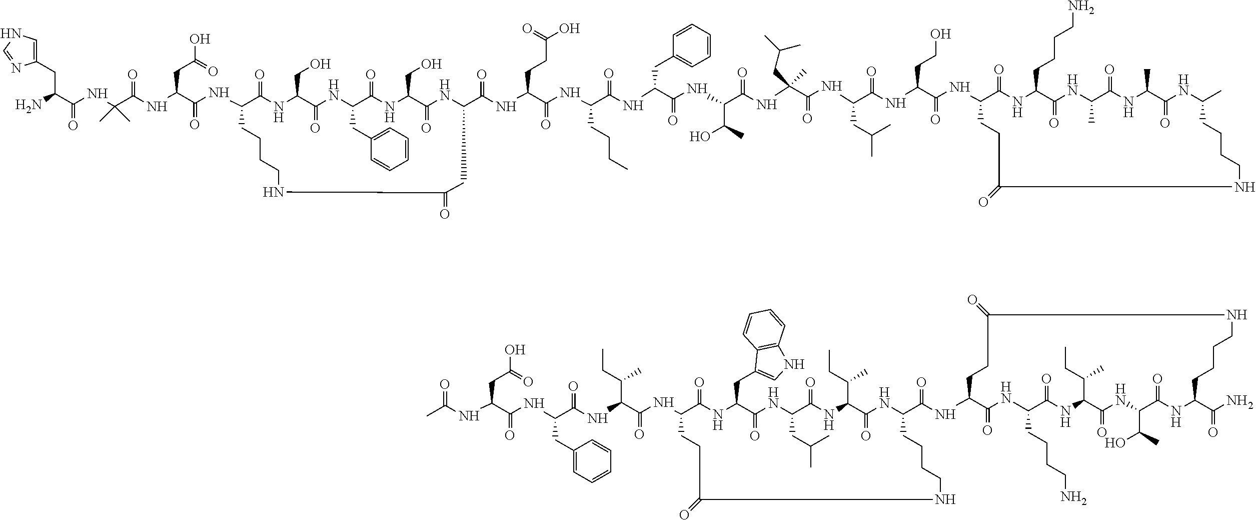
Example 48: 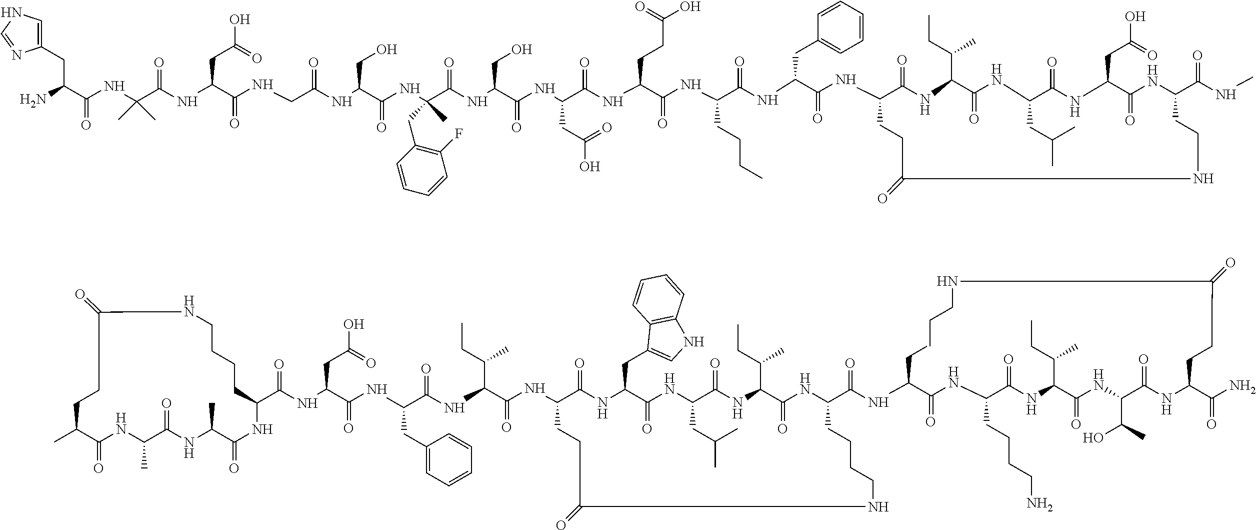
Example 52: 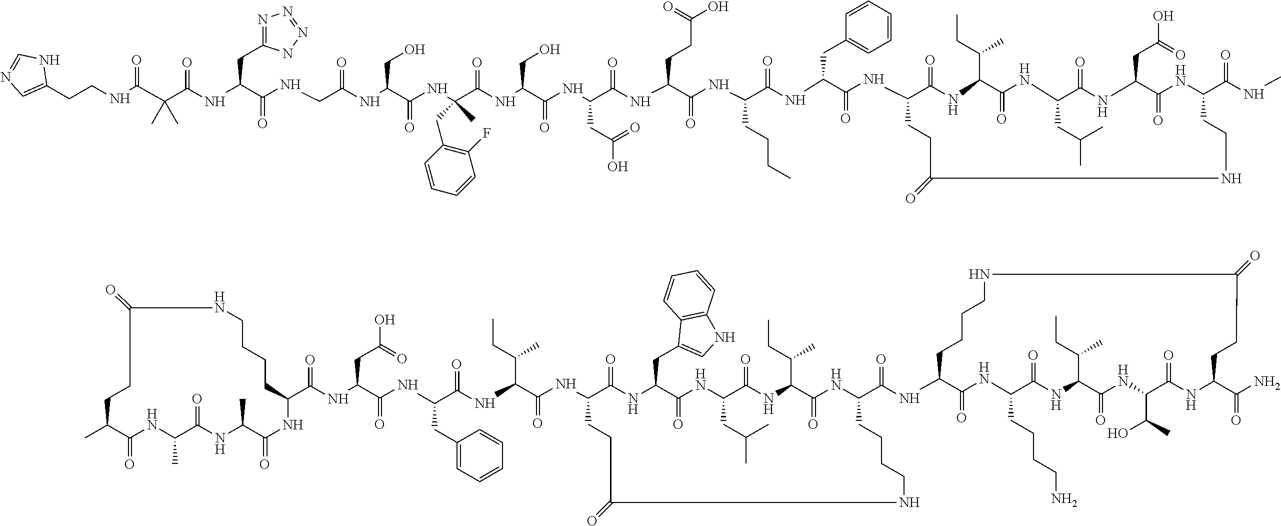
Example 55: 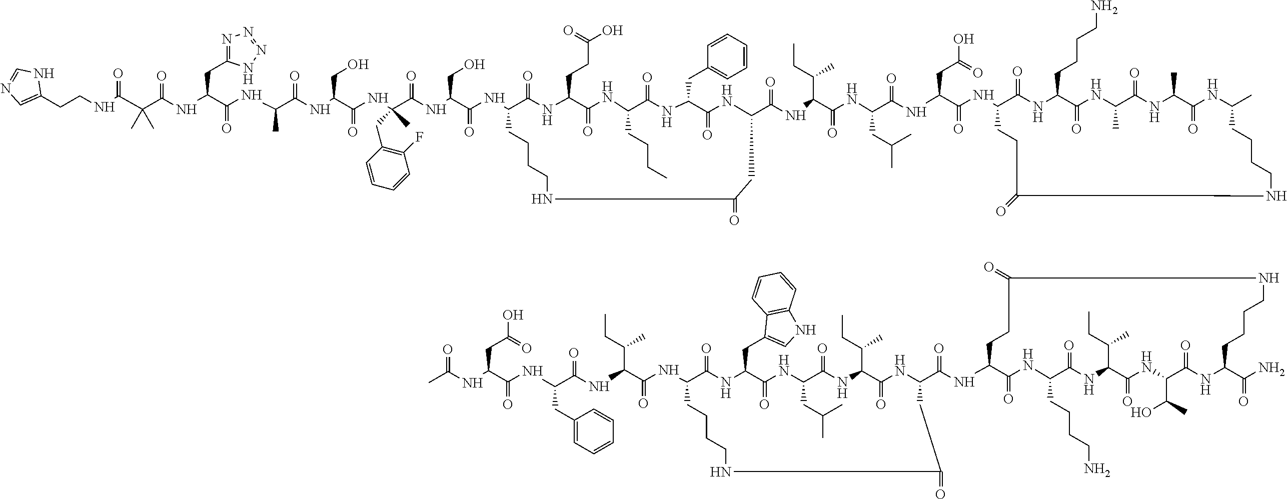
Example 60: 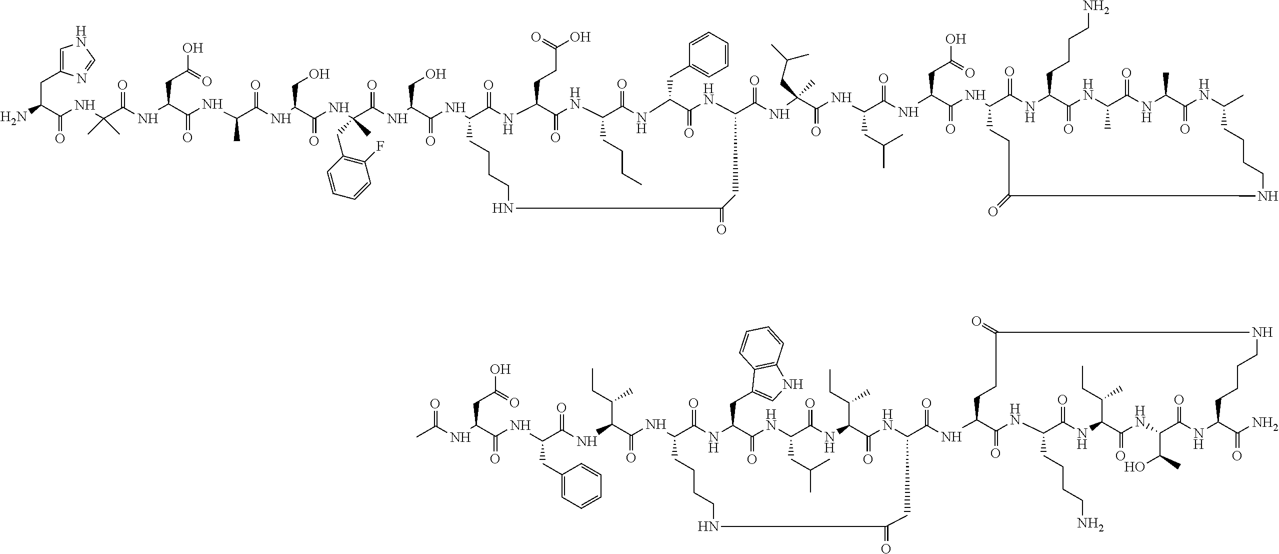
Example 74: 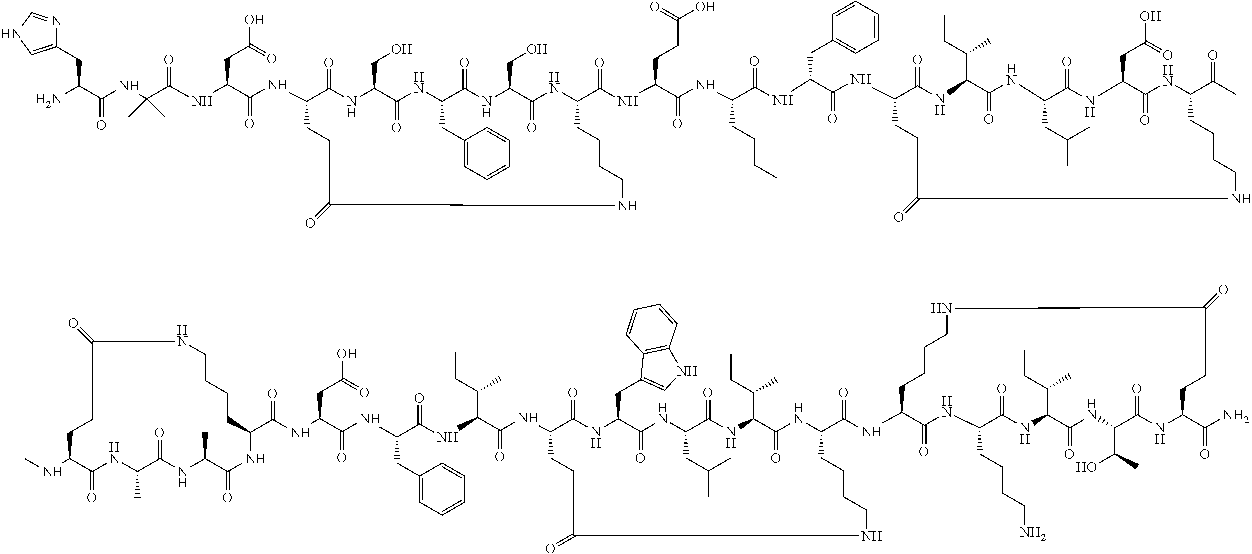
Example 76: 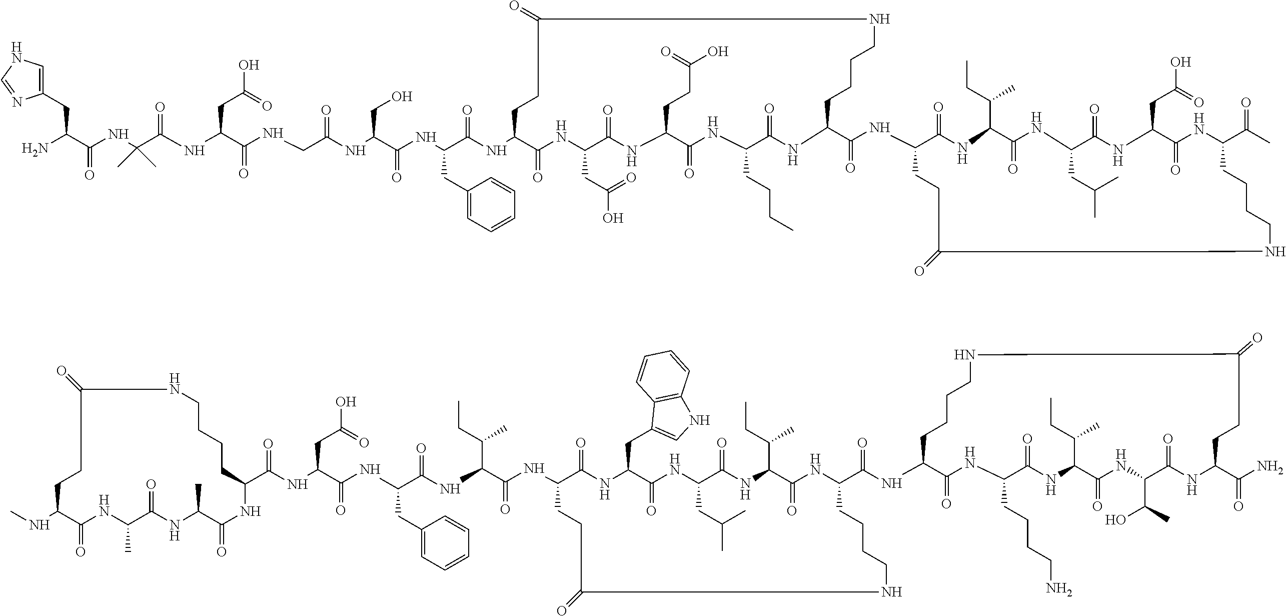
Example 93: 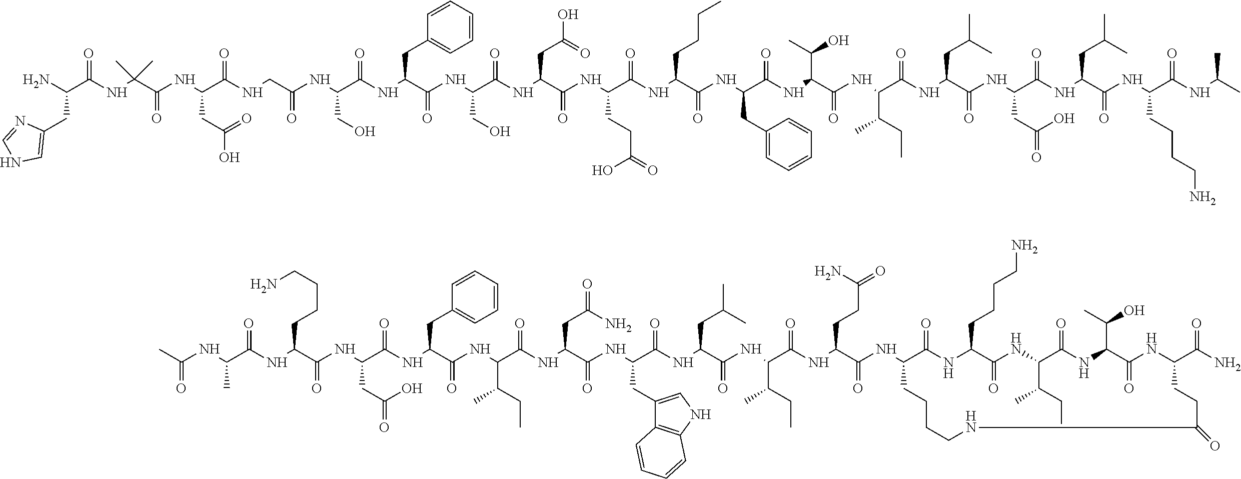
Example 96: 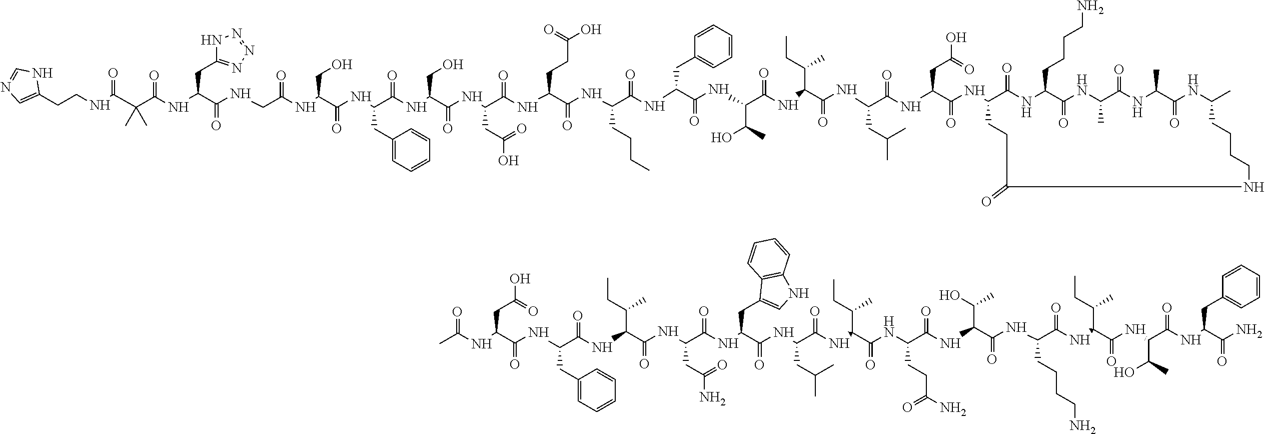
Example 115: 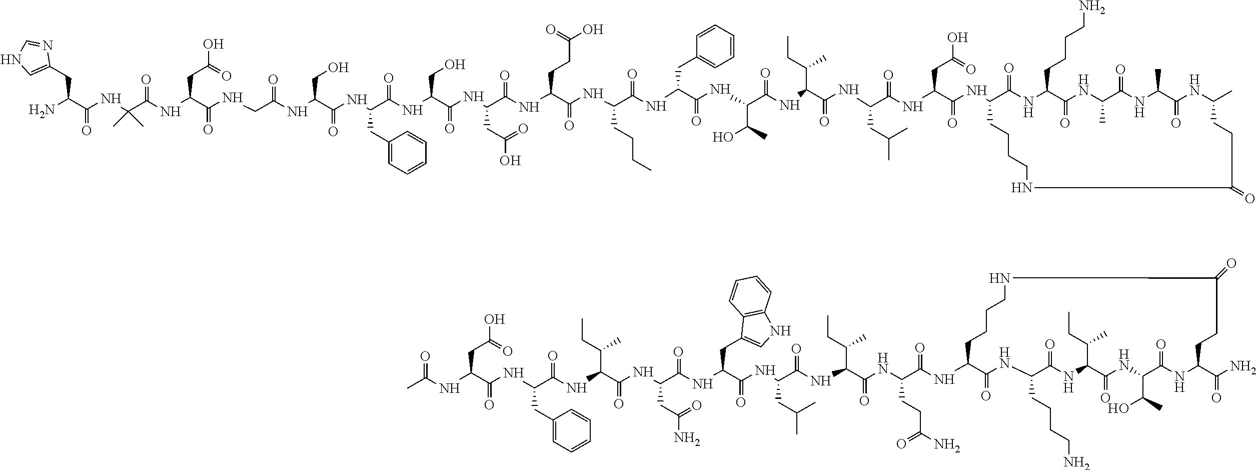
Example 117: 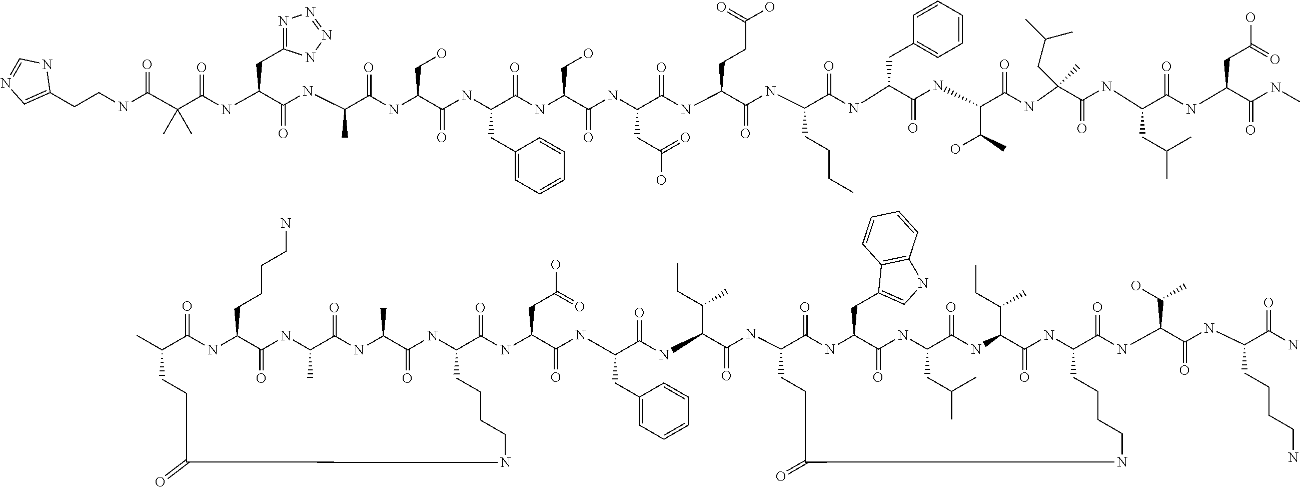
or a tautomer, salt or zwitterion thereof.

22. The compound according to claim 1 having GLP-1 and/or GLP-2 receptor agonist activity.

23. The compound according to claim 22 having higher GLP-2 receptor agonist activity compared to GLP-1 receptor agonist activity.

24. A pharmaceutical composition comprising a compound as defined in claim 1 and a pharmaceutically acceptable excipient.

25. The compound or composition according to claim 1 for use in the treatment of gastrointestinal and metabolic diseases, promoting intestinal recovery and nutritional status of patients with malabsorption disorders, intestinal failure, intestinal insufficiency, diarrheal diseases, chronic inflammatory bowel disorders, improve mucosal barrier function, ameliorate gut inflammation, inflammatory disorders, celiac disease, congenital and acquired digestion and malabsorption syndromes, chronic diarrhoeal diseases, conditions caused by mucosal damage (e.g. cancer treatment), hyperglycemia during enteral and parenteral nutrition therapy in patients with intestinal failure, insufficiency or malabsorption disorders, gastrointestinal injury, diarrheal diseases, intestinal insufficiency, intestinal failure, acid-induced intestinal injury, arginine deficiency, obesity, celiac disease, chemotherapy-induced enteritis, diabetes, obesity, fat malabsorption, steatorrhea, autoimmune diseases, food allergies, gastric ulcers, gastrointestinal barrier disorders, Parkinson's disease, sepsis, bacterial peritonitis, inflammatory bowel disease, chemotherapy-associated tissue damage, bowel trauma, bowel ischemia, mesenteric ischemia, short bowel syndrome, malnutrition, necrotizing enterocolitis, necrotizing pancreatitis, neonatal feeding intolerance, NSAID-induced gastrointestinal damage, nutritional insufficiency, total parenteral nutrition damage to gastrointestinal tract, neonatal nutritional insufficiency, radiation-induced enteritis, radiation-induced injury to the intestines, mucositis, pouchitis, ischemia, obesity, type 2 diabetes, non-alcoholic fatty liver disease (NAFLD), nonalcoholic steatohepatitis (NASH), insulin resistance, hyperglycemia, insulin resistance, glucose intolerance, brush border enzyme deficiencies (congenital lactase deficiency, congenital sucrase-isomaltase deficiency, congenital maltase-glucoamylase-deficiency), defects of membrane carriers (glucose-galactose-malabsorption, fructose malabsorption, Fanconi-Bickel syndrome, Acrodermatitis enteropathica, Congenital chloride / sodium diarrhoea, Lysinuric protein intolerance, Primary biliary malabsorption, cystic fibrosis), enzyme deficiencies (hereditary pancreatitis, congenital pancreas lipase deficiency), lipid/lipoprotein metabolism defects (chylomicron retention disease, hypobetalipoproteinemia, abetalipoproteinemia), defects of enterocyte differentiation or cellular polarisation (Microvillous atrophy, Tufting enteropathy, Trichohepatoenteric syndrome, Familiar haemophagocytic lymphohistiocytosis type 5), defects of enteroendocrine cells (Congenital malabsorptive diarrhoea, anendocrinosis, protein-convertase ⅓ deficiency), congenital diarrheal diseases.

26. The use according to claim 25, wherein the disorder is Tufting enteropathy.
